(19) United States
(12) Patent Application Publication
(10) Pub. No.: US 2023/0068726 A1
(43) Pub. Date: Mar. 2, 2023

(54) TRANSPOSON SYSTEMS FOR GENOME EDITING

(71) Applicant: The Regents of the University of California, Oakland, CA (US)

(72) Inventors: Jennifer A. Doudna, Berkeley, CA (US); Jillian F. Banfield, Berkeley, CA (US); Brady F. Cress, Berkeley, CA (US); Benjamin E. Rubin, Berkeley, CA (US); Spencer Diamond, Berkeley, CA (US); Adam M. Deutschbauer, Berkeley, CA (US)

(21) Appl. No.: 17/794,166

(22) PCT Filed: Jan. 28, 2021
(86) PCT No.: PCT/US21/15524

§ 371 Date: Jul. 20, 2022

Related U.S. Application Data
(60) Provisional application No. 62/968,644, filed on Jan. 31, 2020.
(60) Provisional application No. 63/052,839, filed on Jul. 16, 2020.

(51) Int. Cl. C12N 9/22; C12N 15/10; C12N 15/11; C12N 1/20; C12N 15/74

(52) U.S. Cl. CPC C12N 9/22; C12N 15/1065; C12N 15/11; C12N 15/111; C12N 1/20; C12N 15/74; C12N 2310/20; C12N 2800/80; C12N 2800/101

(57) ABSTRACT

The present disclosure provides a transposon system comprising: i) a nucleotide sequence encoding polypeptides that form a CRISPR-associated transposase complex; ii) a nucleotide sequence encoding a guide RNA; and iii) a transposon, or an insertion site for a transposon, flanked by CAST complex recognition sites. The present disclosure provides a prokaryotic cell comprising a subject transposon system. The transposon system is useful for editing the genome of a target prokaryotic cell. The present disclosure provides methods for editing the genome of a target prokaryotic cell. The present disclosure further provides systems and methods for identifying, within a heterogeneous population of prokaryotic cells, prokaryotic species that are susceptible to genetic modification and gene editing.

### CROSS-REFERENCE

This application claims the benefit of U.S. Provisional Patent Application No. 62/968,744, filed Jan. 30, 2020, and U.S. Provisional Patent Application No. 63/052,839, filed Jul. 16, 2020, which applications are incorporated herein by reference in their entirety.

### STATEMENT REGARDING FEDERALLY SPONSORED RESEARCH

This invention was made with government support under DE-AC02-05CH11231 awarded by the U.S. Department of Energy. The government has certain rights in the invention.

### INTRODUCTION

Site-specific insertion of large DNA segments into the genome of a single microorganism in isolation, or in a mixture of cells (a synthetic or natural microbial community, for example), remains challenging. This may be due to two factors. First, CRISPR-Cas-mediated genome editing in prokaryotes remains very low efficiency, because the vast majority of prokaryotic cells that experience CRISPR-Cas-mediated genomic double strand breaks (DSBs) experience cell death Small fractions of a targeted cell population are rescued, only if host DNA repair mechanisms are able to integrate a homologous repair template DNA (ssDNA or dsDNA) that lacks the CRISPR-Cas target site. Second, almost all prior target site-customizable genome editing techniques developed for prokaryotes suffer from decreased integration efficiency with increasing size of integrated DNA. CRISPR-Cas transposases—transposases that utilize nuclease inactive CRISPR-Cas systems for target site selection and binding—are the first genome editing systems that circumvent both of these limitations; they do not induce DSBs and thus do not rely on host DNA repair mechanisms for transposon integration, and they naturally transpose large DNA cargo (˜10-20 kb).

There is a need in the art for conjugative vector systems for delivering transposons to target prokaryotic cells.

### SUMMARY

The present disclosure provides a transposon system comprising: i) a nucleotide sequence encoding polypeptides that form a CRISPR-associated transposase complex; ii) a nucleotide sequence encoding a guide RNA; and iii) a transposon, or an insertion site for a transposon, flanked by CAST complex recognition sites. The present disclosure provides a prokaryotic cell comprising a subject transposon system. The transposon system is useful for editing the genome of a target prokaryotic cell. The present disclosure provides methods for editing the genome of a target prokaryotic cell. The present disclosure further provides systems and methods for identifying, within a heterogeneous population of prokaryotic cells, prokaryotic species that are susceptible to genetic modification and gene editing.

### BRIEF DESCRIPTION OF THE DRAWINGS

FIG. 1 depicts a map showing features of an “all-in-one” conjugative vector encoding an RNA-guided CRISPR-Cas transposase.

FIG. 2 is a schematic depiction of conjugative delivery and selection following RNA-guided CRISPR-Cas-mediated transposition.

FIG. 3 depicts transposition efficiency in recipient bacterial strain BL21(DE3) using a single conjugative vector of the present disclosure.

FIG. 4A-4D provide amino acid sequences of Scytonema hofmanni CAST polypeptides.

FIG. 5A-5G provide amino acid sequences of Vibrio cholerae CAST polypeptides.

FIG. 6A-6R provide amino acid sequences of CAST polypeptides suitable for use in an ShCAST-type complex.

FIG. 7A-7U provide amino acid sequences of CAST polypeptides suitable for use in a VcCAST-type complex.

FIG. 8A-8F provide details of pBFC0619, an example of a single conjugative transposon construct (from top to bottom SEQ ID NOs:58, 13-18, 59-61).

FIG. 9A-9F provide details of pBFC0687, an example of a single conjugative transposon construct (from top to bottom SEQ ID NOs:9-11, 8, 59, 61).

FIG. 10A-10B provide maps of pBFC0619 and pBFC0687.

FIG. 11-19 provide illustrations of targeted genome editing within microbial communities. FIG. 16 depicts “Environmental Transformation Sequencing” (“ET-Seq”) analysis on a 10-member “community” (heterogeneous population of prokaryotic cells). FIG. 17 depicts ET-seq analysis of a prokaryotic cell community in thiocyanate (SCN) bioreactor.

FIG. 20-22 provide workflows for targeted genome editing.

FIG. 23-25 depict the use of multi-spacer CRISPR arrays and pooled spacer libraries. FIG. 24 depicts use of a multi-spacer array (conjugative vector encoding multiple guide RNAs that target different target nucleic acids) to perform functional knockouts, generating auxotrophs. FIG. 25 depicts use of a pool (a library) of conjugative vectors, each encoding a different guide RNA that targets a different target nucleic acid, to perform functional knockouts, generating auxotrophs.

FIG. 26A-26D depict the use of ET-Seq for quantitative measurement of non-targeted editing in a community.

FIG. 27A-27B depict library preparation and data normalization for ET-Seq.

FIG. 28A-28C depict ET-Seq with multiple delivery approaches.

FIG. 29A-29B depict ET-Seq with multiple delivery approaches on thiocyanate bioreactor.

FIG. 30A-30D depict benchmarking all-in-one conjugal targeted vectors

FIG. 31A-31F depict benchmarking all-in-one conjugal CasTn vectors.

FIG. 32A-32B depict targeted editing in a 9-member consortium.

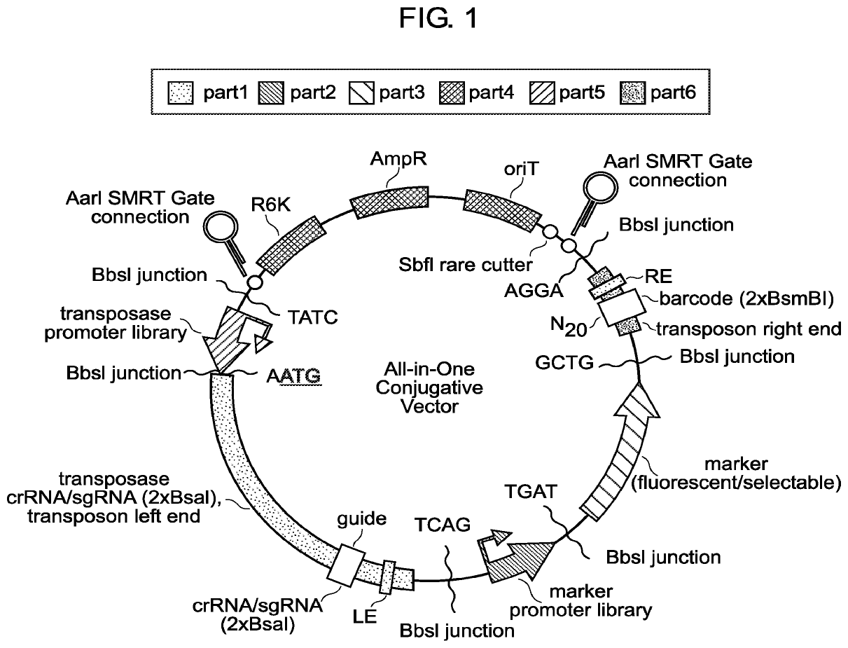

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a CAST polypeptide/guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{D}.

As used herein, a “promoter” or a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive expression by the various vectors of the present disclosure.

“Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a conjugative nucleic acid construct” includes a plurality of such constructs and reference to “the CAST complex” includes reference to one or more CAST complexes and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a transposon system comprising: i) a nucleotide sequence encoding polypeptides that form a CRISPR-associated transposase (CAST) complex; ii) a nucleotide sequence encoding a guide RNA; and iii) a transposon, or an insertion site for a transposon, flanked by CAST complex recognition sites. The present disclosure provides a prokaryotic cell comprising a subject transposon system. The transposon system is useful for editing the genome of a target prokaryotic cell. The present disclosure provides methods for editing the genome of a target prokaryotic cell.

### Transposon System

The present disclosure provides a transposon system comprising: i) a nucleotide sequence encoding polypeptides that form a CAST complex; ii) a nucleotide sequence(s) encoding one or more guide RNAs; and iii) a transposon, or an insertion site for a transposon, flanked by CAST complex recognition sites.

In some cases, i) a nucleotide sequence encoding polypeptides that form a CAST complex; ii) a nucleotide sequence(s) encoding one or more guide RNAs; and iii) a transposon, or an insertion site for a transposon, flanked by CAST complex recognition sites are all present on the same nucleic acid construct; i.e., are present on a single nucleic acid construct. In some cases, the nucleic acid construct is a conjugative construct. A conjugative construct comprises an origin of transfer, e.g., a nucleotide sequence that provides for transfer of the construct from a first prokaryotic cell to a second prokaryotic cell. In some cases, a conjugative construct of the present disclosure is a non-replicative construct. Thus, the present disclosure provides a single conjugative construct comprising: i) a nucleotide sequence encoding polypeptides that form a CAST complex; ii) a nucleotide sequence(s) encoding one or more guide RNAs; and iii) a transposon, or an insertion site for a transposon, flanked by CAST complex recognition sites. In some cases, a conjugative construct of the present disclosure is a replicative construct. In some cases, a conjugative construct of the present disclosure is replicative, but is lost from a host cell comprising the conjugative construct when the host cell is cultured at 37° C. or at a temperature that is higher than 37° C.

In some cases, i) a nucleotide sequence encoding polypeptides that form a CAST complex; ii) a nucleotide sequence(s) encoding one or more guide RNAs; and iii) a transposon, or an insertion site for a transposon, flanked by CAST complex recognition sites are all present on the same nucleic acid construct; i.e., are present on a single nucleic acid construct. In some cases, the nucleic acid construct is a conjugative construct. A conjugative construct comprises an origin of transfer, e.g., a nucleotide sequence that provides for transfer of the construct from a first bacterium to a second bacterium. In general, a conjugative construct is a non-replicative construct. Thus, the present disclosure provides a single conjugative construct comprising: i) a nucleotide sequence encoding polypeptides that form a CAST complex; ii) a nucleotide sequence encoding a guide RNA; and iii) a transposon, or an insertion site for a transposon, flanked by CAST complex recognition sites.

In some cases, i) a nucleotide sequence encoding polypeptides that form a CAST complex; and ii) a nucleotide sequence(s) encoding one or more guide RNAs are present on a first nucleic acid construct; and iii) a transposon, or an insertion site for a transposon, flanked by CAST complex recognition sites is present on a second nucleic acid construct. Thus, in some cases, a system of the present disclosure comprises: a) a first nucleic acid comprising: i) a nucleotide sequence encoding polypeptides that form a CAST complex; and ii) a nucleotide sequence(s) encoding one or more guide RNAs; and b) a second nucleic acid comprising a transposon, or an insertion site for a transposon, flanked by CAST complex recognition sites. In some cases, the nucleic acid constructs are both conjugative constructs.

In some cases, a nucleic acid construct of a transposon system of the present disclosure comprises a selectable marker. In some cases, a nucleic acid construct of a transposon system of the present disclosure does not comprise a selectable marker. Selectable markers include polypeptides that provide for antibiotic resistance. Antibiotic resistance includes, e.g., ampicillin resistance, kanamycin resistance, chloramphenicol resistance, streptomycin resistance, spectinomycin resistance, tetracycline resistance, erythromycin resistance, neomycin resistance, gentamycin resistance and the like. Polypeptides that provide for antibiotic resistance are known in the art and include, e.g., gentamycin acetyltransferase, beta-lactamase, neomycin phosphotransferase, and the like. Thus, a transposon system of the present disclosure can be used for negative selection (e.g., antimicrobial resistance).

In some cases, a nucleic acid construct of a transposon system of the present disclosure comprises a screenable marker (e.g., for positive selection), such as a fluorescent polypeptide. Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

As another example, in some cases, a nucleic acid construct of a transposon system of the present disclosure comprises a nucleotide sequence encoding a polypeptide that, when exhibited on the surface of a cell, can be targeted by an antibody specific for the polypeptide. Such polypeptides include, e.g., epitope tags.

In some cases, a nucleic acid construct of a transposon system of the present disclosure comprises a nucleic acid comprising nucleotide sequences encoding one or more polypeptides that can provide for metabolic selection (positive selection). For example, the ability to utilize a particular carbon source that is not normally a carbon source utilized by a particular bacterium can be selected. Such carbon sources include, e.g., lactose.

### CAST

CRISPR-associated transposases (CASTs) include a CRISPR-associated polypeptide and one or more additional polypeptides that, in complex with one another, mediate transposition of a target transposon.

### ShCAST

In some cases, a CAST comprises: i) a Cas 12k polypeptide; ii) a TnsC polypeptide; iii) a TnsB polypeptide; and iv) a TniQ polypeptide. An example of such a CAST is a Scytonema hofmanni CAST (ShCAST).

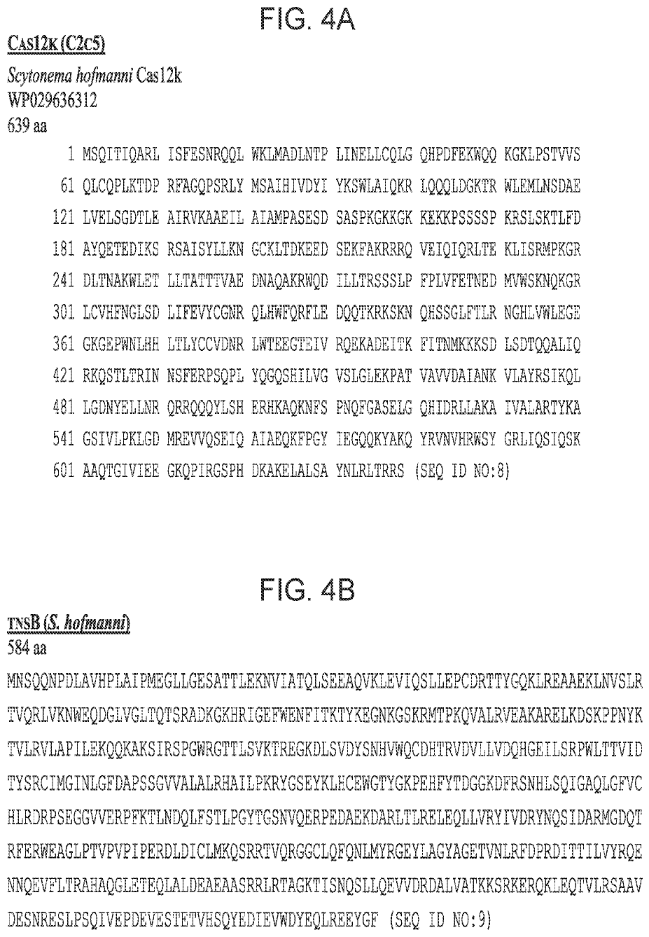

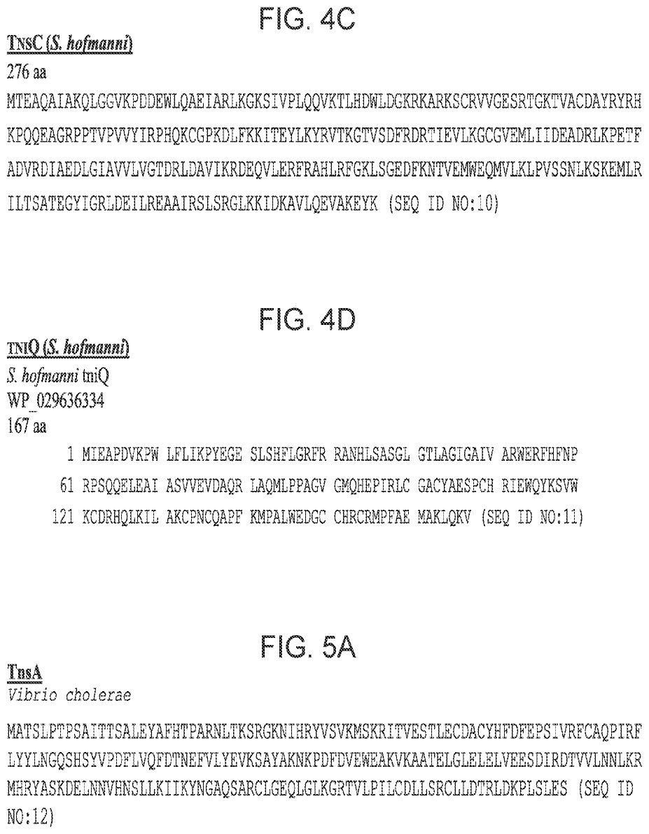

A Cas12k polypeptide can comprise an amino acid sequence having at least 50%, at least 60%, at least 70%, at least 80%, at least 90%, at least 95%, at least 98%, at least 99%, or 100% amino acid sequence identity to the S. hofmanni Cas12k amino acid sequence depicted in FIG. 4A. A Cas12k polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from 500 amino acids to 639 amino acids (e.g., from 500 amino acids (aa) to 550 aa, from 550 aa to 575 aa, from 575 aa to 600 aa, from 600 aa to 625 aa, or from 625 aa to 639 aa) of the S. hofmanni Cas12k amino acid sequence depicted in FIG. 4A. In some cases, the Cas12k polypeptide has a length of from about 600 amino acids to 650 amino acids (e.g., from 600 amino acids (aa) to 625 aa, or from 625 aa to 650 aa). In some cases, the Cas12k polypeptide has a length of 639 aa.

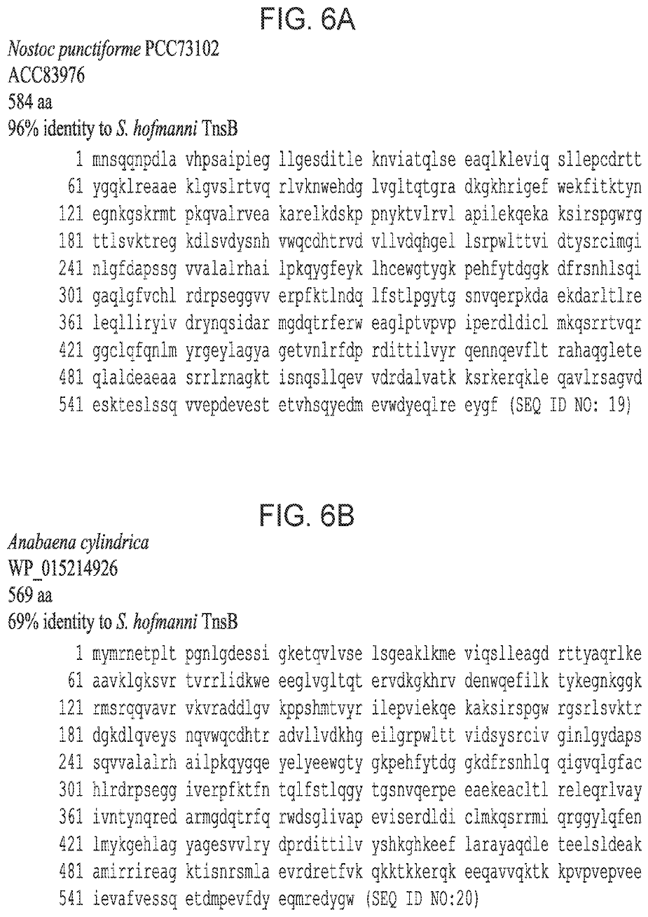

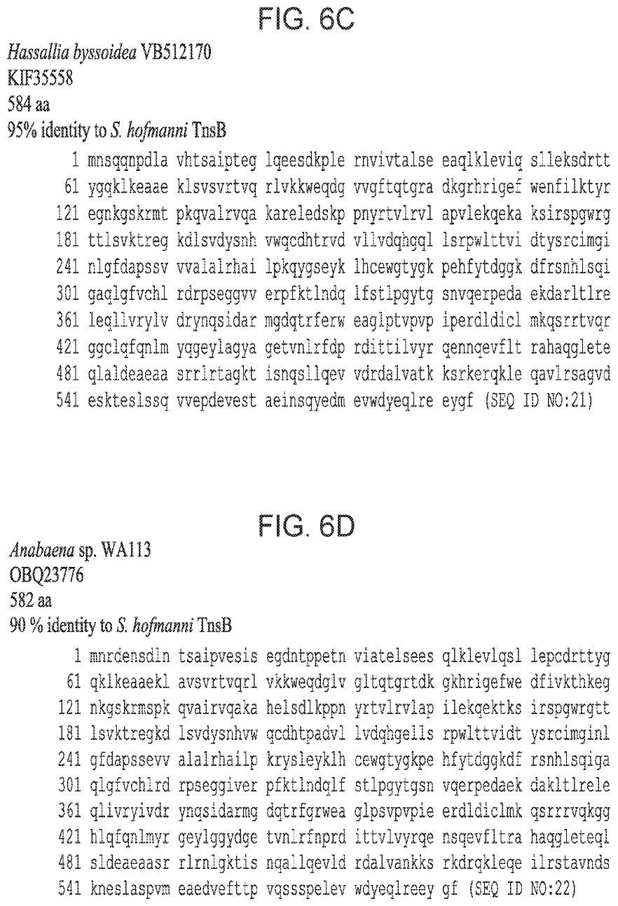

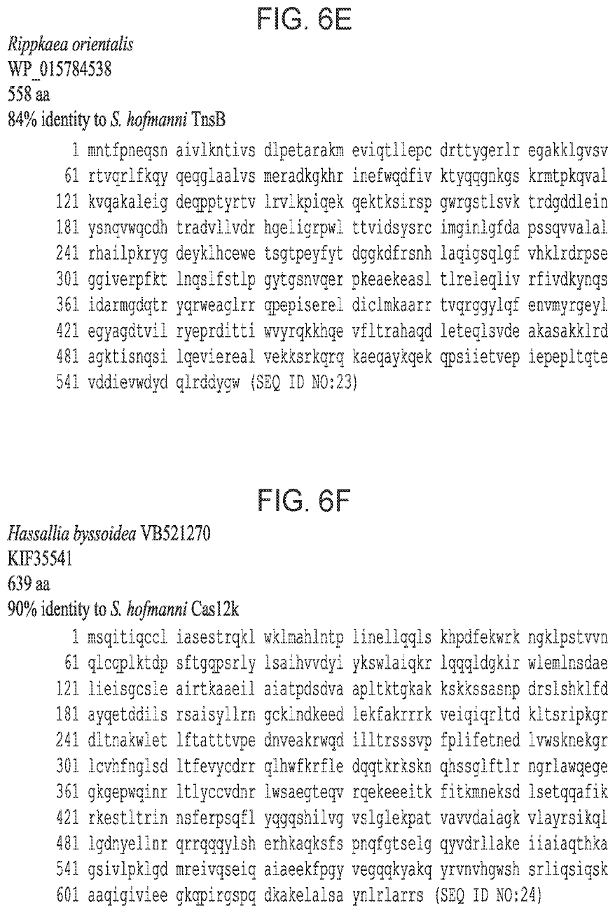

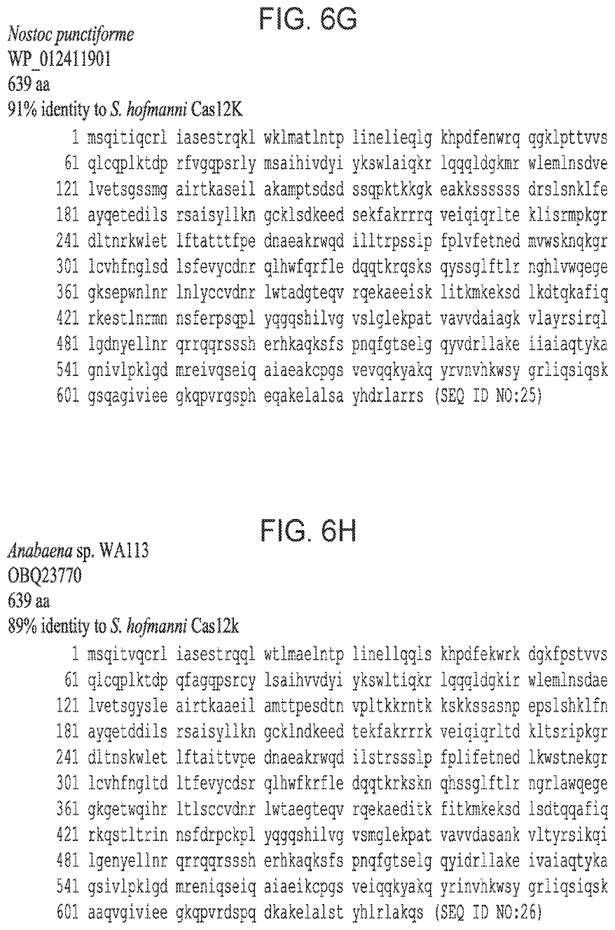

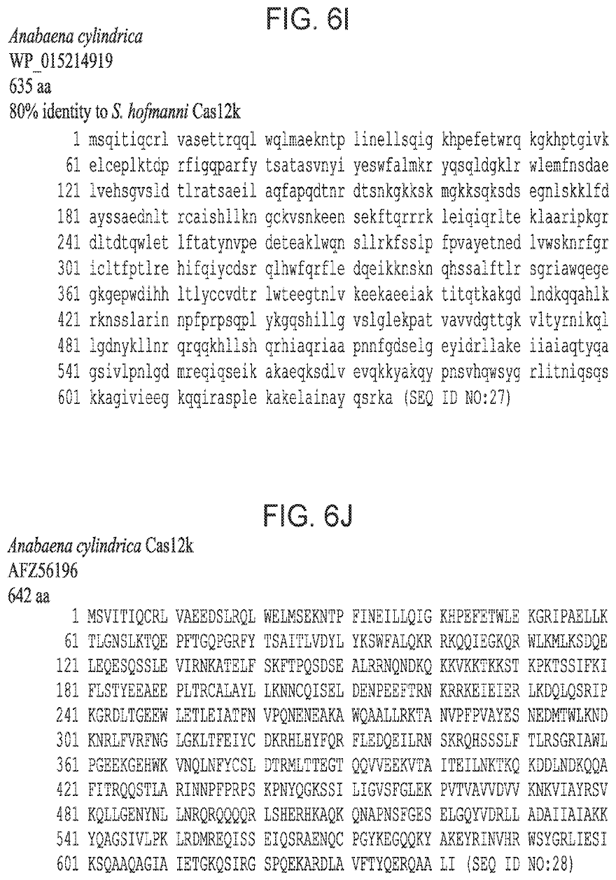

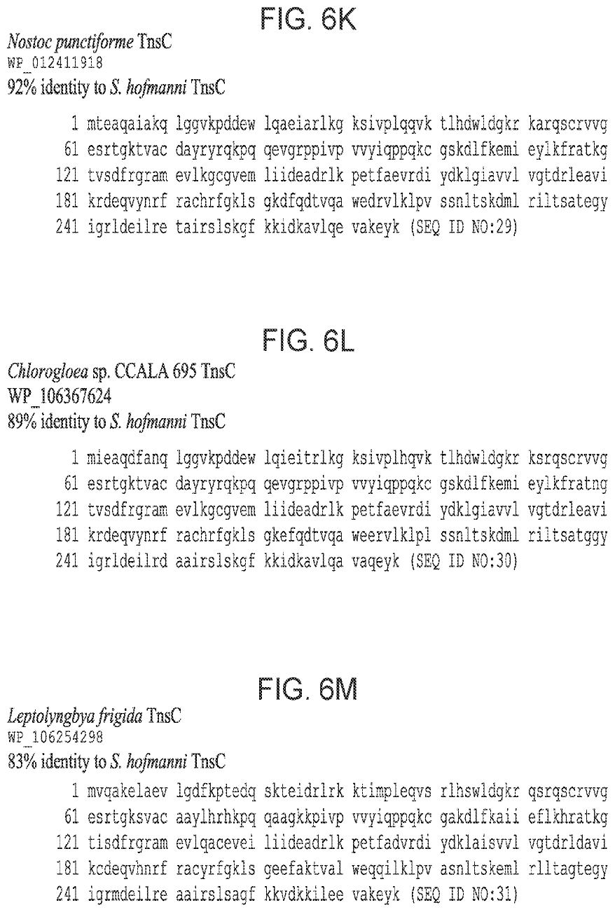

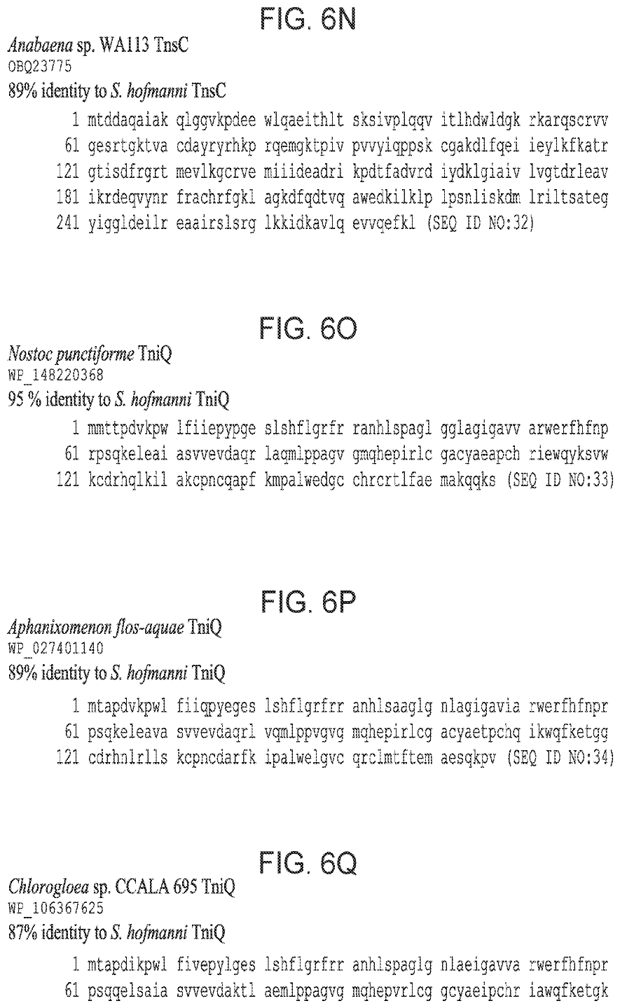

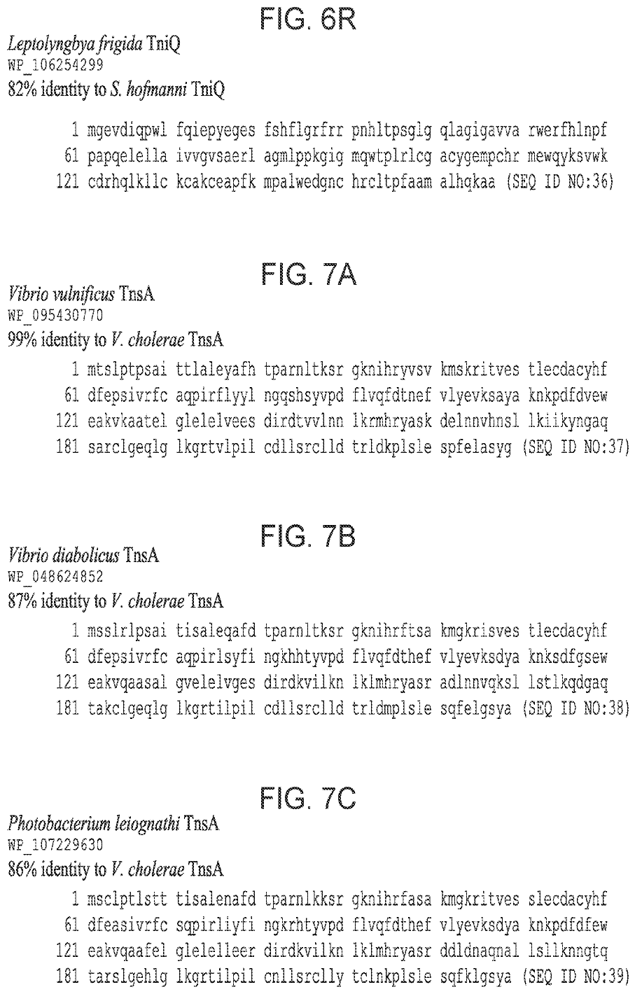

Non-limiting examples of other suitable Cas12k polypeptides are provided in FIG. 6F-6J. For example, a Cas12k polypeptide can comprise an amino acid sequence having at least 50%, at least 60%, at least 70%, at least 80%, at least 90%, at least 95%, at least 98%, at least 99%, or 100% amino acid sequence identity to any one of the Cas12k polypeptide amino acid sequences depicted in FIG. 6F-6J.

A TnsB polypeptide can comprise an amino acid sequence having at least 50%, at least 60%, at least 70%, at least 80%, at least 90%, at least 95%, at least 98%, at least 99%, or 100% amino acid sequence identity to the S. hofmanni TnsB amino acid sequence depicted in FIG. 4B. A TnsB polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 500 amino acids to 584 amino acids (e.g., from about 500 amino acids (aa) to 525 aa, from 525 aa to 550 aa, from 550 aa to 575 aa, or from 575 aa to 584 aa) of the S. hofmanni TnsB amino acid sequence depicted in FIG. 4B. In some cases, the TnsB polypeptide has a length of from about 500 amino acids to about 600 amino acids (e.g., from about 500 amino acids (aa) to 525 aa, from 525 aa to 550 aa, from 550 aa to 575 aa, or from 575 aa to 600 aa). In some cases, the TnsB polypeptide has a length of 584 aa.

Non-limiting examples of other suitable TnsB polypeptides are provided in FIG. 6A-6E. For example, a TnsB polypeptide can comprise an amino acid sequence having at least 50%, at least 60%, at least 70%, at least 80%, at least 90%, at least 95%, at least 98%, at least 99%, or 100% amino acid sequence identity to any one of the TnsB polypeptide amino acid sequences depicted in FIG. 6A-6E.

A TnsC polypeptide can comprise an amino acid sequence having at least 50%, at least 60%, at least 70%, at least 80%, at least 90%, at least 95%, at least 98%, at least 99%, or 100% amino acid sequence identity to the S. hofmanni TnsC amino acid sequence depicted in FIG. 4C. A TnsC polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 200 amino acids to 276 amino acids (e.g., from about 200 amino acids (aa) to 225 aa, from 225 aa to 250 aa or from 250 aa to 276 aa) of the S. hofmanni TnsC amino acid sequence depicted in FIG. 4C. In some cases, the TnsC polypeptide has a length of from about 200 amino acids to 276 amino acids (e.g., from about 200 amino acids (aa) to 225 aa, from 225 aa to 250 aa or from 250 aa to 276 aa). In some cases, the TnsC polypeptide has a length of 276 aa.

Non-limiting examples of other suitable TnsC polypeptides are provided in FIG. 6K-6N. For example, a TnsC polypeptide can comprise an amino acid sequence having at least 50%, at least 60%, at least 70%, at least 80%, at least 90%, at least 95%, at least 98%, at least 99%, or 100% amino acid sequence identity to any one of the TnsC polypeptide amino acid sequences depicted in FIG. 6K-6N.

A TniQ polypeptide can comprise an amino acid sequence having at least 50%, at least 60%, at least 70%, at least 80%, at least 90%, at least 95%, at least 98%, at least 99%, or 100% amino acid sequence identity to the S. hofmanni TniQ amino acid sequence depicted in FIG. 4D. A TniQ polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 100 amino acids to 167 amino acids (e.g., from 100 amino acids (aa) to 125 aa, from 125 aa to 150 aa, or from 150 aa to 167 aa) of the S. hofmanni TniQ amino acid sequence depicted in FIG. 4D. In some cases, the TniQ polypeptide has a length of from about 100 amino acids to 167 amino acids (e.g., from 100 amino acids (aa) to 125 aa, from 125 aa to 150 aa, or from 150 aa to 167 aa). In some cases, the TniQ polypeptide has a length of 167 amino acids.

Non-limiting examples of other suitable TniQ polypeptides are provided in FIG. 6O-6R. For example, a TniQ polypeptide can comprise an amino acid sequence having at least 50%, at least 60%, at least 70%, at least 80%, at least 90%, at least 95%, at least 98%, at least 99%, or 100% amino acid sequence identity to any one of the TniQ polypeptide amino acid sequences depicted in FIG. 6O-6R.

### VcCAST

In some cases, a CAST comprises: i) a Cas6 polypeptide; ii) a Cas7 polypeptide; iii) a Cas8 polypeptide; iv) a TnsA polypeptide; v) a TnsB polypeptide; vi) a TnsC polypeptide; and vii) a TniQ polypeptide. An example of such a CAST is a Vibrio cholerae CAST (VcCAST).

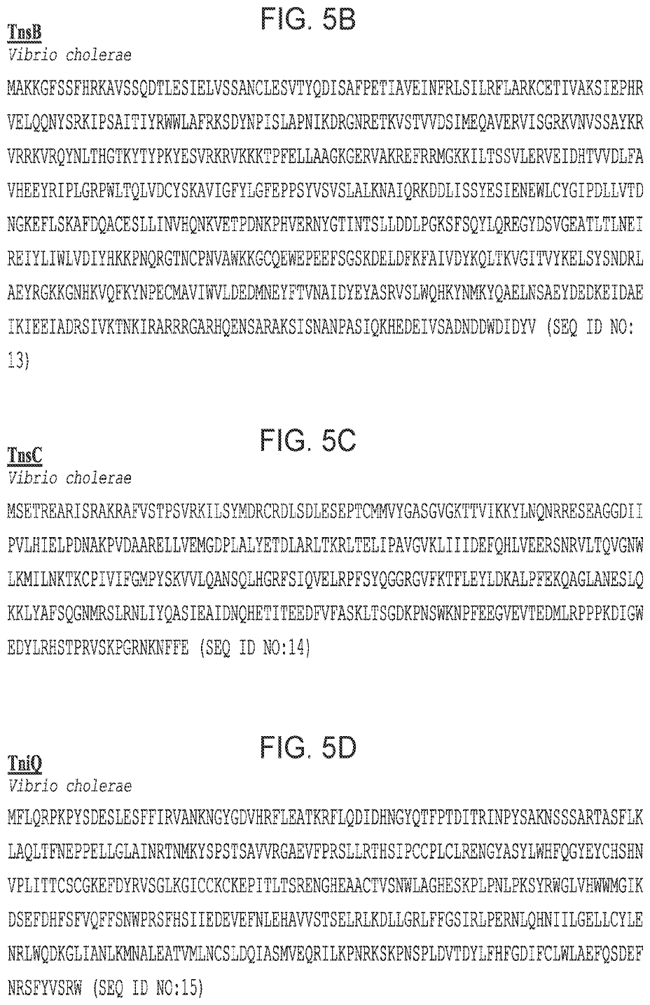

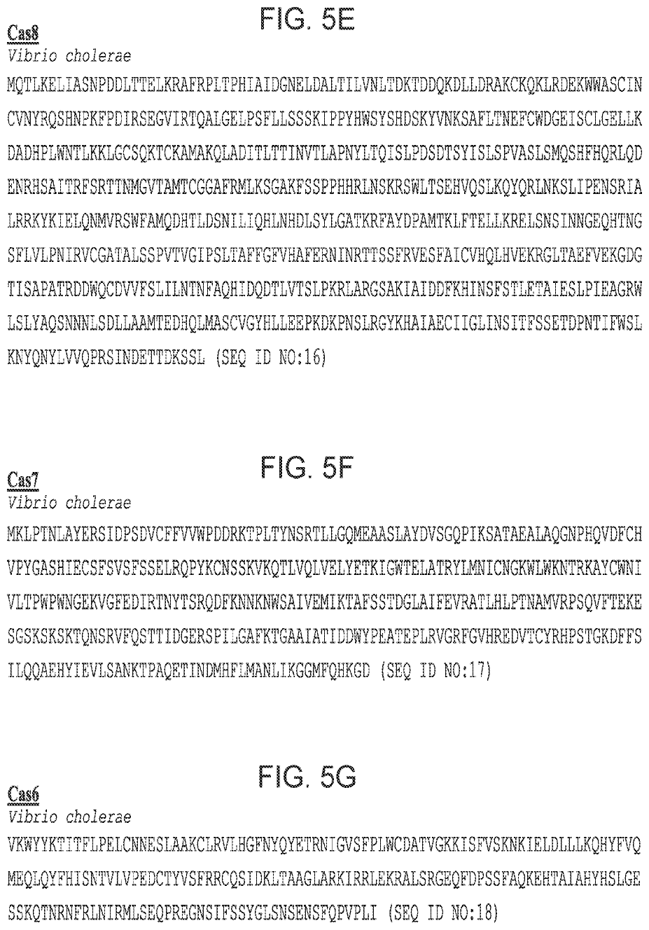

A Cas6 polypeptide can comprise an amino acid sequence having at least 50%, at least 60%, at least 70%, at least 80%, at least 90%, at least 95%, at least 98%, at least 99%, or 100% amino acid sequence identity to the V. cholera Cas6 polypeptide amino acid sequence depicted in FIG. 5G. A Cas6 polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 125 amino acids to 199 amino acids (e.g., from about 125 amino acids (aa) to 150 aa, from 150 aa to 175 aa, or from 175 aa to 199 aa) of the V. cholera Cas6 polypeptide amino acid sequence depicted in FIG. 5G. A Cas6 polypeptide can have a length of from about 125 amino acids to 199 amino acids (e.g., from about 125 amino acids (aa) to 150 aa, from 150 aa to 175 aa, or from 175 aa to 199 aa). A Cas6 polypeptide can have a length of 199 aa.

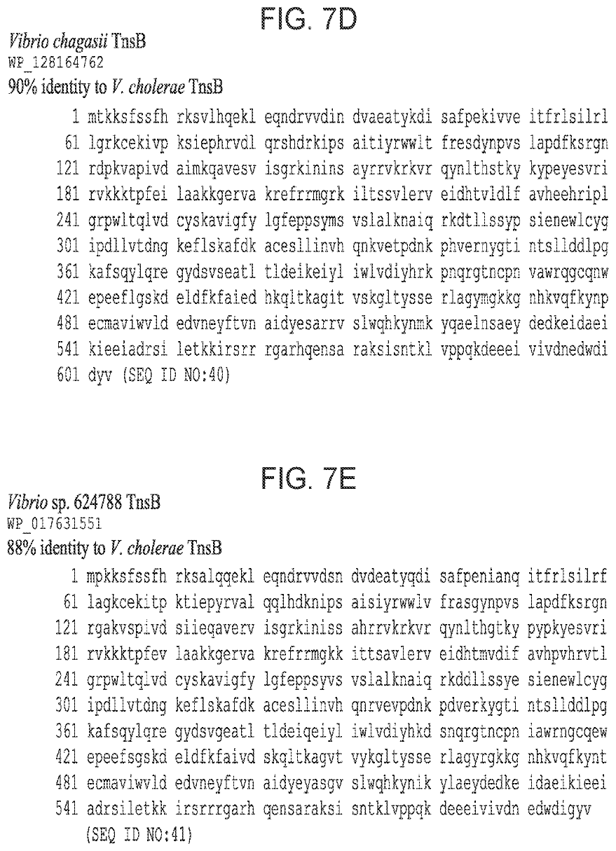

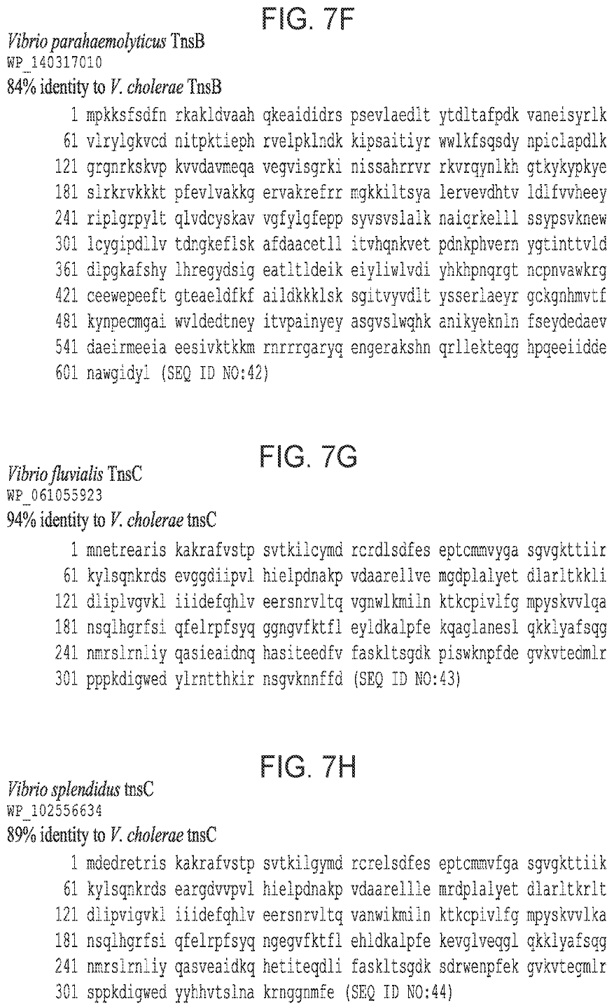

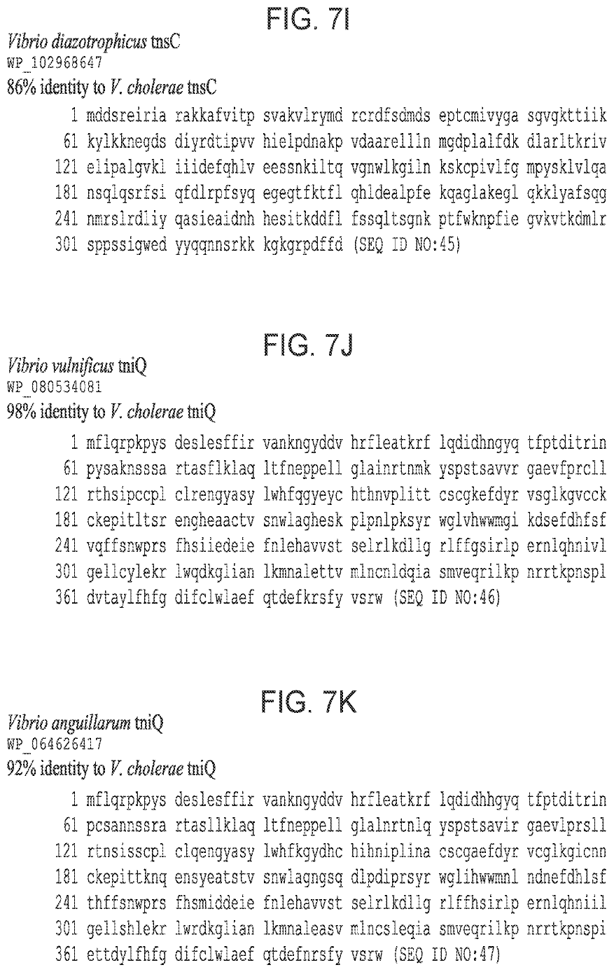

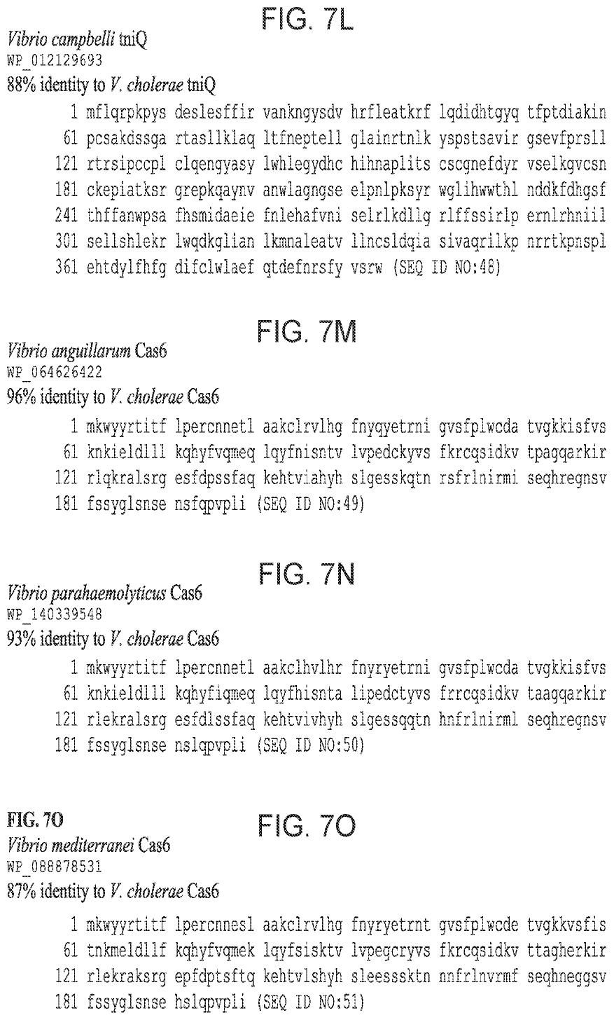

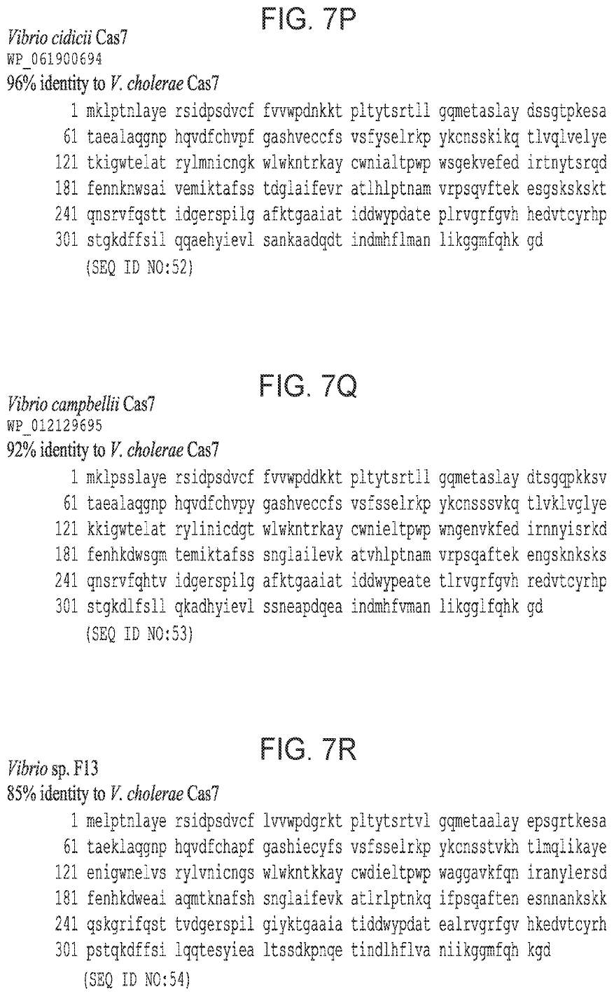

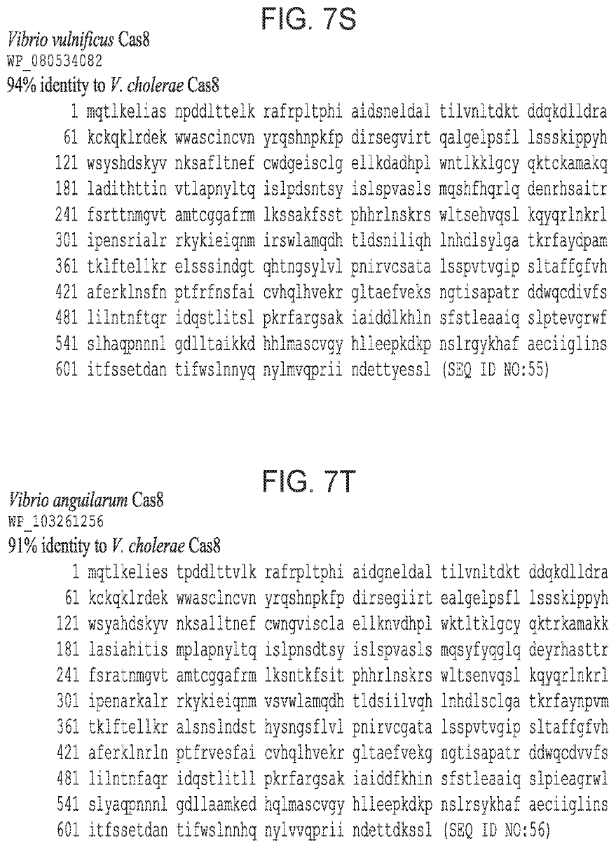

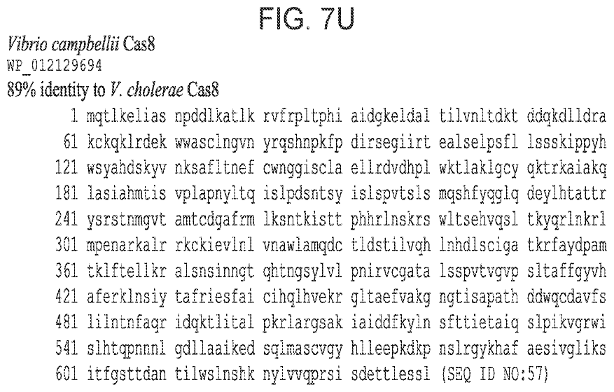

Non-limiting examples of other suitable Cas6 polypeptides are provided in FIG. 7M-7O. For example, a Cas6 polypeptide can comprise an amino acid sequence having at least 50%, at least 60%, at least 70%, at least 80%, at least 90%, at least 95%, at least 98%, at least 99%, or 100% amino acid sequence identity to any one of the Cas6 polypeptide amino acid sequences depicted in FIG. 7M-7O.

A Cas7 polypeptide can comprise an amino acid sequence having at least 50%, at least 60%, at least 70%, at least 80%, at least 90%, at least 95%, at least 98%, at least 99%, or 100% amino acid sequence identity to the V. cholera Cas7 polypeptide amino acid sequence depicted in FIG. 5F. A Cas7 polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 275 amino acids to 352 amino acids (e.g., from about 275 amino acids (aa) to 300 aa, from 300 aa to 325 aa, or from 325 aa to 352 aa) of the V. cholerae Cas7 polypeptide amino acid sequence depicted in FIG. 5F. A Cas7 polypeptide can have a length of from about 275 amino acids to 352 amino acids (e.g., from about 275 amino acids (aa) to 300 aa, from 300 aa to 325 aa, or from 325 aa to 352 aa). A Cas7 polypeptide can have a length of 352 aa.

Non-limiting examples of other suitable Cas7 polypeptides are provided in FIG. 7P-7R. For example, a Cas7 polypeptide can comprise an amino acid sequence having at least 50%, at least 60%, at least 70%, at least 80%, at least 90%, at least 95%, at least 98%, at least 99%, or 100% amino acid sequence identity to any one of the Cas7 polypeptide amino acid sequences depicted in FIG. 7P-7R.

A Cas8 polypeptide can comprise an amino acid sequence having at least 50%, at least 60%, at least 70%, at least 80%, at least 90%, at least 95%, at least 98%, at least 99%, or 100% amino acid sequence identity to the V. cholera Cas8 polypeptide amino acid sequence depicted in FIG. 5E. A Cas8 polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 575 amino acids to 640 amino acids (e.g., from about 575 amino acids (aa) to 600 aa, from 600 aa to 625 aa, or from 625 aa to 640 aa) of the V. cholerae Cas8 polypeptide amino acid sequence depicted in FIG. 5E. A Cas8 polypeptide can have a length of from about 575 amino acids to 640 amino acids (e.g., from about 575 amino acids (aa) to 600 aa, from 600 aa to 625 aa, or from 625 aa to 640 aa). A Cas8 polypeptide can have a length of 640 aa.

Non-limiting examples of other suitable Cas8 polypeptides are provided in FIG. 7S-7U. For example, a Cas8 polypeptide can comprise an amino acid sequence having at least 50%, at least 60%, at least 70%, at least 80%, at least 90%, at least 95%, at least 98%, at least 99%, or 100% amino acid sequence identity to any one of the Cas8 polypeptide amino acid sequences depicted in FIG. 7S-7U.

A tnsA polypeptide can comprise an amino acid sequence having at least 50%, at least 60%, at least 70%, at least 80%, at least 90%, at least 95%, at least 98%, at least 99%, or 100% amino acid sequence identity to the V. cholera tnsA polypeptide amino acid sequence depicted in FIG. 5A. A tnsA polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 150 amino acids to 222 amino acids (e.g., from about 150 amino acids (aa) to 175 aa, from 175 aa to 200 aa, or from 200 aa to 222 aa) of the tnsA amino acid sequence depicted in FIG. 5A. A tnsA polypeptide can have a length of from about 150 amino acids to 222 amino acids (e.g., from about 150 amino acids (aa) to 175 aa, from 175 aa to 200 aa, or from 200 aa to 222 aa). A tnsA polypeptide can have a length of 222 amino acids.

Non-limiting examples of other suitable tnsA polypeptides are provided in FIG. 7A-7C. For example, a tnsA polypeptide can comprise an amino acid sequence having at least 50%, at least 60%, at least 70%, at least 80%, at least 90%, at least 95%, at least 98%, at least 99%, or 100% amino acid sequence identity to any one of the tnsA polypeptide amino acid sequences depicted in FIG. 7A-7C.

A tnsB polypeptide can comprise an amino acid sequence having at least 50%, at least 60%, at least 70%, at least 80%, at least 90%, at least 95%, at least 98%, at least 99%, or 100% amino acid sequence identity to the V. cholera tnsB polypeptide amino acid sequence depicted in FIG. 5B. A tnsB polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 525 amino acids to 603 amino acids (e.g., from about 525 amino acids (aa) to 550 aa, from 550 aa to 575 aa, or from 575 aa to 603 aa) of the tnsB amino acid sequence depicted in FIG. 5B. A tnsB polypeptide can have a length of from about from about 525 amino acids to 603 amino acids (e.g., from about 525 amino acids (aa) to 550 aa, from 550 aa to 575 aa, or from 575 aa to 603 aa). A tnsB polypeptide can have a length of 603 amino acids.

Non-limiting examples of other suitable tnsB polypeptides are provided in FIG. 7D-7F. For example, a tnsB polypeptide can comprise an amino acid sequence having at least 50%, at least 60%, at least 70%, at least 80%, at least 90%, at least 95%, at least 98%, at least 99%, or 100% amino acid sequence identity to any one of the tnsB polypeptide amino acid sequences depicted in FIG. 7D-7F.

A tnsC polypeptide can comprise an amino acid sequence having at least 50%, at least 60%, at least 70%, at least 80%, at least 90%, at least 95%, at least 98%, at least 99%, or 100% amino acid sequence identity to the V. cholera tnsC polypeptide amino acid sequence depicted in FIG. 5C. A tnsC polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 225 amino acids to 330 amino acids (e.g., from about 225 amino acids (aa) to 250 aa, from 250 aa to 300 aa, or from 300 aa to 330 aa) of the tnsC amino acid sequence depicted in FIG. 5C. A tnsC polypeptide can have a length of from about 225 amino acids to 330 amino acids (e.g., from about 225 amino acids (aa) to 250 aa, from 250 aa to 300 aa, or from 300 aa to 330 aa). A tnsC polypeptide can have a length of 330 amino acids.

Non-limiting examples of other suitable tnsC polypeptides are provided in FIG. 7G-7I. For example, a tnsC polypeptide can comprise an amino acid sequence having at least 50%, at least 60%, at least 70%, at least 80%, at least 90%, at least 95%, at least 98%, at least 99%, or 100% amino acid sequence identity to any one of the tnsC polypeptide amino acid sequences depicted in FIG. 7G-7I.

A tniQ polypeptide can comprise an amino acid sequence having at least 50%, at least 60%, at least 70%, at least 80%, at least 90%, at least 95%, at least 98%, at least 99%, or 100% amino acid sequence identity to the V. cholera tniQ polypeptide amino acid sequence depicted in FIG. 5D. A tniQ polypeptide can comprise an amino acid sequence having at least 50%, at least 60%, at least 70%, at least 80%, at least 90%, at least 95%, at least 98%, at least 99%, or 100% amino acid sequence identity to a contiguous stretch of from about 300 amino acids to 394 amino acids (e.g., from about 300 amino acids (aa) to 325 aa, from 325 aa to 350 aa, from 350 aa to 375 aa, or from 375 aa to 394 aa) of the tniQ amino acid sequence depicted in FIG. 5D. A tniQ polypeptide can have a length of from about 300 amino acids to 394 amino acids (e.g., from about 300 amino acids (aa) to 325 aa, from 325 aa to 350 aa, from 350 aa to 375 aa, or from 375 aa to 394 aa). A tniQ polypeptide can have a length of 394 amino acids.

Non-limiting examples of other suitable tniQ polypeptides are provided in FIG. 7J-7L. For example, a tniQ polypeptide can comprise an amino acid sequence having at least 50%, at least 60%, at least 70%, at least 80%, at least 90%, at least 95%, at least 98%, at least 99%, or 100% amino acid sequence identity to any one of the tniQ polypeptide amino acid sequences depicted in FIG. 7J-7L.

### Promoters

The nucleotide sequence encoding the CAST complex polypeptides and/or the nucleotide sequence encoding the guide RNA can be operably linked to a promoter that is functional in a prokaryotic cell. In some cases, the nucleotide sequence encoding the CAST complex polypeptides is operably linked to a first promoter; and the nucleotide sequence encoding the guide RNA is operably linked to a second promoter. In some cases, the nucleotide sequence encoding the CAST complex polypeptides and the nucleotide sequence encoding the guide RNA are operably linked to the same promoter.

Suitable promoters include, constitutive promoters and inducible promoters. Inducible promoters include sugar-inducible promoters (e.g., lactose-inducible promoters; arabinose-inducible promoters); amino acid-inducible promoters; alcohol-inducible promoters; and the like. Suitable promoters include, e.g., lactose-regulated systems (e.g., lactose operon systems, sugar-regulated systems, isopropyl-beta-D-thiogalactopyranoside (IPTG) inducible systems, arabinose regulated systems (e.g., arabinose operon systems, e.g., an ARA operon promoter, pBAD, pARA, portions thereof, combinations thereof and the like), synthetic amino acid regulated systems, fructose repressors, a tac promoter/operator (pTac), tryptophan promoters, PhoA promoters, recA promoters, proU promoters, cst-1 promoters, tetA promoters, cadA promoters, nar promoters, P_{L} promoters, cspA promoters, and the like, or combinations thereof. In certain cases, a promoter comprises a Lac-Z, or portions thereof. In some cases, a promoter comprises a Lac operon, or portions thereof. In some cases, an inducible promoter comprises an ARA operon promoter, or portions thereof. In certain embodiments an inducible promoter comprises an arabinose promoter or portions thereof. An arabinose promoter can be obtained from any suitable bacteria. In some cases, an inducible promoter comprises an arabinose operon of E. coli or B. subtilis. In some cases, an inducible promoter is activated by the presence of a sugar or an analog thereof. Non-limiting examples of sugars and sugar analogs include lactose, arabinose (e.g., L-arabinose), glucose, sucrose, fructose, IPTG, and the like. Suitable promoters include a T7 promoter; a pBAD promoter; a lacIQ promoter; and the like. In some cases, the promoter is a J23119 promoter. Many bacterial promoters are known in the art; bacterial promoters can be found on the internet at parts(dot)igem(dot)org/promoters.

### Transposons

A transposon suitable for inclusion in a nucleic acid construct of a system of the present disclosure can have a length of up to about 100 kilobases (kb). For example, a transposon can have a length of from 0.1 kb to 0.5 kb, from 0.5 kb to 1 kb, from 1 kb to 5 kb, from 5 kb to 10 kb, from 10 kb to 15 kb, from 15 kb to 20 kb, from 20 kb to 25 kb, from 25 kb to 30 kb, from 30 kb to 35 kb, from 35 kb to 40 kb, from 40 kb to 45 kb, from 45 kb to 50 kb, from 50 kb to 55 kb, from 55 kb to 60 kb, from 60 kb to 65 kb, from 65 kb to 70 kb, from 70 kb to 75 kb, from 75 kb to 80 kb, from 80 kb to 85 kb, from 85 kb to 90 kb, from 90 kb to 95 kb, or from 95 kb to 100 kb.

A transposon suitable for inclusion in a nucleic acid construct of a system of the present disclosure can comprise one or more of: a) one or more nucleotide sequences encoding one or more polypeptides that confer on a prokaryotic cell resistance to one or more antibiotics; b) one or more nucleotide sequences encoding one or more enzymes in a biosynthetic pathway; c) one or more nucleotide sequences encoding one or more enzymes in a carbon utilization pathway (e.g., a polysaccharide utilization pathway); d) one or more nucleotide sequences encoding one or more polypeptides comprising a light-oxygen-voltage-sensing domain (LOV domain); e) a screenable marker (a detectable polypeptide; e.g., a polypeptide that provides a detectable signal such as a fluorescent signal); f) a polypeptide that provides for detection of an analyte in a bacterium; g) one or more nucleotide sequences encoding one or more therapeutic polypeptides; h) one or more nucleotide sequences encoding one or more nutritional polypeptides; i) one or more nucleotide sequences encoding one or more polypeptides that confer antibiotic sensitivity on a target prokaryotic cell; j) one or more nucleotide sequences encoding one or more polypeptides that facilitate isolation of a target prokaryotic cell; k) one or more nucleotide sequences encoding one or more enzymes in a nitrogen utilization pathway; 1) one or more nucleotide sequences encoding one or more enzymes in a sulfur utilization pathway; m) one or more nucleotide sequences encoding one or more enzymes that degrade an allergen; n) one or more nucleotide sequences encoding one or more polypeptides that confers resistance to a phage (e.g., a bacteriophage); o) one or more nucleotide sequences encoding one or more polypeptides that provide for mobility of a gene edit; and the like. A transposon can include one or more nucleotide sequences encoding one or more polypeptides that allow establishment of a unique metabolic niche.

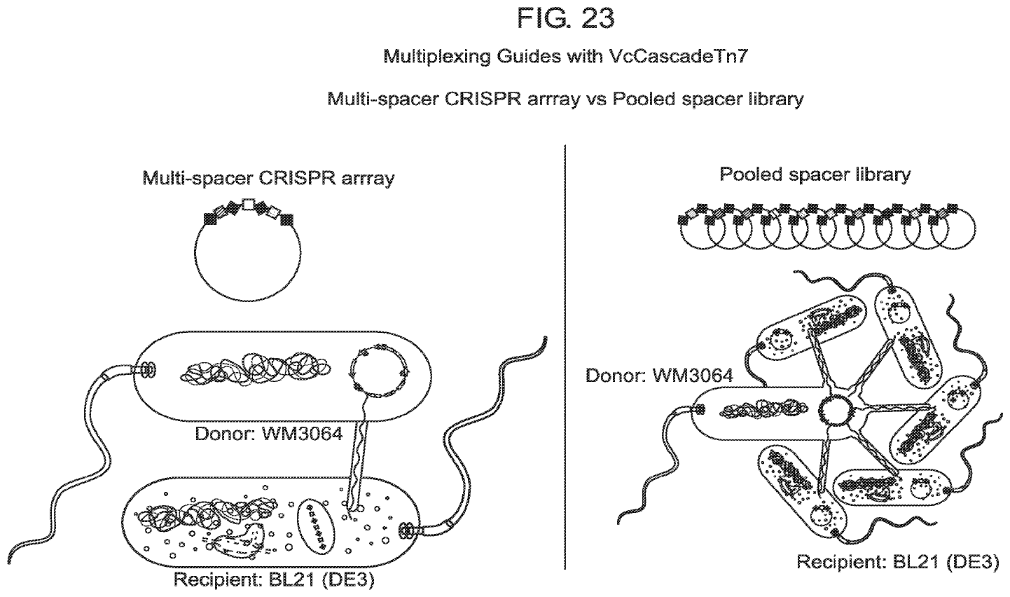

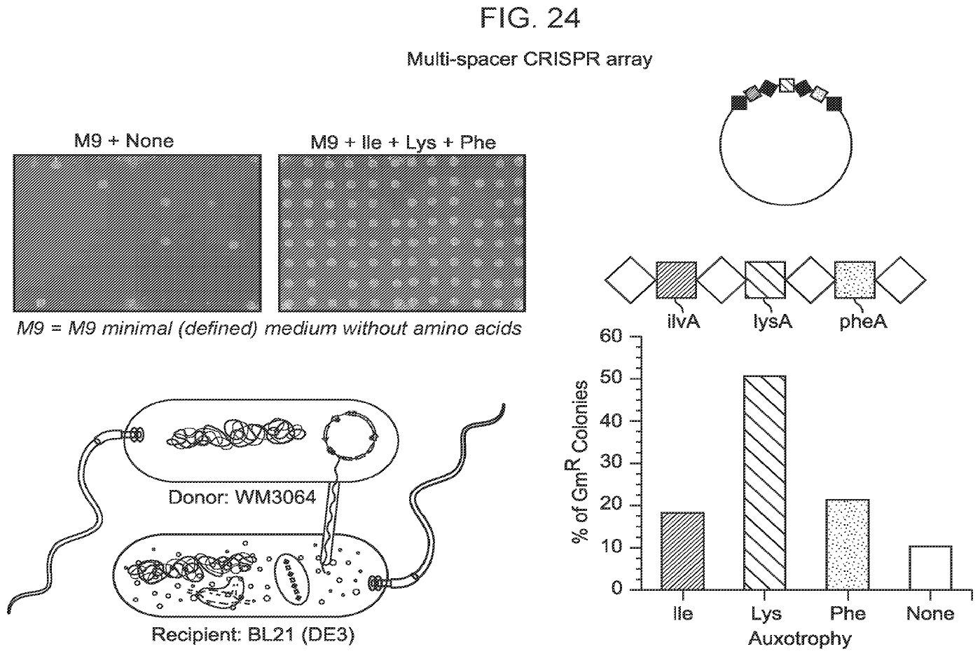

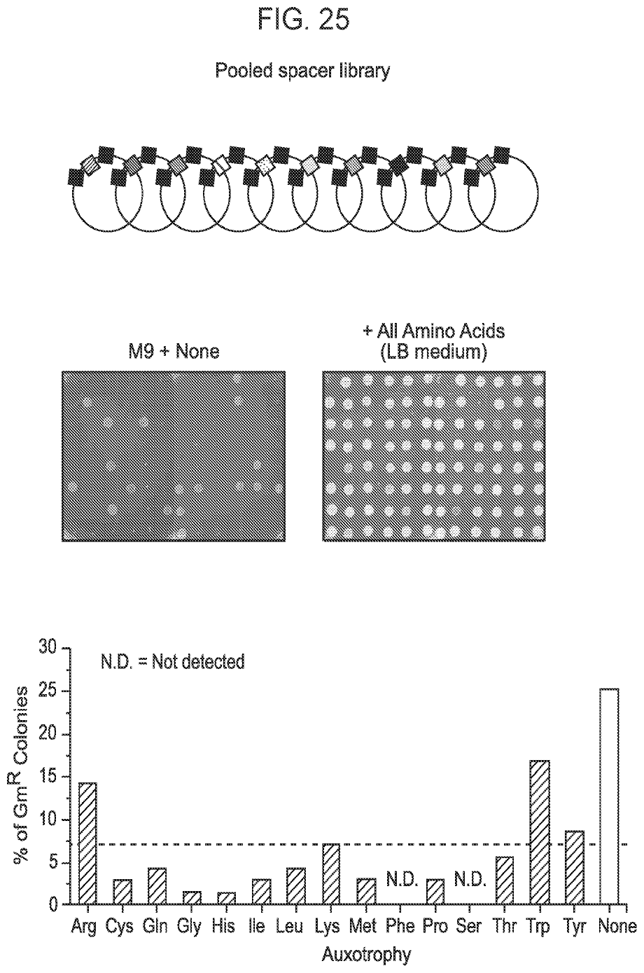

A transposon can function to knock out an endogenous nucleic acid in a target bacterium, e.g., to delete all or a portion of an endogenous nucleic acid in a target prokaryotic cell or to introduce a loss-of-function mutation in an endogenous nucleic acid in a target prokaryotic cell. A “knockout” includes deletion of all or a portion of a nucleic acid; and includes introduction of a loss-of-function mutation in a nucleic acid. For example, a transposon can function to delete all or a portion of an endogenous nucleic acid in a target prokaryotic cell (e.g., target bacterium; target archaeon), or to introduce a loss-of-function mutation in an endogenous nucleic acid in a target prokaryotic cell, where the endogenous nucleic acid comprises one or more nucleotide sequences encoding one or more polypeptides that confer on a prokaryotic cell resistance to one or more antibiotics. A transposon can function to generate an auxotroph, e.g., an amino acid auxotroph (see, e.g., FIG. 23 to FIG. 25). A transposon can function to knock out an essential gene (e.g., a nucleic acid encoding one or more polypeptides that are essential to cell survival, cell proliferation, cell metabolism, etc.). A transposon can function to knock out a nucleic acid encoding a toxin. A transposon can function to knock out a counter-selectable gene, or a gene that confers a fitness advantage in a certain growth condition or medium composition (e.g., a galK knockout can grow in presence of 2-deoxygalactose; a pyrF knockout can grow in presence of 5-fluoroorotic acid; a thyA knockout can grow in presence of trimethoprim; etc.)

A transposon can comprise one or more nucleotide sequences encoding one or more polypeptides that confer resistance to one or more antibiotics in a target prokaryotic cell.

A transposon can comprise: a) one or more nucleotide sequences encoding magnetosome biosynthetic pathway polypeptides; b) one or more nucleotide sequences encoding gas vesicle biosynthetic polypeptides; c) one or more nucleotide sequences encoding one or more polypeptides in a porphyrin polysaccharide utilization pathway; d) one or more nucleotide sequences encoding one or more polypeptides in a glycosaminoglycan utilization pathway; e) one or more nucleotide sequences encoding one or more polypeptides in a glycosaminoglycan utilization pathway; f) one or more nucleotide sequences encoding one or more polypeptides in a non-caloric artificial sweetener utilization pathway; f) one or more nucleotide sequences encoding one or more polypeptides in a B-vitamin biosynthetic pathway; g) one or more nucleotide sequences encoding one or more polypeptides in an ethanolamine utilization pathway; h) one or more nucleotide sequences encoding one or more polypeptides in a sucrose utilization pathway; i) one or more nucleotide sequences encoding one or more polypeptides in a mevalonate biosynthetic pathway; j) one or more nucleotide sequences encoding one or more polypeptides in a polyketide biosynthetic pathway; and the like.

A transposon can comprise one or more nucleotide sequences encoding one or more polypeptides that provide for isolation of a target prokaryotic cell; e.g., a FLASH tag; FAST; iLOV; phiLOV; smURFP, IFP2.0; evoglow-Pp1; UnaG; a SNAP tag; a CLIP tag; a Halo tag; a spinach aptamer; mango aptamer; and the like. See, e.g., Thorn (2017) Mol. Biol. Cell 28:848; and Wang et al. (2017) Mol. Bhiochem. Parasitol. 216:1. A transposon can comprise one or more nucleotide sequences encoding one or more polypeptides fluorescent proteins or tags that are detectable in anaerobic conditions, such as an anaerobic green fluorescent protein (GFP); see, e.g., Landete et al. ((2015) App. Microbiol. Biotechnol. 99:6865) and Streett et al. (2019) Appl. Environmental Microbiol. 85:e00622. Tagging surface exposed proteins with FLAG tag, His tag, Myc tag and the like, to be immunolabeled with fluorescence/magnetic-conjugated antibodies. Also suitable are tetracysteine tags to enable staining with biarsenical dyes (e.g., for staining with FlAsH and ReAsH dyes).

A transposon can comprise a nucleotide sequence encoding a fluorescent polypeptide. Suitable fluorescent proteins include, but are not limited to, green fluorescent protein (GFP) and variants thereof, blue fluorescent protein (BFP), cyan fluorescent protein (CFP), yellow fluorescent protein (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. See, e.g., Thorn (2017) Mol. Biol. Cell 28:848.

### CAST-Recognition Sites

As noted above, a transposon system of the present disclosure comprises a transposon or an insertion site for a transposon, where the transposon or the insertion site for a transposon is flanked by recognition sites (nucleotide sequences) that are bound by and cleaved by a CAST complex. The recognition sites are referred to as “left end” and “right end.” Recognition sites bound by and cleaved by a CAST complex are known in the art.

For example, “left end” and “right end” recognition sites bound by and cleaved by a VcCAST are:

(left end; SEQ ID NO: 1)
TGTTGATGCAACCATAAAGTGATATTTAATAATTATTTATAATCAGCAAC
TTAACCACAAAACAACCATATATTGATATCTCACAAAACAACCATAAGTT
GATATTTT;
and
(right end; SEQ ID NO: 2)
GCAATATCAATTTATGGGTGTGATAATTATCAATTTATGGGTGTAATTAT
CATTTTATGGTTGTATCAACA.

As another example, “left end” and “right end” recognition sites bound by and cleaved by an ShCAST are:

(left end; SEQ ID NO: 3)
TGTACAGTGACAAATTATCTGTCGTCGGTGACAGATTAATGTCATTGTGA
CTATTTAATTGTCGTCGTGACCCATCAGCGTTGCTTAATTAATTGATGAC
AAATTAAATGTCA;
and
(right end; SEQ ID NO: 4)
CGACAGTCAATTTGTCATTATGAAAATACACAAAAGCTTTTTCCTATCTT
GCAAAGCGACAGCTAATTTGTCACAATCACGGACAACGACATCTATTTTG
TCACTGCAAAGAGGTTATGCTAAAACTGCCAAAGCGCTATAATCTATACT
GTATAAGGATTTTACTGATGACAATAATTTGTCACAACGACATATAATTA
GTCACTGTACA.

### Guide RNA

As noted above, a transposon system of the present disclosure comprises a nucleotide sequence encoding one or more guide RNAs. The guide RNA comprises: i) a nucleotide sequence that hybridizes to a target nucleotide sequence in a prokaryotic genome; and ii) a nucleotide sequence that binds to a polypeptide in the CAST complex. For example, the guide RNA comprises: i) a targeter RNA that comprises a nucleotide sequence (“guide sequence”) that hybridizes to a target nucleotide sequence in a prokaryotic genome; and ii) an activator RNA that comprises a nucleotide sequence that binds to a polypeptide in the CAST complex. A CAST forms a complex with a guide RNA. A CAST/guide RNA complex directs a transposon to a genomic site complementary to a guide RNA. See, e.g., Klompe et al. (2019) Nature 571:219; and Peters et al. (2019) Mol. Microbiol. 112:1635.

In some cases, a transposon system of the present disclosure comprises a nucleotide sequence encoding a single guide RNA. In some cases, a transposon system of the present disclosure comprises nucleotide sequences encoding two or more guide RNAs, each guide RNA comprising a nucleotide sequence that hybridizes to a target nucleotide sequence in a prokaryotic cell genome. For example, in some cases, a transposon system of the present disclosure comprises nucleotide sequences encoding 2, 3, 4, or 5 (or more than 5) different guide RNAs, each targeted to a different target nucleic acid.

A nucleic acid that binds to a polypeptide in a CAST complex, forming a CAST/guide nucleic acid complex, and targets the CAST/guide nucleic acid to a specific target sequence within a target DNA (e.g., prokaryotic genome) is referred to herein as a “guide RNA.” It is to be understood that in some cases, a hybrid DNA/RNA can be made such that a guide RNA includes DNA bases in addition to RNA bases—but the term “guide RNA” is still used herein to encompass such hybrid molecules. A subject guide RNA includes a guide sequence (also referred to as a “spacer”)(that hybridizes to target sequence of a target DNA) and a constant region (e.g., a region that is adjacent to the guide sequence and binds to a polypeptide in the CAST complex). A “constant region” can also be referred to herein as a “protein-binding segment.”

The guide sequence has complementarity with (hybridizes to) a target sequence of the target DNA. In some cases, the guide sequence is 15-35 nucleotides (nt) in length (e.g., 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, 30-32, 28-32, or 18-22 nt in length). In some cases, the guide sequence is 18-24 nucleotides (nt) in length. In some cases, the guide sequence is at least 15 nt long (e.g., at least 16, 18, 20, or 22 nt long). In some cases, the guide sequence is at least 17 nt long. In some cases, the guide sequence is at least 18 nt long. In some cases, the guide sequence is at least 20 nt long. In some cases, the guide sequence is 32 nt long. In some cases, VcCAST guides are included in a CRISPR array (repeat-spacer-repeat). In some cases, a ShCAST guides includes a 23-nt target complementarity.

In some cases, the guide sequence has 80% or more (e.g., 85% or more, 90% or more, 95% or more, or 100% complementarity) with the target sequence of the target DNA. In some cases, the guide sequence is 100% complementary to the target sequence of the target DNA. In some cases, the target DNA includes at least 15 nucleotides (nt) of complementarity with the guide sequence of the guide RNA.

In some cases, the constant region of a guide RNA is 15 or more nucleotides (nt) in length (e.g., 18 or more, 20 or more, 21 or more, 22 or more, 23 or more, 24 or more, 25 or more, 26 or more, 27 or more, 28 or more, 29 or more, 30 or more, 31 or more nt, 32 or more, 33 or more, 34 or more, or 35 or more nt in length). In some cases, the constant region of a guide RNA is 18 or more nt in length.

In some cases, the guide RNA is a dual-molecule guide RNA. In some cases, the guide RNA is a single-molecule RNA (also referred to as a “single guide RNA” or “sgRNA”).

As an example, a crRNA for VcCAST system is

(SEQ ID NO: 5)
GTGAACTGCCGAGTAGGTAGCTGATAACGAGACCTCGTTTACCTATCGGT
CTCGTGAACTGCCGAGTAGGTAGCTGATAAC.

As an example, a sgRNA for the ShCas12k is

(SEQ ID NO: 6)
ATATTAATAGCGCCGCAATTCATGCTGCTTGCAGCCTCTGAATTTTGTTA
AATGAGGGTTAGTTTGACTGTATAAATACAGTCTTGCTTTCTGACCCTGG
TAGCTGCTCACCCTGATGCTGCTGTCAATAGACAGGATAGGTGCGCTCCC
AGCAATAAGGGCGCGGATGTACTGCTGTAGTGGCTACTGAATCACCCCCG
ATCAAGGGGGAACCCTAAATGGGTTGAAAGGGAGACCGAGATCTCGAGGT
CTCC.

### Exemplary Constructs

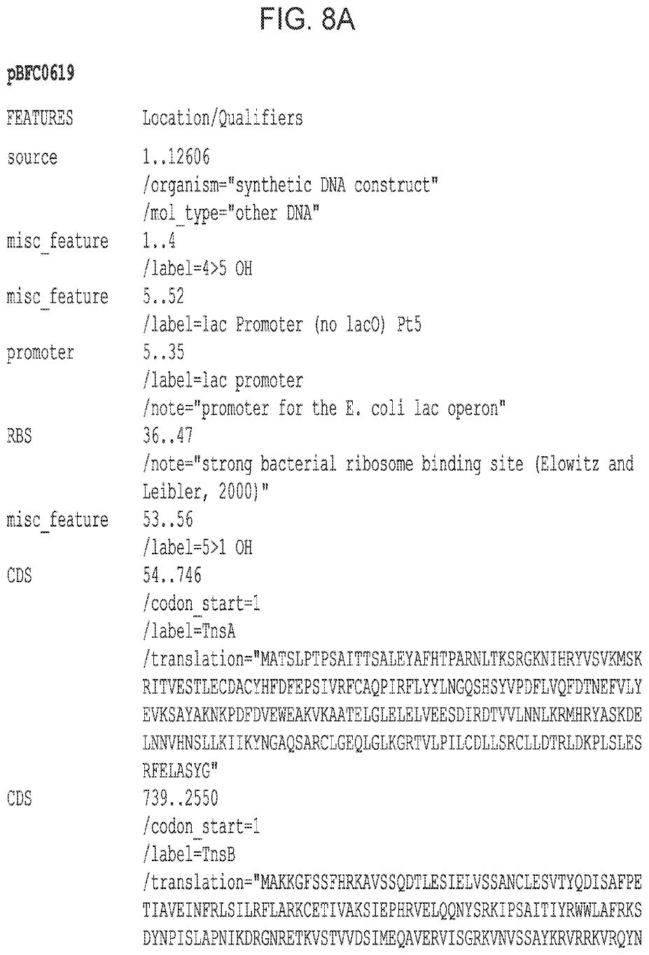

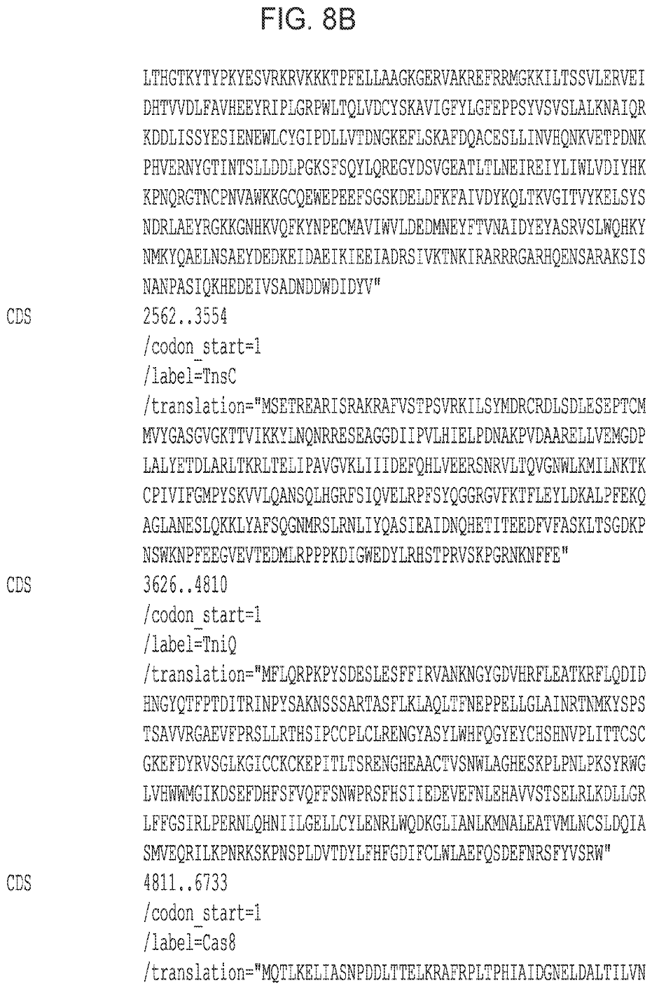

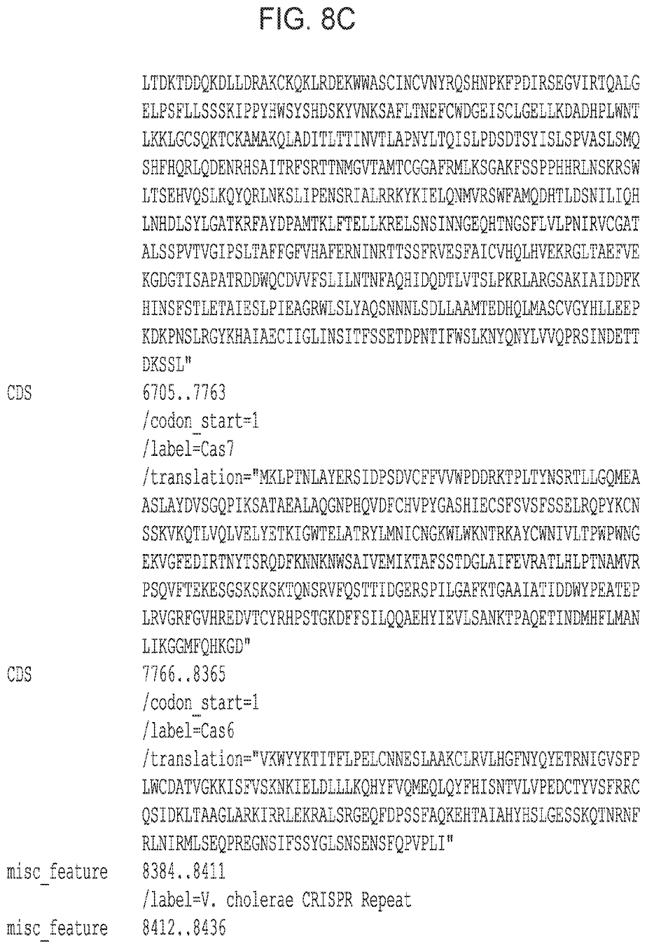

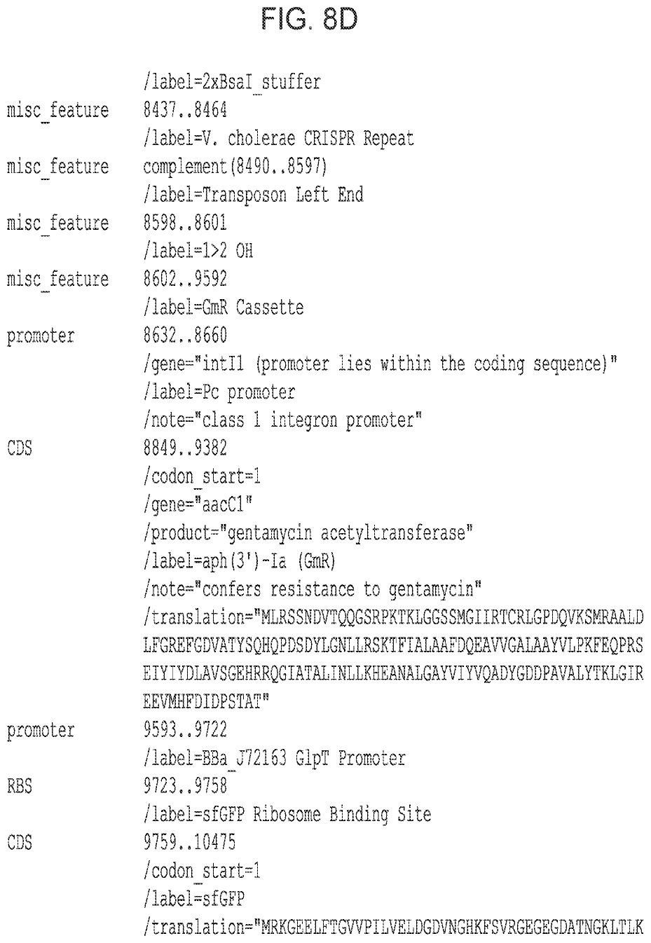

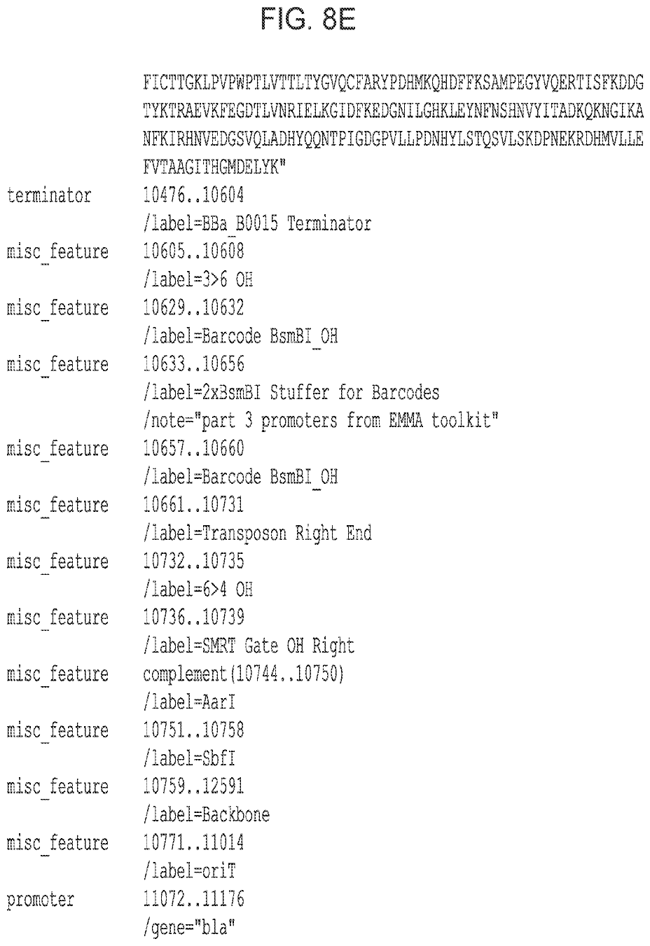

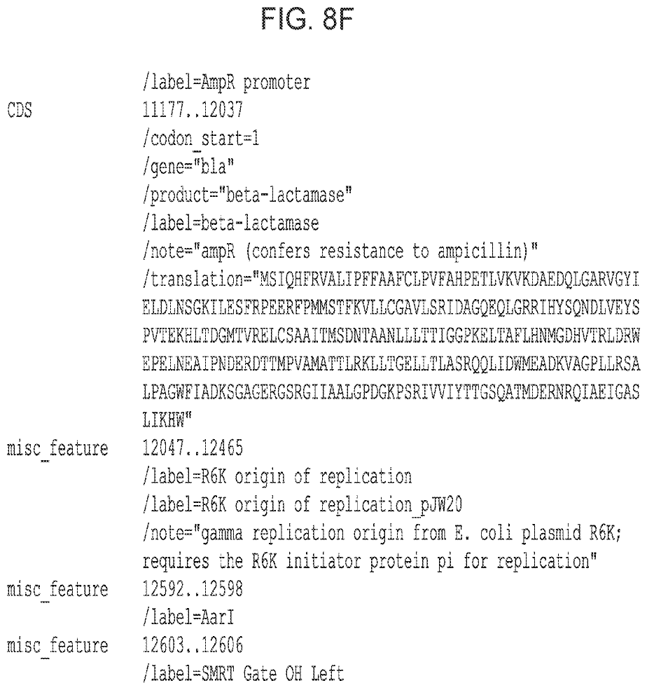

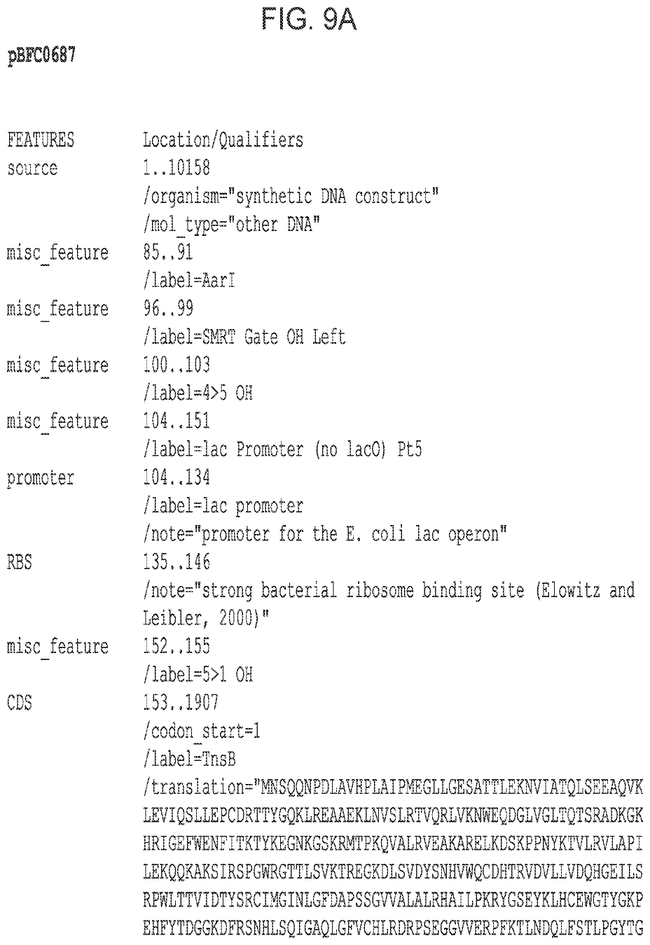

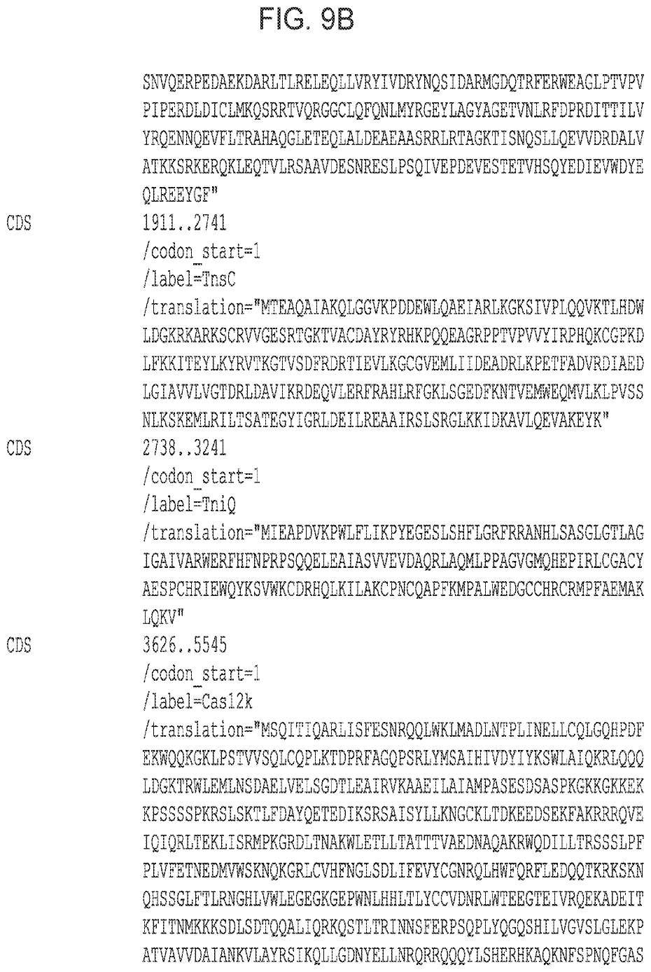

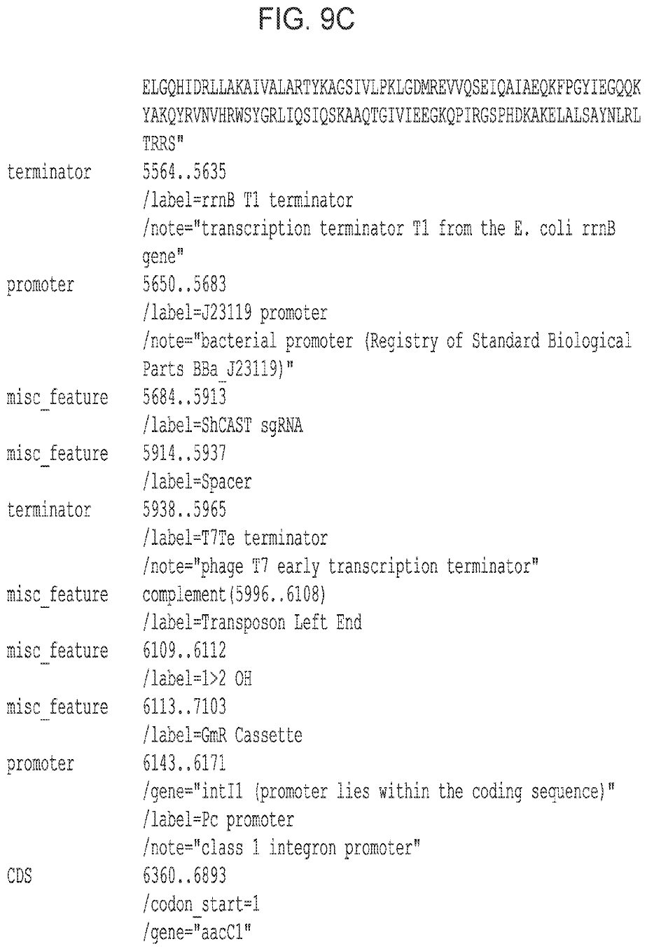

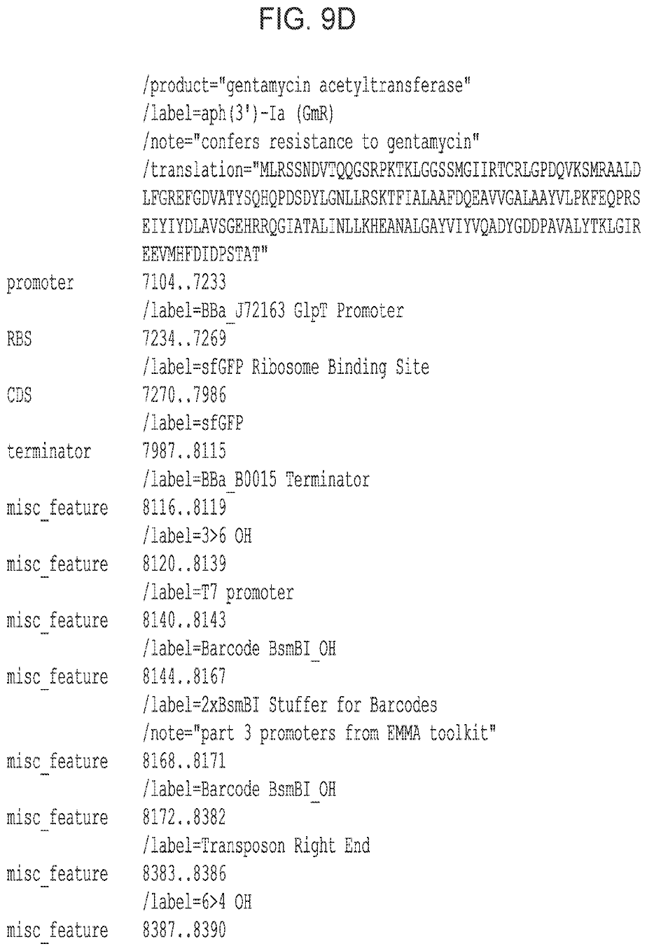

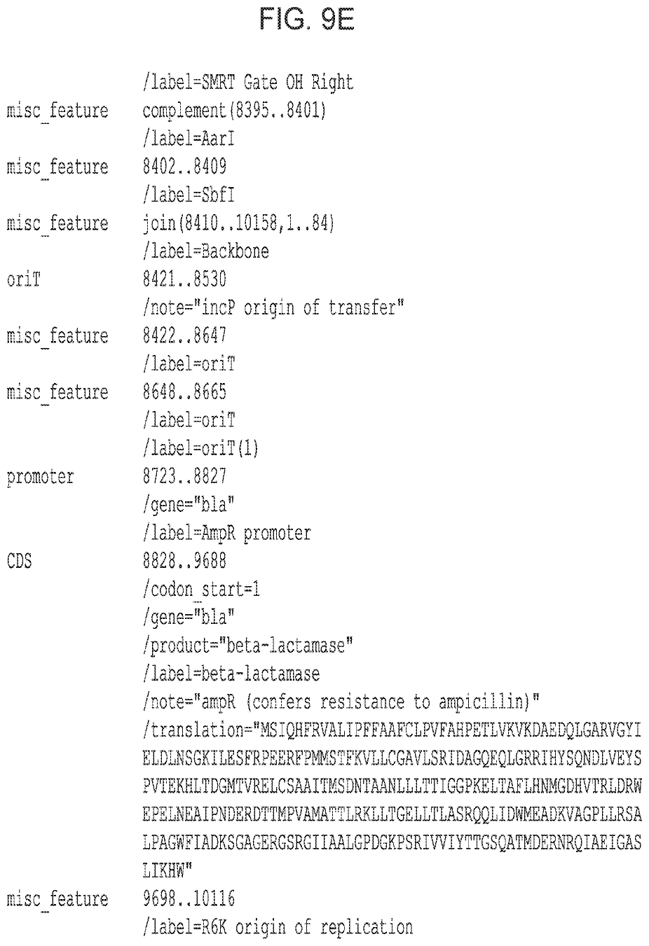

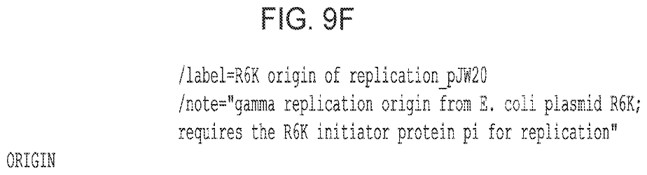

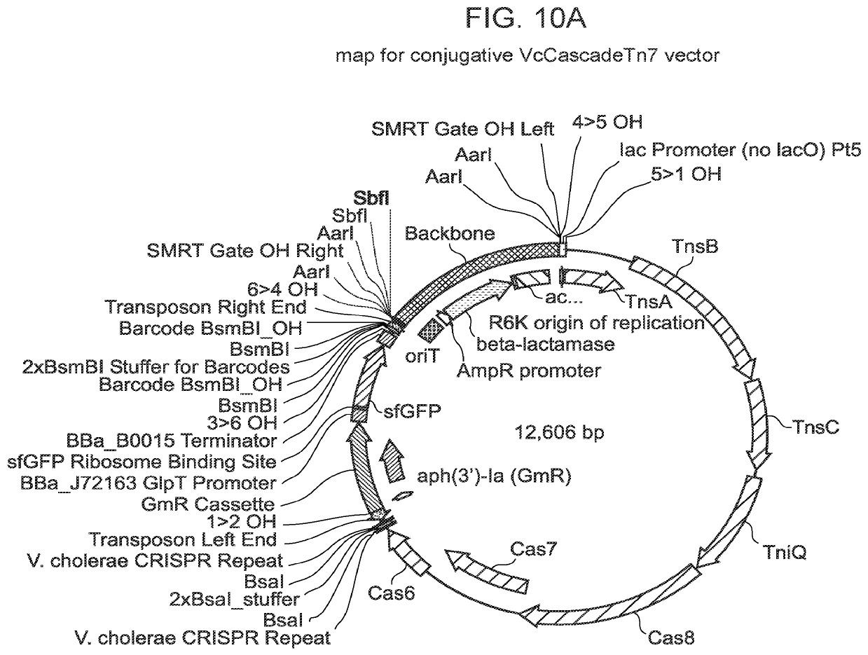

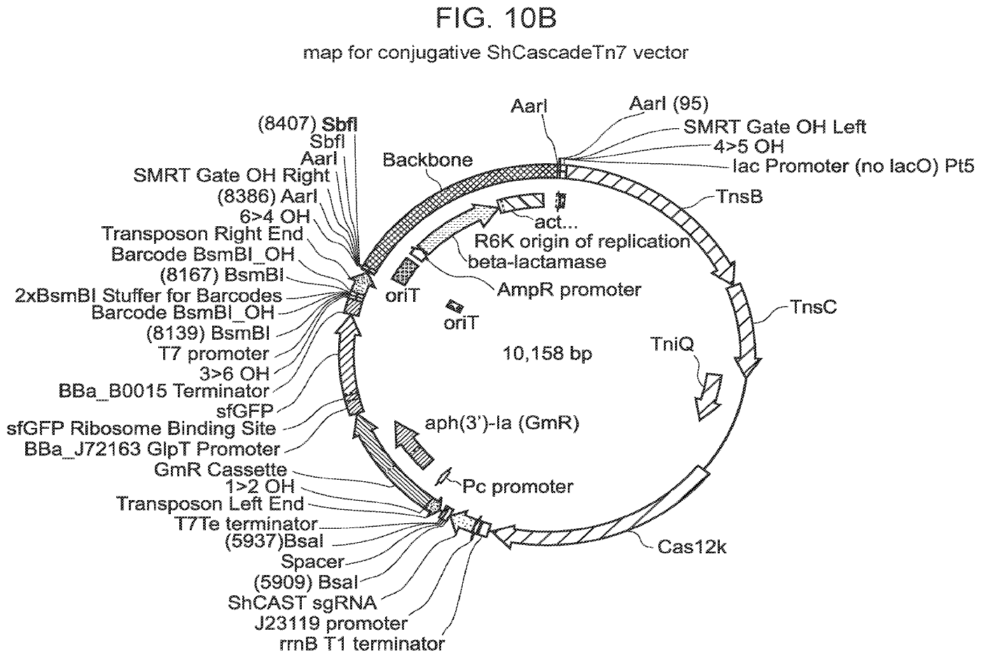

Exemplary single-construct conjugative transposon constructs of the present disclosure include pBFC0619, as illustrated in FIG. 8A-8E; and pBFC0687, as illustrated in FIG. 9A-9F. Maps for these constructs are presented in FIGS. 10A and 10B.

### Target Prokaryotic Cells

Target prokaryotic cells include bacteria and archaea. In some cases, the target prokaryotic cells are bacteria. In some cases, the target prokaryotic cells are archaea.

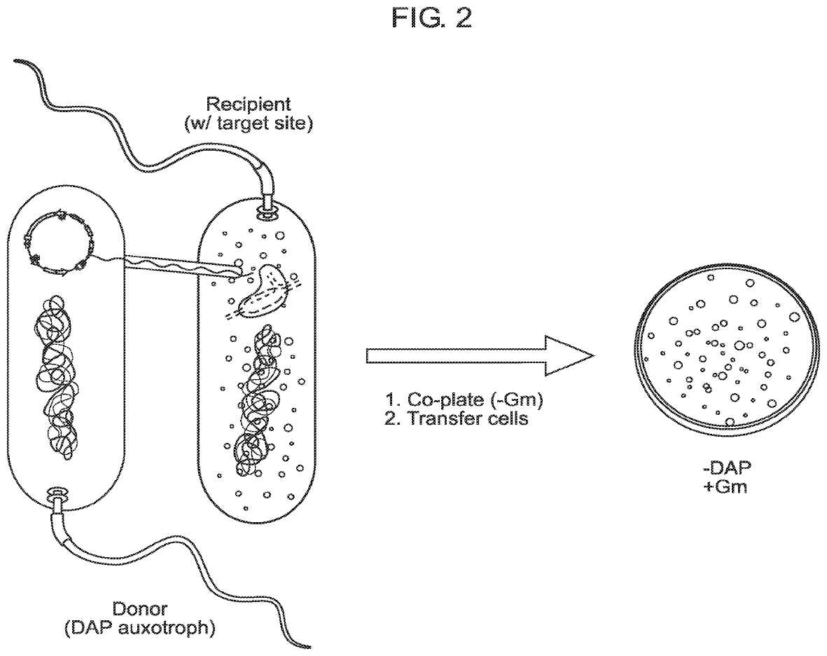

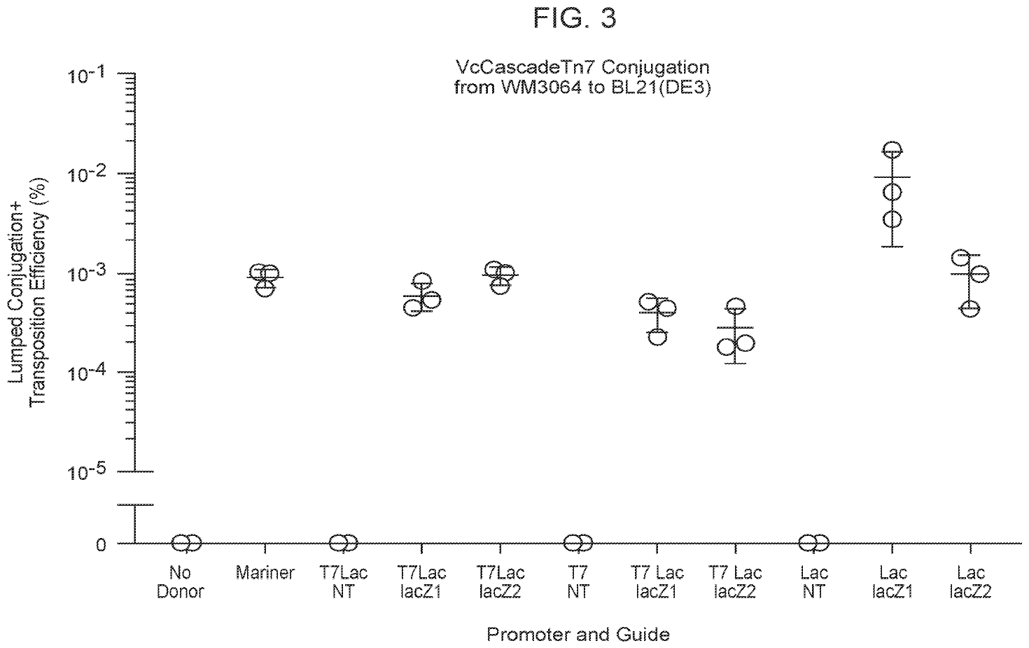

In some cases, target prokaryotic cells include bacteria and/or archaea that have not yet been cultured or isolated in a laboratory in monoculture. This would include most phyla of the candidate phyla radiation, most archaeal phyla, and numerous phyla of bacteria. See, e.g., FIG. 2 of Hug et al. (2016) Nature Microbiol. 1:16048.

Target prokaryotic cells include prokaryotic cells found in a natural environment such as the gastrointestinal tract of a mammal (e.g., a human); the microbiome of a human; the microbiome of a non-human animal soil; hot springs; oceans; marshland; swamps; etc. Target prokaryotic cells include prokaryotic cells found in wastewater, agricultural runoff, and the like. Target prokaryotic cells include prokaryotic cells involved in food processing (e.g., fermentations to produce beverages or food that rely on a mixed community of cells such as with kimchi, soy sauce, or kombucha). Target prokaryotic cells include prokaryotic cells present in the rhizosphere. Target prokaryotic cells include prokaryotic cells present on the plant surface microbiome (the plant microbiome). Target prokaryotic cells include prokaryotic cells found in industrial processes relying on communities of microorgansisms such as industrial wastewater treatment or bioreactors used for bioremediation of wastes (i.e. thiocyanate (SCN) degradation reactors used for gold mining runoff). Target prokaryotic cells include prokaryotic cells that find use in and/or are found in one or more of: the plant microbiome, food processing (e.g., wine, cheese, yogurt, etc.), bioremediation, and industrial processes.

Target bacteria include bacteria present in the human gastrointestinal tract. Target bacteria include bacteria of the phyla Firmicutes, Bacteroidetes, Actinobacteria, and Proteobacteria. Target bacteria include bacteria of the genera Lactobacillus, Bacteroides, Clostridum, Faecalibacterium, Eubacterium, Ruminococcus, Peptococcus, Roseburia, Peptostreptococcus, Bifidobacterium, Alistipes, Parabacteroides, Porphyromonas, Prevotella, Collinsalla, Escherichia, and Desulfovibrio. See, e.g., Rinninella et al. (2019) Microoganisms 7:14. Examples of target bacteria include, e.g., Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium. Staphylococcus epidermidis, Eubacterium limosum, Tissirella praeacuta, Fusobacterium mortiferum, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

Target bacteria include bacteria present in the gastrointestinal tract of an ungulate (e.g., a bovine; an equine; an ovine; a caprine; etc.).

Other target bacteria include, e.g., bacteria associated with nosocomial infections in humans. Other target bacteria include soil bacteria.

In some cases, a target prokaryotic cell is one that is refractory to genetic modification by electroporation. In some cases, a target prokaryotic cell is one that is refractory to genetic modification by chemically-induced competence (e.g., competence induced by calcium chloride, rubidium chloride, and the like). In some cases, a target prokaryotic cell is one that is refractory to genetic modification by heat shock. In some cases, a target prokaryotic cell is one that is refractory to natural transformation. In some cases, a target prokaryotic cell is one that is refractory to isolation. In some cases, a target prokaryotic cell is one that is refractory growth in monoculture (e.g., in an industrial setting, a research laboratory setting, or the like).

Archaea that are suitable target prokaryotic cells include, e.g., archaea any species in any of the phyla Aenigmarchaeota, Diapherotrites, Nanoarchaeota, Nanohaloarchaeota, Micrarchaeota, Pacearchaeota, Parvarchaeota, Woesearchaeota, Aigarchaeota, Bathyarchaeota, Crenarchaeota, Geoarchaeota, Korarchaeota, Thaumarchaeota, Lokiarchaeota, Thorarchaeota, Odinarchaeota, Heimdallarchaeota, and the like.

### Genetically Modified Prokaryotic Cells

The present disclosure provides a prokaryotic cell comprising a transposon system of the present disclosure. A prokaryotic cell of the present disclosure can be a “donor” bacterium, i.e., one that comprises a subject transposon system that is to be transferred to a target bacterium (a “recipient” bacterium). A prokaryotic cell of the present disclosure can be a “donor” archaeon, i.e., one that comprises a subject transposon system that is to be transferred to a target archaeon (a “recipient” archaeon).

The present disclosure also provides a genetically modified prokaryotic cell, where the genetically modified has been genetically modified by virtue of contact with a “donor” bacterium of the present disclosure; i.e., the genetically modified has been genetically modified with a transposon that is present in the transposon system present in the “donor” bacterium.

The present disclosure also provides a genetically modified prokaryotic cell, where the genetically modified has been genetically modified by virtue of contact with a “donor” archaeon of the present disclosure; i.e., the genetically modified has been genetically modified with a transposon that is present in the transposon system present in the “donor” archaeon.

The present disclosure provides a heterogeneous population of genetically modified prokaryotic cells, where the population comprises a plurality of genetically modified prokaryotic cells, which prokaryotic cells are the recipients of transposons present in a library of the present disclosure (e.g., are the recipients of a member of a library of the present disclosure). The heterogeneous population can comprise from 10 to 10⁹ different prokaryotic cells; e.g., from 10 to 10², from 10² to 10³, from 10³ to 10⁴, from 10⁴ to 10⁵, from 10⁵ to 10⁶, from 10⁶ to 10⁷, from 10⁷ to 10⁸, or from 10⁸ to 10⁹ different prokaryotic cells, which comprise different transposons from a library of the present disclosure. In some cases, the population of prokaryotic cells are of the same genus. In some cases, the population of prokaryotic cells comprise bacteria of 2, 3, 4, 5, 6, 7, 8, 9, 10, or more than 10 (e.g., from 10 to 20, from 20 to 30, from 30 to 40, from 40 to 50, or more than 50), different genus and/or species. A heterogeneous population of genetically modified prokaryotic cells is also referred to as a “community” or a “prokaryotic cell community” or a “microbial community.”

The present disclosure provides a heterogeneous population of genetically modified bacteria, where the population comprises a plurality of genetically modified, which bacteria are the recipients of transposons present in a library of the present disclosure. The heterogeneous population can comprise from 10 to 10⁹ different bacteria; e.g., from 10 to 10², from 10² to 10³, from 10³ to 10⁴, from 10⁴ to 10⁵, from 10⁵ to 10⁶, from 10⁶ to 10⁷, from 10⁷ to 10⁸, or from 10⁸ to 10⁹ different bacteria, which comprise different transposons from a library of the present disclosure. In some cases, the population of bacteria are of the same genus. In some cases, the population of bacteria comprise bacteria of 2, 3, 4, 5, 6, 7, 8, 9, 10, or more than 10 (e.g., from 10 to 20, from 20 to 30, from 30 to 40, from 40 to 50, or more than 50), different genus and/or species.

### Libraries

The present disclosure provides a library of nucleic acids comprising a plurality of member conjugative nucleic acid constructs of the present disclosure. Each member conjugative nucleic acid construct comprises: a) a nucleotide sequence encoding CAST complex polypeptides; b) a nucleotide sequence encoding one or more guide RNAs, each guide RNA comprising a nucleotide sequence that hybridizes to a target nucleotide sequence in a prokaryotic cell genome; and c) a transposon, wherein the transposon is flanked by recognition sites that are cleaved by the transposase.

In some cases, nucleotide sequence encoding the CAST complex polypeptides and/or the nucleotide sequence encoding the guide RNA can be operably linked to a promoter that is functional in a prokaryotic cell. In some cases, the nucleotide sequence encoding the CAST complex polypeptides is operably linked to a first promoter; and the nucleotide sequence encoding the guide RNA is operably linked to a second promoter. In some cases, the nucleotide sequence encoding the CAST complex polypeptides and the nucleotide sequence encoding the guide RNA are operably linked to the same promoter. Suitable promoters are described above.

In some cases, each member conjugative nucleic acid construct comprises a nucleotide sequence that provides a unique nucleotide sequence barcode that identifies the member (e.g., identifies the transposon present in each member and/or identifies the guide RNA(s) encoded by each member and/or identifies the promoter, etc.).

A library of the present disclosure can comprise from 10 to 10⁹ different members; e.g., from 10 to 10², from 10² to 10³, from 10³ to 10⁴, from 10⁴ to 10⁵, from 10⁵ to 10⁶, from 10⁶ to 10⁷, from 10⁷ to 10⁸, or from 10⁸ to 10⁹ different member conjugative nucleic acid constructs of the present disclosure.

In some cases, a single member of the library can include a nucleotide sequence encoding two or more guide RNAs, each guide RNA comprising a nucleotide sequence that hybridizes to a target nucleotide sequence in a prokaryotic cell genome. For example, in some cases, a single member of the library can a nucleotide sequence encoding 2, 3, 4, or 5 (or more than 5) different guide RNAs, each targeted to a different target nucleic acid.

A library of the present disclosure can be used to target more than one gene (nucleic acid) in a prokaryotic cell. A library of the present disclosure can be used to target a subset of genes (nucleic acids) in a prokaryotic cell. A library of the present disclosure can be used to target a single gene, or more than one gene (nucleic acid), in a specific species of prokaryotic cell. A library of the present disclosure can be used to target a single gene, or more than one gene (nucleic acid), in a subset of species (e.g., 2, 3, 4, 5, 6, 7, 8, 9, or 10 species) of prokaryotic cell present in a prokaryotic cell community. A library of the present disclosure can be used to target a single gene, or more than one gene (nucleic acid), in all members of a prokaryotic cell community. In some cases, the libraries of the present disclosure include genes encoding polypeptides involved in conjugation. In some cases, the libraries of the present disclosure lack genes encoding polypeptides involved in conjugation.

### Methods of Editing the Genome of a Target Prokaryotic Cell

The present disclosure provides a method of editing the genome of a target prokaryotic cell, the method comprising introducing into the target prokaryotic cell a transposon system of the present disclosure. The present disclosure provides a method of editing the genome of a target prokaryotic cell, the method comprising introducing into the target prokaryotic cell a single conjugative construct comprising: i) a nucleotide sequence encoding polypeptides that form a CAST complex; ii) a nucleotide sequence encoding a guide RNA; and iii) a transposon, or an insertion site for a transposon, flanked by CAST complex recognition sites. In some cases, provided is a method of editing the genome of a target prokaryotic cell, the method comprising introducing into the target prokaryotic cell a single construct comprising: i) a nucleotide sequence encoding polypeptides that form a CAST complex; ii) a nucleotide sequence encoding a guide RNA; and iii) a transposon, or an insertion site for a transposon, flanked by CAST complex recognition sites.

The present disclosure provides a method of editing the genome of a target bacterium, the method comprising introducing into the target bacterium a transposon system of the present disclosure. The present disclosure provides a method of editing the genome of a target bacterium, the method comprising introducing into the target bacterium a single conjugative construct comprising: i) a nucleotide sequence encoding polypeptides that form a CAST complex; ii) a nucleotide sequence encoding a guide RNA; and iii) a transposon, or an insertion site for a transposon, flanked by CAST complex recognition sites. In some cases, the transposon system is introduced via conditions that promote introduction of nucleic acid into prokaryotic cells including by electroporation, heat shock, use of chemically induced competence or other methods known in the art.

In some cases, a method of the present disclosure for editing the genome of a target prokaryotic cell comprises contacting one or more target bacteria with one or more “donor” prokaryotic cells of the present disclosure, where the one or more “donor” prokaryotic cells comprise a transposon system of the present disclosure or a single conjugative construct of the present disclosure. The transposon system of the present disclosure or the single conjugative construct of the present disclosure is transmitted conjugatively from the one or more “donor” prokaryotic cells to the one or more target (“recipient”) prokaryotic cell. Suitable target prokaryotic cells are described above.

In some cases, an editing method of the present disclosure further comprises identifying, within the contacted target prokaryotic cells, cells that have an edited genome. In other words, in some cases, the method further comprises identifying, within the contacted target prokaryotic cells, cells that are genetically modified by the method and that, as a result of the genetic modification, have a genetically modified genome. Identification can be carried out in a number of ways, depending on the transposon transmitted to the recipient target cells. For example, where the transposon comprises a nucleotide sequence encoding a fluorescent polypeptide, recipient cells that have an edited genome can be identified by detecting fluorescence in recipient target cells.

In some cases, an editing method of the present disclosure further comprises enriching the contacted target prokaryotic cells for target cells comprising an edited genome. Enriching can be carried out by selection. For example, where the transposon comprises one or more nucleotide sequences encoding one or more polypeptides that provide for resistance to one or more antibiotics, an editing method of the present disclosure can further comprise selecting target prokaryotic cells for antibiotic resistance. The enriching step can result in an enriched population in which from 50% to more than 99% of the cells (e.g., from 50% to 60%, from 60% to 70%, from 70% to 80%, from 80% to 90%, from 90% to 95%, from 95% to 99%, or more than 99%) of the cells have a genome that has been edited as a result of the contacting step.

### Methods of Identifying Prokaryotic Cells Susceptible to Horizontal Gene Transfer and Methods of Identifying Conditions for Genetically Modifying a Prokaryotic Cell

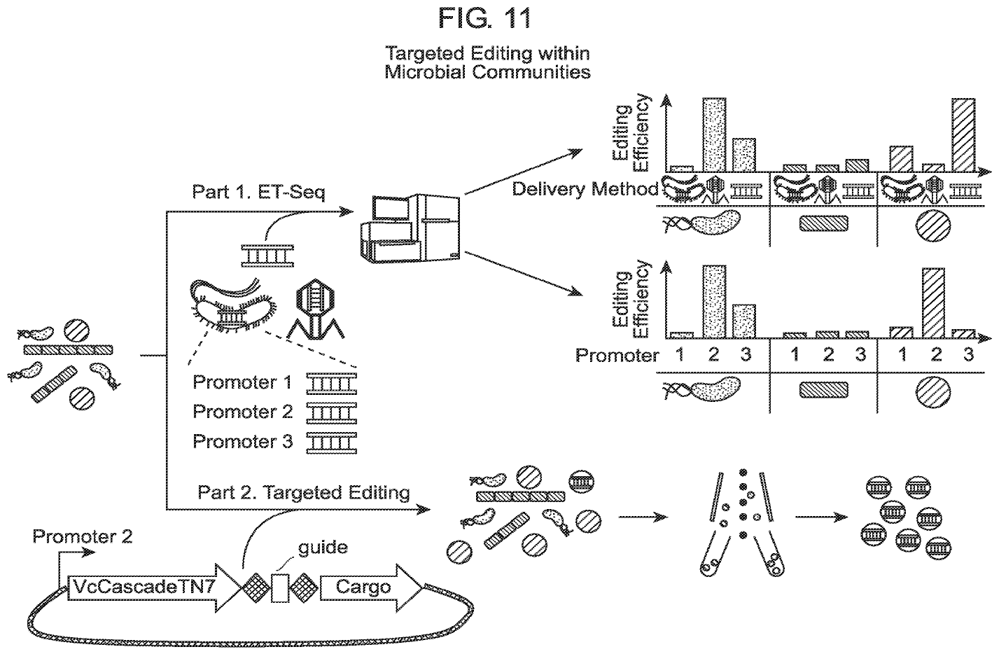

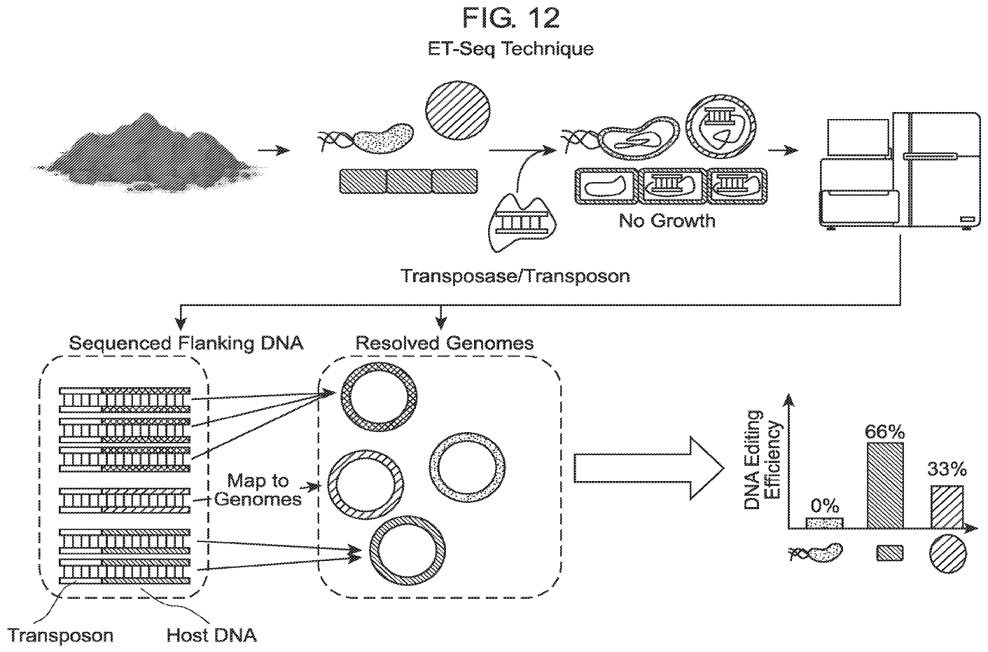

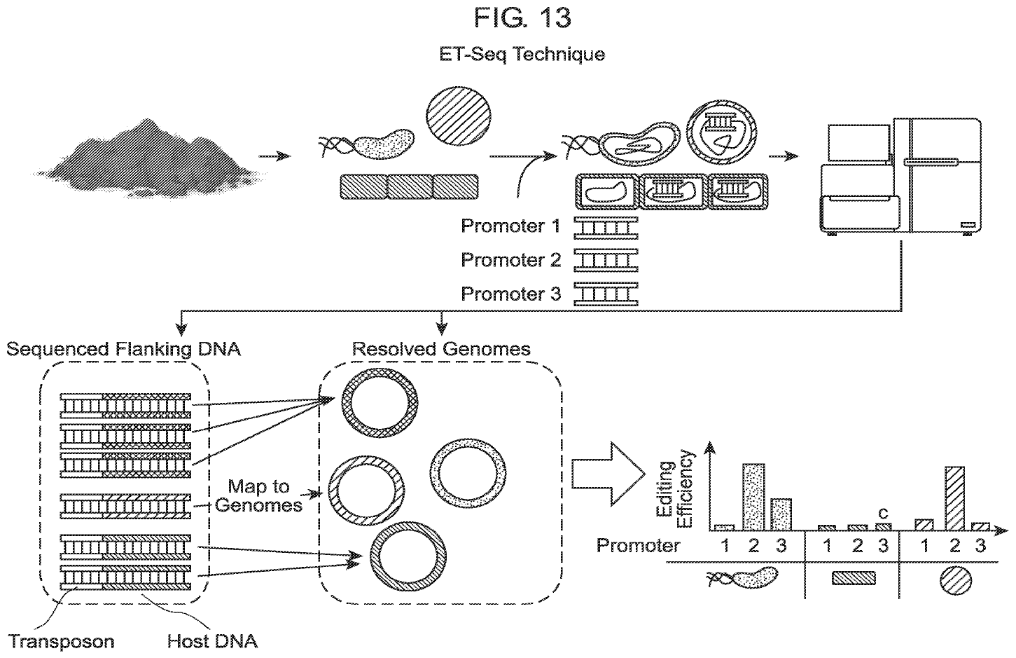

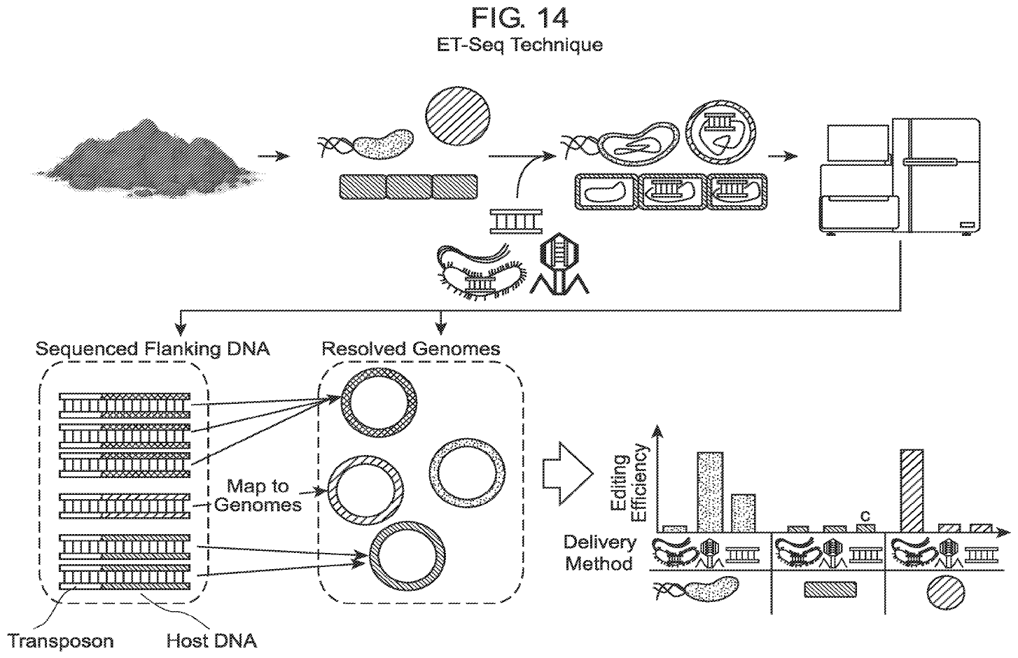

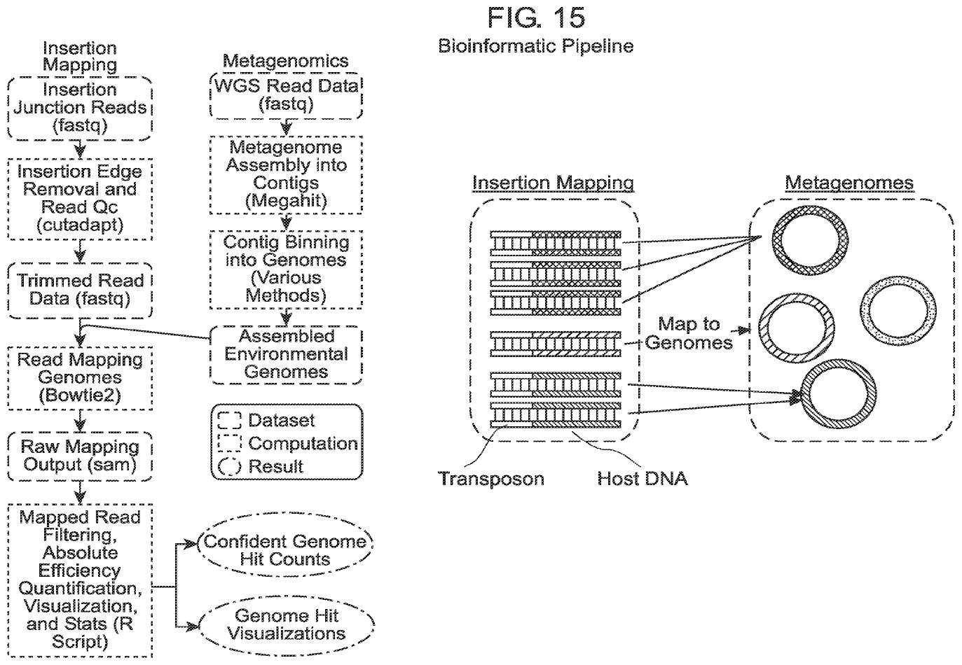

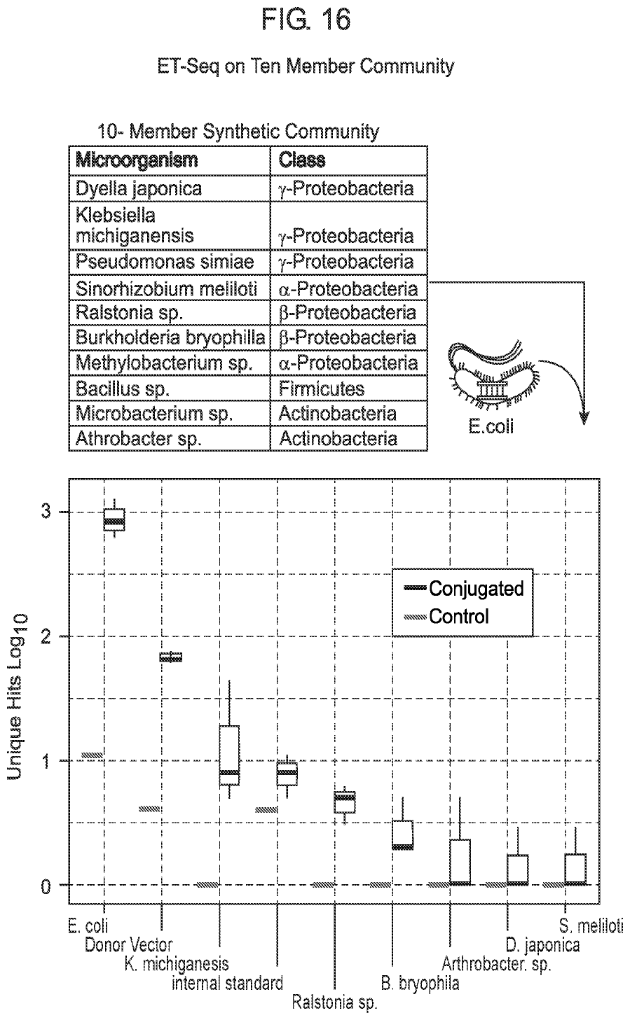

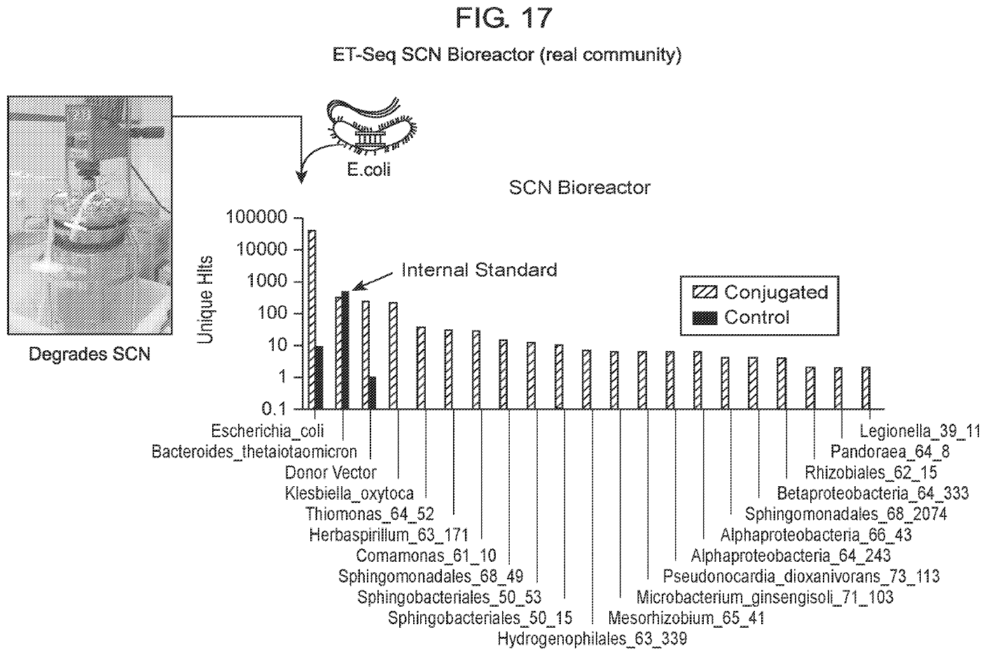

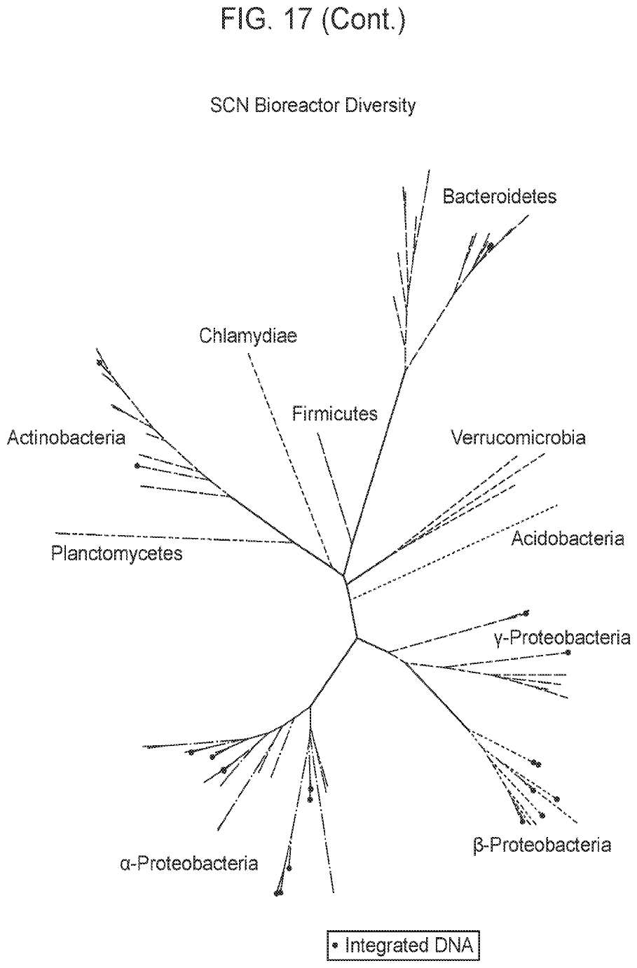

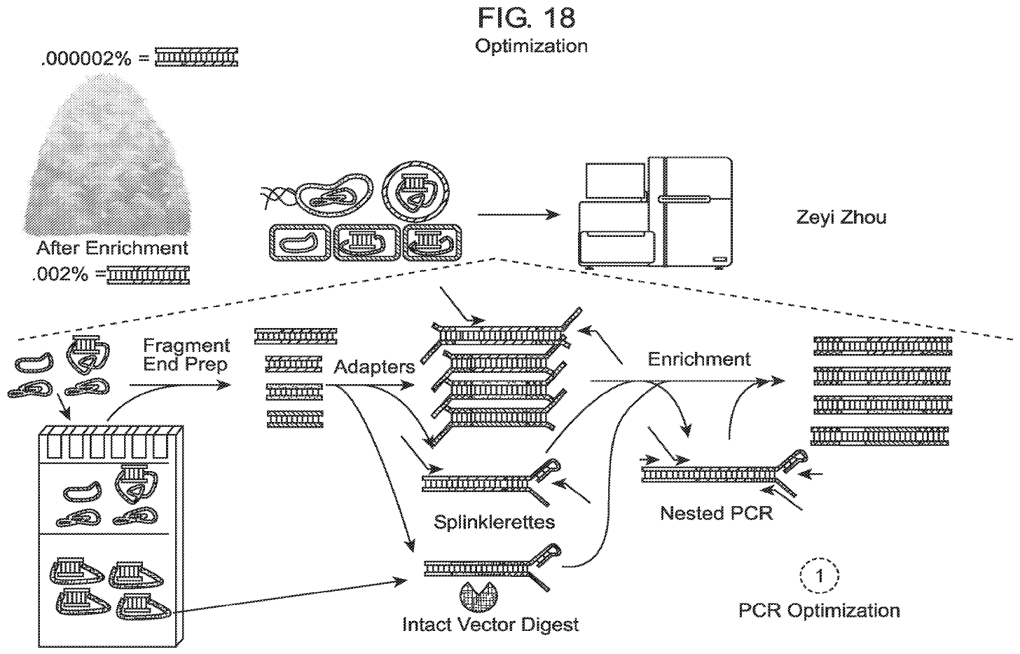

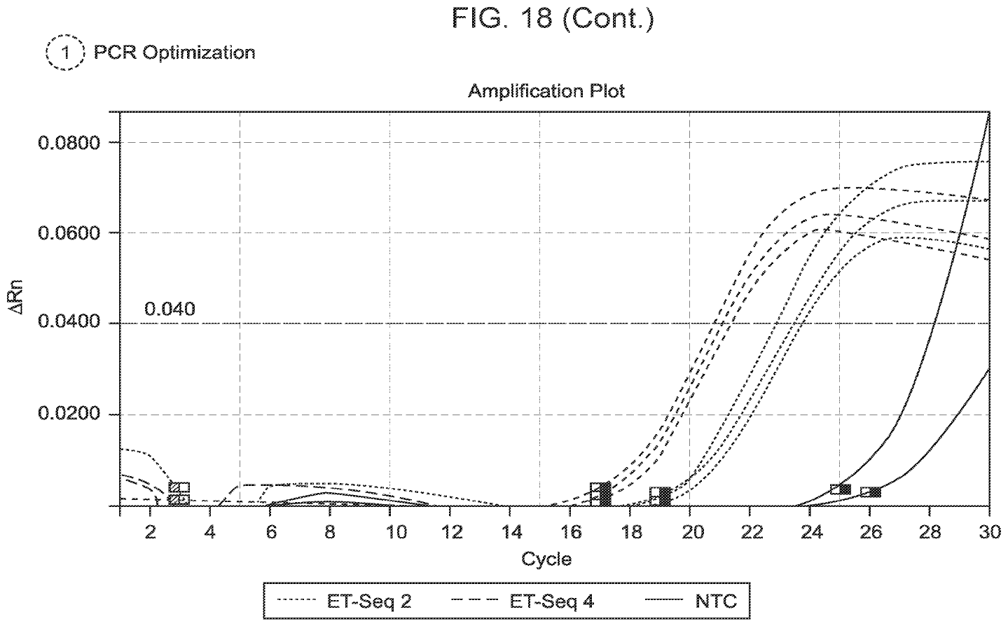

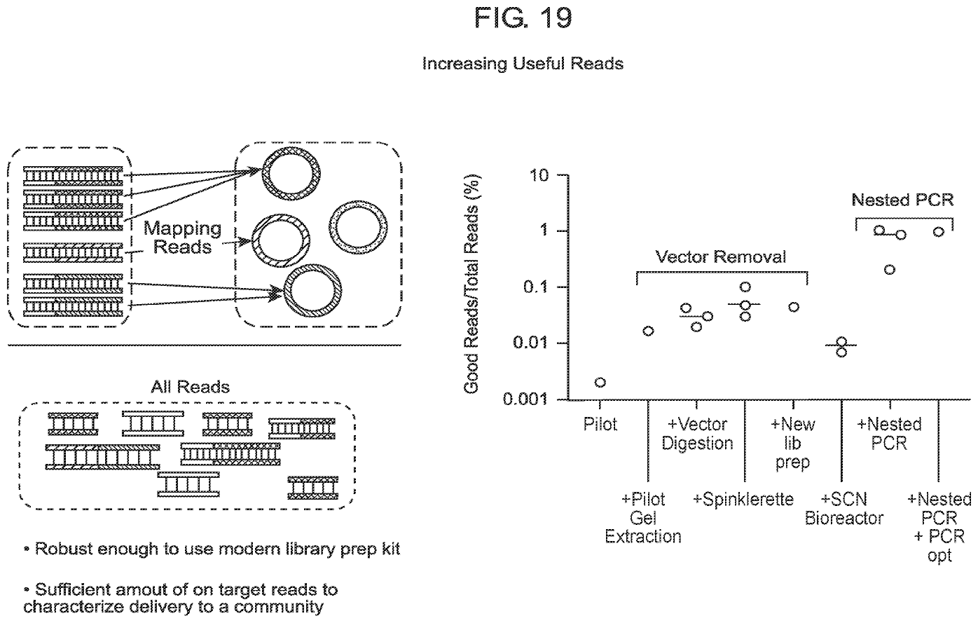

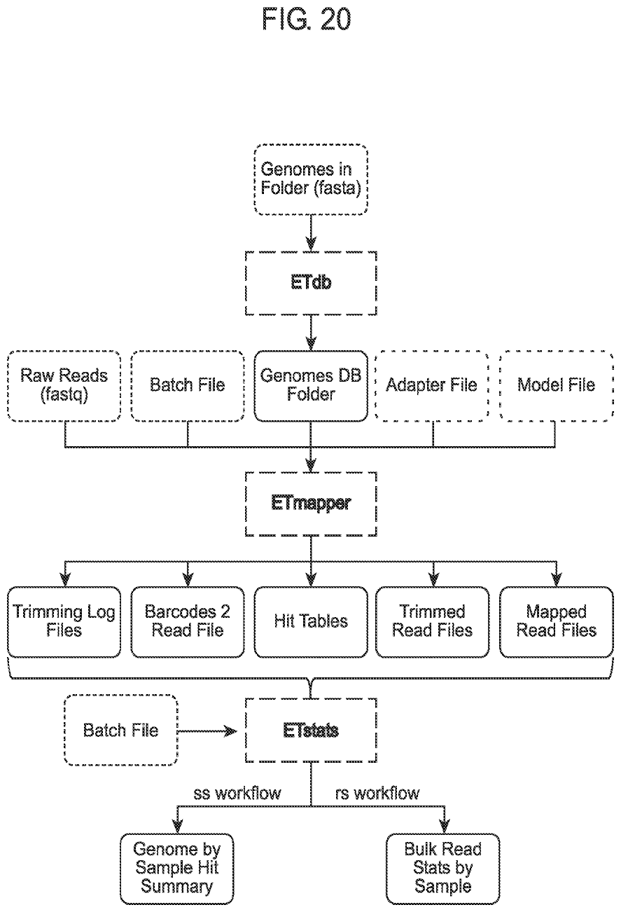

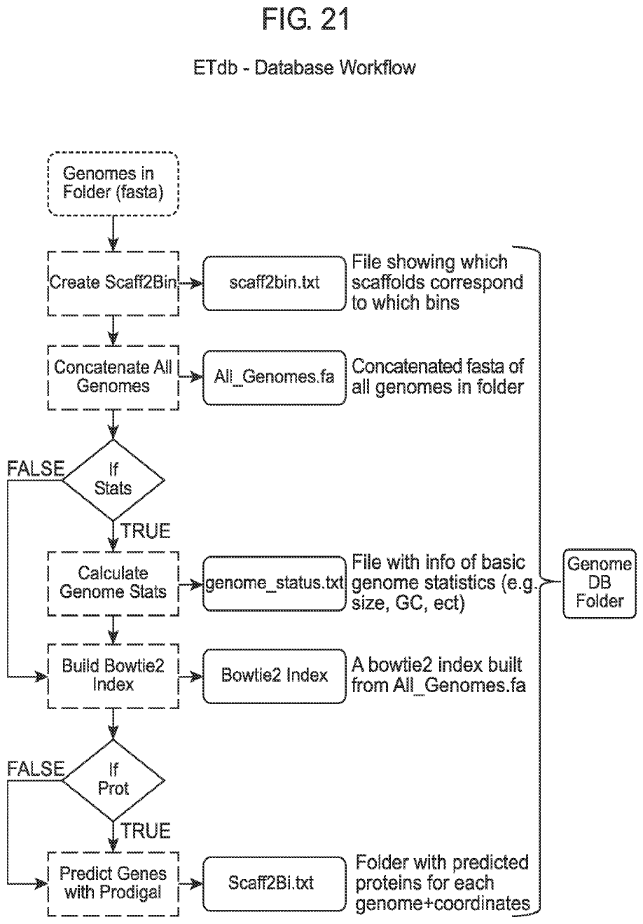

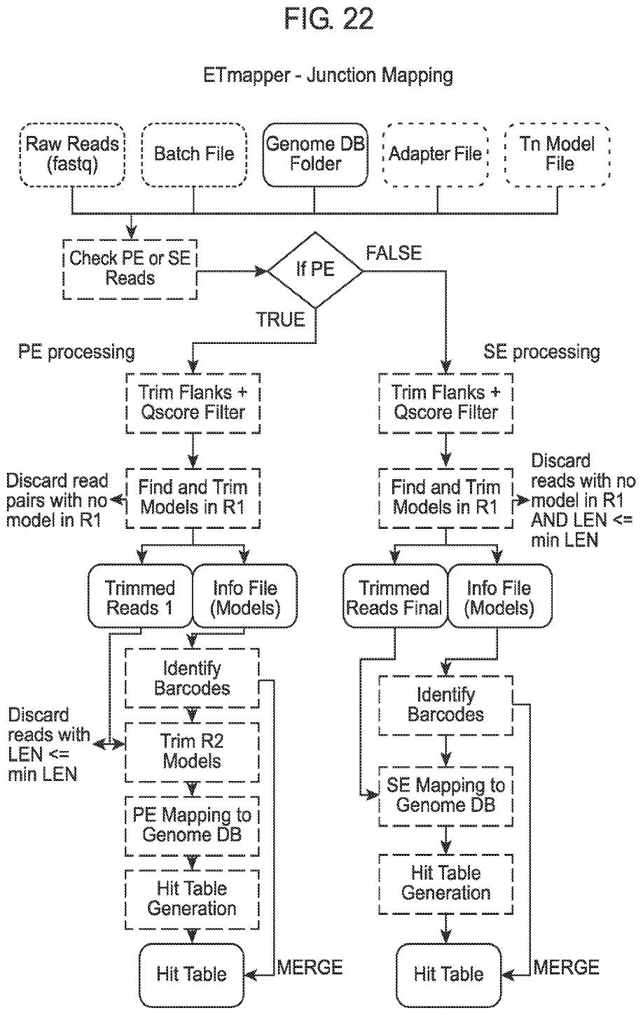

The present disclosure provides a method of identifying a prokaryotic cell that is susceptible to horizontal gene transfer (HGT); i.e., a prokaryotic cell that can function as a recipient for HGT. A prokaryotic cell that can function as a recipient for HGT comprises a genome that can be edited, e.g., using a method of the present disclosure. The present disclosure provides a method of identifying conditions for genetically modifying a prokaryotic species present in a heterogeneous population of prokaryotic cells. See, e.g., FIG. 11-22.

Methods of the present disclosure for identifying a prokaryotic cell that is susceptible to HGT, or for identifying conditions for genetically modifying a prokaryotic species present in a heterogeneous population of prokaryotic cells, comprise: a) contacting a heterogeneous population of prokaryotic cells with a library of expression vectors (also referred to as a “library of nucleic acid constructs” or “library of nucleic acids”) under conditions that promote introduction of nucleic acid into a prokaryotic cell, wherein the members of the library of expression vectors comprise a nucleotide sequence encoding a transposase and a transposon, wherein the nucleotide sequence encoding the transposase is operably linked to a promoter, wherein each member expression vector comprises a nucleotide sequence that provides a unique nucleotide sequence barcode that identifies the transposon and the promoter present in each member, wherein said contacting generates a modified heterogeneous population of prokaryotic cells comprising genetically modified prokaryotic cells comprising the transposon inserted into the genome; and b) identifying the genetically modified prokaryotic cells by sequencing the junction between the transposon and genomic DNA and/or by sequencing the nucleotide sequence barcode.

A method of the present disclosure for identifying conditions for genetically modifying a prokaryotic species present in a heterogeneous population of prokaryotic cells does not require cell sorting. A method of the present disclosure for identifying conditions for genetically modifying a prokaryotic species present in a heterogeneous population of prokaryotic cells does not require selection for acquisition of foreign nucleic acid (e.g., a heterologous expression vector not normally found in a prokaryotic cell). A method of the present disclosure for identifying conditions for genetically modifying a prokaryotic species present in a heterogeneous population of prokaryotic cells does not require that the genetically modified prokaryotic cells be isolated.

In general, the nucleotide sequence of at least a portion of the genome of the prokaryotic cells in the heterogeneous population is known or has been determined (e.g., using metagenomic sequencing).

An expression vector (a nucleic acid) in the library of expression vectors (library of nucleic acids) does not comprise a nucleotide sequence encoding CAST complex enzymes or a CRISPR/Cas effector polypeptide, or a CRISPR/Cas guide RNA. Instead, an expression vector in the library of expression vectors comprises a nucleotide sequence encoding a non-targeted transposon system (a transposon and a transposase). The present disclosure provides a library of expression vectors that comprise a nucleotide sequence encoding a transposase and a transposon, where the nucleotide sequence encoding the transposase is operably linked to a promoter, wherein each member expression vector comprises a nucleotide sequence that provides a unique nucleotide sequence barcode that identifies the transposon and the promoter present in each member. In some cases, the nucleic acids of the library of nucleic acids does not include nucleotide sequences encoding polypeptides involved in conjugation.

A transposase includes an enzyme that is capable of forming a functional complex with a transposon sequence comprising a transposon element or transposase element, and catalyzing insertion or transposition of the transposon sequence into a target nucleic acid to provide a modified nucleic acid. Insertion of the transposon sequences by the transposase can be at a random or substantially random site in the target nucleic acid. Exemplary transposases that may be used include, but are not limited to, transposases from the transposon systems Tn1, Tn2, Tn3, Tn5, Tn7, Tn9, Tn10, Tn903, Tn1000/Gamma-delta, Minos, Sleeping beauty, piggyBac, Tol2, Mos1, Himar1, Hermes, Tol2, Minos, P-element, Tc1/mariner, Tc3, or biologically active variants thereof.

Exemplary transposases include, but are not limited to Mu, Tn10, Tn5, and hyperactive Tn5 (See, e.g., Goryshin and Reznikoff (1998) J. Biol. Chem. 273:7367). See, e.g., U.S. 2010/0120098. Other suitable transposases and transposon elements include a hyperactive Tn5 transposase and a Tn5-type transposase element (Goryshin and Reznikoff (1998) supra), MuA transposase and a Mu transposase element comprising R1 and R2 end sequences (Mizuuchi (1983) Cell 35:785; and Savilahti et al. (1995) EMBO J. 14:4893). Examples of transposase elements that form a complex with a hyperactive Tn5 transposase (e.g., EZ-Tn5™ Transposase, Epicentre Biotechnologies, Madison, Wis.) are set forth in WO 2012/061832; U.S. 2012/0208724, U.S. 2012/0208705 and WO 2014018423. Other suitable transposases and transposon sequences include Staphylococcus aureus Tn552 (Colegio et al. (2001) J. Bacteriol. 183: 2384-8; Kirby et al. (2002) Mol. Microbiol. 43:173-86); Ty1 (Devine and Boeke (1994) Nucleic Acids Res. 22:3765-72; and WO 95/23875); Transposon Tn7 (Craig (1996) Science 271:1512; Craig (1996) Curr Top Microbiol Immunol. 204:27-48); Tn/O and IS10 (Kleckner et al. (1996) Curr Top Microbiol Immunol. 204:49-82); Mariner transposase (Lampe et al. (1996) EMBO J. 15:5470-9); Tel (Plasterk (1996) Curr. Topics Microbiol. Immunol. 204:125-43); P Element (Gloor (2004) Methods Mol. Biol. 260:97-114); Tn3 (Ichikawa and Ohtsubo (1990) J Biol. Chem. 265:18829-32); and the like. Other suitable examples include ISS, Tn10, Tn903, IS911, and engineered versions of transposase family enzymes; and MuA transposases. Variants of Tn5 transposases, such as having amino acid substitutions, insertions, deletions, and/or fusions with other proteins or peptides are also suitable for use.

In some cases, instead of using an expression vector comprising a nucleotide sequence encoding a transposase, a method of the present disclosure comprises contacting a heterogeneous population of prokaryotic cells with a linear nucleic acid (e.g., a library of linear nucleic acids) complexed with a transposase; in other words, the transposase is pre-bound to the transposon. The linear nucleic acids can be polymerase chain reaction (PCR) products.

In some cases, a transposon sequence comprises a double-stranded nucleic acid. A transposon element includes a nucleic acid comprising a nucleotide sequences that form a complex with a transposase or integrase enzyme. In some cases, a transposon element is capable of forming a functional complex with the transposase in a transposition reaction. Examples of transposon elements are provided herein, and include the 19-bp outer end (“OE”) transposon end, inner end (“IE”) transposon end, or “mosaic end” (“ME”) transposon end recognized by, for example, a wild-type or mutant Tn5 transposase, or the R1 and R2 transposon end (See e.g., US 2010/0120098). Transposon elements can comprise any nucleic acid suitable for forming a functional complex with the transposase or integrase enzyme in an in vitro transposition reaction. For example, the transposon end can comprise DNA, RNA, modified bases, non-natural bases, modified backbone, and can comprise nicks in one or both strands.

In some cases, a transposon can include one or more additional elements (additional nucleotide sequences). The additional sequences can include a primer binding site, such as a promoter, a sequencing primer site and an amplification primer site, a nucleotide sequence barcode, and the like. For example, in some cases, each member expression vector of the library of expression vectors comprises a unique nucleotide sequence barcode that identifies the member (e.g., identifies the transposon and/or the promoter).

As noted above, a subject method for identifying conditions for genetically modifying a prokaryotic species present in a heterogeneous population of prokaryotic cells comprises contacting a heterogeneous population of prokaryotic cells with a library of expression vectors under conditions that promote introduction of nucleic acid into a prokaryotic cell. In some cases, a subject method comprises subjecting the heterogeneous population of prokaryotic cells to conditions for conjugation, transformation, or transduction, where such conditions permit conjugation, transformation, or transduction of a prokaryotic cell known to be susceptible to nucleic acid transfer via conjugation, transformation, or transduction. In some cases, the conditions comprise electroporation. For example, in some cases, a heterogeneous population of prokaryotic cells is electroporated in a liquid medium comprising a library of expression vectors. In some cases, the conditions comprise chemically induced competence (e.g., calcium chloride; rubidium chloride; etc.).

In some cases, genetically modified prokaryotic cells are identified by sequencing the junction between the transposon and genomic DNA and/or by sequencing the nucleotide sequence barcode. In some cases, a method of the present disclosure comprises: a) contacting a heterogeneous population of prokaryotic cells with a library of expression vectors under conditions that promote introduction of nucleic acid into a prokaryotic cell, wherein the members of the library of expression vectors comprise a nucleotide sequence encoding a transposase and a transposon, wherein the nucleotide sequence encoding the transposase is operably linked to a promoter, wherein each member expression vector comprises a nucleotide sequence that provides a unique nucleotide sequence barcode that identifies the transposon and the promoter present in each member, wherein said contacting generates a modified heterogeneous population of prokaryotic cells comprising genetically modified prokaryotic cells comprising the transposon inserted into the genome; b) fragmenting DNA obtained from the modified heterogeneous population of prokaryotic cells; c) ligating adaptor DNA fragments to the fragmented DNA; and d) amplifying the junction between the transposon and genomic DNA by polymerase chain reaction (PCR), using a forward PCR primer that hybridizes to a nucleotide sequence in the transposon and a reverse PCR primer that hybridizes to a nucleotide sequence in the adaptor DNA. For example, DNA can be obtained from the modified heterogeneous population of prokaryotic cells by standard methods (e.g., detergent lysis; physical disruption (e.g., bead beading); ultrasonic lysis; and the like). The DNA obtained can be fragmented, and adaptor DNA fragments ligated to the fragmented DNA. Multiple rounds of PCR amplification can be carried out. In some cases, both the bar code and the junction are sequenced. The nucleotide sequence of the junction provides a partial nucleotide sequence of the genome. The partial nucleotide sequence of the genome is compared with known nucleotide sequences of genomes of prokaryotic cells; and provides for identification of prokaryotic cells within the heterogeneous population that have been recipients of a member of the library of expression vectors. Sequencing the barcode provides the identity of the individual member of the library of expression vectors, including the promoter present in each member of the library; as such the method also provides for identification of which promoters, within the library of expression vectors, that is functional in a particular species of prokaryotic cell within the community of prokaryotic cells.

Suitable prokaryotic cells include bacteria and archaea, as described above.

Suitable heterogeneous populations of prokaryotic cells can be found in a natural environment such as the gastrointestinal tract of a mammal (e.g., a human); the microbiome of a human; the microbiome of a non-human animal soil; hot springs; oceans; marshland; swamps; etc. Suitable heterogeneous populations of prokaryotic cells include prokaryotic cells found in wastewater, agricultural runoff, and the like. Suitable heterogeneous populations of prokaryotic cells include prokaryotic cells involved in food processing (e.g., fermentations to produce beverages or food that rely on a mixed community of cells such as with kimchi, soy sauce, or kombucha). Suitable heterogeneous populations of prokaryotic cells present in the rhizosphere. Suitable heterogeneous populations of prokaryotic cells present on the plant surface (the plant microbiome). Suitable heterogeneous populations of prokaryotic cells found in industrial processes relying on communities of microorgansisms such as industrial wastewater treatment or bioreactors used for bioremediation of wastes (i.e. thiocyanate (SCN) degradation reactors used for gold mining runoff).

A heterogeneous population of prokaryotic cells can include from 5 to 5000, or more than 5000, different species. For example, a heterogeneous population of prokaryotic cells can include from 5 to 25, from 25 to 50, from 50 to 100, from 100 to 250, from 250 to 500, from 500 to 1000, from 1000 to 2000, from 2000 to 3000, from 3000 to 4000, from 4000 to 5000, or more than 5000, different species.

A method of the present disclosure for identifying conditions for genetically modifying a prokaryotic species present in a heterogeneous population of prokaryotic cells can provide for identification of one or more of: a) conditions (e.g., electroporation; chemically-induced competence; etc.) for introducing a heterologous nucleic acid into a prokaryotic species; b) promoters that will function in a prokaryotic species; and c) efficiency of genome editing of a prokaryotic species. A method of the present disclosure is also referred to as “Environmental Transformation Sequencing” (“ET-Seq”) and comprises delivery of a non-targeted transposon (a library of expression vectors (“delivery vectors”) as described above) to a prokaryotic cell community (a heterogeneous population prokaryotic cells) and sequencing to determine which prokaryotic cells in the community are editable. Delivery of the library of expression vectors (“delivery vectors”) is repeated with multiple delivery techniques to determine which delivery techniques work (provide for genetic modification) for which members of the community. The delivery vectors are multiplexed with multiple promoters allowing the determination of which promoters function in which members of the community. The information garnered from ET-Seq can be used to guide a targeted transposon into a particular locus within a single community member (targeted editing).

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-27 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A transposon system comprising:

a) a nucleotide sequence encoding polypeptides that form a CRISPR-associated transposase (CAST) complex;

b) a nucleotide sequence encoding a guide RNA comprising a nucleotide sequence that hybridizes to a target nucleotide sequence in a prokaryotic cell genome; and

c) a transposon, or an insertion site for a transposon, wherein the transposon or the transposon insertion site is flanked by recognition sites that are recognized by the CAST complex,

wherein (a) and (b) are present on the same nucleic acid construct.

Aspect 2. The system of aspect 1, wherein (a), (b), and (c) are all present on the same nucleic acid construct.

Aspect 3. The system of aspect 1, wherein the nucleic acid construct is a conjugative nucleic acid construct.

Aspect 4. The system of aspect 2, wherein the nucleic acid construct is a conjugative nucleic acid construct.

Aspect 5. The system of aspect 1, wherein the CAST complex comprises:

a) a Cas12k polypeptide, a tnsC polypeptide, a tnsB polypeptide, and a tniQ polypeptide; or

b) a Cas6 polypeptide, a Cas7 polypeptide, a Cas8 polypeptide, a tnsA polypeptide, a tnsB polypeptide, a tnsC polypeptide, and a tniQ polypeptide.

Aspect 6. The system of aspect 5, wherein:

a) the Cas12k polypeptide comprises an amino acid sequence having at least 50% amino acid sequence identity to the amino acid sequence depicted in any one of FIG. 4A and FIG. 6F-6J;

b) the tnsC polypeptide comprises an amino acid sequence having at least 50% amino acid sequence identity to the amino acid sequence depicted in any one of FIG. 4C and FIG. 6L-6N;

c) the tnsB polypeptide comprises an amino acid sequence having at least 50% amino acid sequence identity to the amino acid sequence depicted in any one of FIG. 4B and FIG. 6A-6E; and

d) the tniQ polypeptide comprises an amino acid sequence having at least 50% amino acid sequence identity to the amino acid sequence depicted in any one of FIG. 4D and FIG. 6O-6R.

Aspect 7. The system of aspect 5, wherein:

a) the Cas6 polypeptide comprises an amino acid sequence having at least 50% amino acid sequence identity to the amino acid sequence depicted in any one of FIG. 5G and FIG. 7M-7O;

b) the Cas7 polypeptide comprises an amino acid sequence having at least 50% amino acid sequence identity to the amino acid sequence depicted in any one of FIG. 5F and FIG. 7P-7R;

c) the Cas8 polypeptide comprises an amino acid sequence having at least 50% amino acid sequence identity to the amino acid sequence depicted in any one of FIG. 5E and FIG. 7S-7U;

d) the tnsA polypeptide comprises an amino acid sequence having at least 50% amino acid sequence identity to the amino acid sequence depicted in any one of FIG. 5A and FIG. 7A-7C;

e) the tnsB polypeptide comprises an amino acid sequence having at least 50% amino acid sequence identity to the amino acid sequence depicted in any one of FIG. 5B and FIG. 7D-7F;

f) the tnsC polypeptide comprises an amino acid sequence having at least 50% amino acid sequence identity to the amino acid sequence depicted in any one of FIG. 5C and FIG. 7G-7I; and

g) the tniQ polypeptide comprises an amino acid sequence having at least 50% amino acid sequence identity to the amino acid sequence depicted in any one of FIG. 5D and FIG. 7J-7L.

Aspect 8. The system of any one of aspects 1-7, wherein the transposon has a size of up to 100 kb.

Aspect 9. The system of any one of aspects 1-8, wherein the construct comprises a promoter operably linked to the nucleotide sequence encoding the CAST complex polypeptides and to the nucleotide sequence encoding the guide RNA, wherein the promoter is functional in a prokaryotic cell.

Aspect 10. The system of any one of aspects 1-9, wherein the construct comprises a selectable marker.

Aspect 11. The system of any one of aspects 1-9, wherein the construct does not comprise a selectable marker.

Aspect 12. The system of any one of aspects 1-11, wherein the transposon comprises one or more nucleotide sequences encoding one or more polypeptides that confer antibiotic resistance on a bacterium.

Aspect 13. The system of any one of aspects 1-11, wherein the transposon comprises one or more nucleotide sequences encoding one or more enzymes in a biosynthetic pathway.

Aspect 14. The system of any one of aspects 1-11, wherein the transposon comprises one or more nucleotide sequences encoding a polypeptide that inhibits viability and/or growth of a prokaryotic cell.

Aspect 15. The system of any one of aspects 1-11, wherein the transposon comprises one or more nucleotide sequences encoding one or more enzymes in a carbon utilization pathway.

Aspect 16. The system of aspect 15, wherein the carbon utilization pathway is a polysaccharide utilization pathway.

Aspect 17. The system of any one of aspects 1-16, wherein the transposon comprises one or more nucleotide sequences encoding one or more detectable markers.

Aspect 18. The system of aspect 17, wherein the detectable marker is a fluorescent polypeptide.

Aspect 19. A prokaryotic cell comprising the system of any one of aspects 1-18.

Aspect 20. A library of nucleic acids comprising a plurality of member conjugative nucleic acid constructs, wherein each member conjugative nucleic acid construct comprises:

a) a nucleotide sequence encoding CRISPR-associated transposase (CAST) complex polypeptides;

b) a nucleotide sequence encoding a guide RNA comprising a nucleotide sequence that hybridizes to a target nucleotide sequence in a prokaryotic cell genome;

c) a transposon, wherein the transposon is flanked by recognition sites that are cleaved by the transposase.

Aspect 21. The library of aspect 20, wherein each member conjugative nucleic acid construct comprises a nucleotide sequence that provides a unique nucleotide sequence barcode that identifies the member.

Aspect 22. A library of prokaryotic cells comprising the library of aspect 20 or aspect 21.

Aspect 23. A method of editing the genome of a target prokaryotic cell, the method comprising introducing into the target bacterium the transposon system of any one of aspects 1-18.

Aspect 24. The method of aspect 23, wherein said introducing comprises contacting one or more target prokaryotic cells with one or more prokaryotic cells according to aspect 19, and wherein the construct is transmitted conjugatively from said one or more prokaryotic cells to the one or more target prokaryotic cell.

Aspect 25. The method of aspect 23 or aspect 24, wherein the one or more target prokaryotic cells are: a) one or more prokaryotic cells present in or enriched from a natural environment; or b) one or more prokaryotic cells present in a synthetic community of prokaryotic cells.

Aspect 26. The method of aspect 25, wherein the one or more one target prokaryotic cells are one or more gut bacteria.

Aspect 27. The method of aspect 25, wherein the natural environment comprises soil.

Aspect 28. The method of any one of aspects 23-25, wherein the one or more target prokaryotic cells are refractory to genetic modification by electroporation and/or heat shock.

Aspect 29. The method of any one of aspects 23-28, wherein the target prokaryotic cells are a heterogeneous population of prokaryotic cells.

Aspect 30. The method of any one of aspects 23-29, wherein said introducing comprises contacting a population of target prokaryotic cells with said one or more prokaryotic cells, and wherein the method comprises, after said introducing,

identifying target cells, within the contacted population of target prokaryotic cells, that have an edited genome and/or

enriching the contacted population of target prokaryotic cells for target cells having an edited genome.

Aspect 31. The method of aspect 30, wherein said identifying comprises high throughput nucleic acid sequencing.

Aspect 32. The method of aspect 31, wherein the transposon comprises a distinguishable marker and said enriching is based on a phenotype associated with the presence or absence of the distinguishable marker.

Aspect 33. The method of aspect 32, wherein the distinguishable marker is a screenable marker.

Aspect 34. The method of aspect 33, wherein the screenable marker is a fluorescent protein encoded by the transposon.

Aspect 35. The method of aspect 33, wherein the screenable marker is an epitope encoded by the transposon.

Aspect 36. The method of aspect 33, wherein the screenable marker is a fluorescent aptamer encoded by the transposon.

Aspect 37. A library of nucleic acids comprising a plurality of member nucleic acids, wherein each member nucleic acid comprises:

a) a nucleotide sequence encoding a transposon, wherein the transposon is flanked by recognition sites that are cleaved by a transposase; and

b) a nucleotide sequence that provides a unique nucleotide sequence barcode that identifies the member.

Aspect 38. The library of aspect 37, wherein each member nucleic acid comprises a nucleotide sequence encoding the transposase.

Aspect 39. The library of aspect 37, comprising a transposase bound to a member nucleic acid.

Aspect 40. The library of any one of aspects 37-39, wherein each member nucleic acid comprises a promoter operably linked to the transposon.

Aspect 41. A method of identifying conditions for genetically modifying a prokaryotic species present in a heterogeneous population of prokaryotic cells, the method comprising:

a) contacting the heterogeneous population of prokaryotic cells with a library of nucleic acids according to any of aspects 37-40 under conditions that promote introduction of nucleic acid into a prokaryotic cell, wherein said contacting generates a modified heterogeneous population of prokaryotic cells comprising genetically modified prokaryotic cells comprising the transposon inserted into the genome; and

b) identifying the species of genetically modified prokaryotic cells by sequencing the junction between the transposon and genomic DNA and/or by sequencing the nucleotide sequence barcode.

Aspect 42. The method of aspect 41, wherein the conditions that promote introduction of nucleic acid into a prokaryotic cell comprise conjugation, transformation, or transduction.

Aspect 43. The method of aspect 41, wherein the conditions that promote introduction of nucleic acid into a prokaryotic cell comprise electroporation or chemically induced competence.

Aspect 44. The method of any one of aspects 41-43, wherein the transposon and transposase are from a Tn5 system or a Mariner system.

Aspect 45. The method of any one of aspects 37-40, comprising, after step (a), amplifying the junction between the transposon and genomic DNA.

Aspect 46. The method of aspect 45, comprising:

a) fragmenting DNA obtained from the modified heterogeneous population of prokaryotic cells;

b) ligating adaptor DNA fragments to the fragmented DNA; and

c) amplifying the junction between the transposon and genomic DNA by polymerase chain reaction (PCR), using a forward PCR primer that hybridizes to a nucleotide sequence in the transposon and a reverse PCR primer that hybridizes to a nucleotide sequence in the adaptor DNA.

Aspect 47. The method of any one of aspects 41-46, wherein the heterogeneous population of prokaryotic cells comprises at least 5 different species of prokaryotic cells.

Aspect 48. The method of any one of aspects 41-46, wherein the heterogeneous population of prokaryotic cells comprises from 5 to 50 or from 50 to 500 different species of prokaryotic cells.

Aspect 49. The method of any one of aspects 41-48, wherein the heterogeneous population of prokaryotic cells is obtained from a soil sample.

Aspect 50. The method of any one of aspects 41-48, wherein the heterogeneous population of prokaryotic cells are from the intestinal tract of a mammal

Aspect 51. The method of any one of aspects 41-48, wherein the heterogeneous population of prokaryotic cells are present in bioremediation, food, food processing, a bioreactor, an SCN bioreactor, or waste processing.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

A generalizable approach to edit specific members and loci within a microbial community is described. First, genetically accessible bacteria within a community were identified using an Environmental Transformation Sequencing (ET-Seq) approach, in which non-targeted transposons were delivered to a community and had their insertion sites mapped and quantified. ET-Seq was repeated with multiple delivery strategies on a nine-member synthetic consortium and ˜200-member bioremediation community. Insertions in 10 species not previously isolated were achieved. Natural competence that is dependent on the presence of the community was identified. Second, a DNA-editing All-in-one RNA-guided CRISPR-Cas Transposase (DART) system was developed and used for targeted insertion of DNA into organisms identified as tractable by ET-Seq, enabling organism and locus specific manipulation within the community context. These results establish a general strategy for targeted genetic manipulation of microbial communities relevant for both basic and applied biology in human, environmental, and industrial microbiomes.

### Methods

### Data Reporting

No statistical methods were used to predetermine sample size. The experiments were not randomized, and investigators were not blinded to allocation during experiments and outcome assessment.

### Plasmid Construction and Barcoding

For ET-Seq measurement of genetic tractability in community members, DNA encoding a non-targeted mariner transposon was delivered. The mariner transposon integrates into “TA” sequences in recipient cells. For delivery of the mariner transposon, use was made of the previously created pHLL250 vector, which contains an RP4 origin of transfer (oriT), AmpR, conditional (pie-dependent) R6K origin, and an AseI restriction site to facilitate depletion of vector from DNA samples in ET-Seq library preparations. Unique to each transposon on this vector is a random 20 bp barcode sequence to aid in the discrimination of unique insertion events from duplications of the same insertion due to cell division or PCR.

DART vectors were designed to encode all components required for delivery and editing. VcCasTn genes, crRNA, and Tn were synthesized as gBlocks (IDT). pHelper_ShCAST_sgRNA (Addgene plasmid #127921; http://n2t.net/addgene:127921; RRID:Addgene_127921) was used to clone ShCasTn genes and sgRNA. pHelper_ShCAST_sgRNA (Addgene plasmid #127921; http://n2t.net/addgene:127921; RRID:Addgene_127921) was used to clone the ShCasTn transposon. tns genes, cas genes, and crRNA/sgRNA were consolidated into a single operon (with various promoters and transcriptional configurations) on the same vector as the cognate transposon. The left end of the cognate Tn was encoded downstream of the crRNA/sgRNA, followed by Tn cargo, barcode, and Tn right end. DART Tn LE and RE were designed to include the minimal sequence that both included all putative TnsB binding sites and was previously shown to be functional. Specifically, VcDART LE (108 bp) and RE (71 bp) each encompass three 20 bp putative TnsB binding sites, spanning from the edge of the 8 bp terminal ends to the edge of the third putative TnsB binding site. ShDART LE (113 bp) spans the boundaries of the long terminal repeat and both additional putative TnsB binding sites, while the RE (211 bp) encompasses the long terminal repeat and all four additional putative TnsB binding sites.

Vectors were cloned using BbsI (NEB) Golden Gate assembly of part plasmids, each encoding different regions of the final plasmid. Of note, the backbone encodes RP4 oriT, AmpR, conditional R6K origin, and an AsiSI+SbfI double digestion site for vector depletion during ET-Seq library preparations. A 2×BsaI spacer placeholder enabled spacer cloning with BsaI (NEB) Golden Gate. A 2×BsmBI barcode placeholder was encoded immediately inside the Tn right end and was used for barcoding as described below. Part plasmids were propagated in E. coli Mach1-T1R (QB3 Macro Lab). Golden Gate reactions for all-in-one vector assembly were purified with DNA Clean & Concentrator-5 (Zymo Research) and electroporated into E. coli EC100D-pir+(Lucigen).

DART vectors were barcoded by BsmBI (NEB) Golden Gate insertion of random barcode PCR product into the 2×BsmBI barcode placeholder using a previously reported method with slight modifications. A 56-nt ssDNA oligonucleotide encoding a central tract of 20 degenerate nucleotides (oBFC1397) was amplified with BsmBI-encoding primers oBFC1398 and oBFC1399 using Q5 High-Fidelity 2× Master Mix (NEB) in a six-cycle PCR (98° C. for 1 min; six cycles of 98° C. for 10 s, 58° C. for 30 s, and 72° C. for 60 s; and 72° C. for 5 min). Barcoding Golden Gate reactions were purified with DNA Clean & Concentrator-5. To remove residual non-barcoded vector, reactions were digested with 15 U BsmBI at 55° C. for at least 4 hr, heat inactivated at 80° C. for 20 min, treated with 10 U Plasmid-Safe ATP-Dependent DNase (Lucigen) exonuclease at 37° C. for 1 hr, heat inactivated at 70° C. for 30 min, and purified with DNA Clean & Concentrator-5.

Randomly barcoded conjugative vectors were electroporated into E. coli EC100D-pir+, followed 1 hr recovery in 1 mL pre-warmed SOC (NEB) at 37° C. 250 rpm, serial dilution and spot plating on LB agar plus 100 ug mL-1 carbenicillin to estimate library diversity, and plating the full transformation across 5 LB agar plates containing carbenicillin (and other appropriate antibiotics when Tn cargo contained other resistance cassettes). To prepare barcoded conjugative vector plasmid stock, all 5 agar plates were scraped into a single pool and midiprepped (Zymo Research). All conjugations were performed using the diaminopimelic acid (DAP) auxotrophic RP4 conjugal donor E. coli strain WM3064. Donor strains were prepared by electroporation with 200 ng barcoded vectors, followed by recovery in SOC plus DAP at 37° C. and 250 rpm and inoculation of the entire recovery culture into 15 mL LB containing DAP and carbenicillin in 50 mL conical tubes, followed by overnight cultivation at 37° C. and 250 rpm. Donor serial dilutions were spot plated on LB agar plus carbenicillin to estimate final barcode diversity.

### Guide RNA Design

In all experiments, VcCasTn gRNAs used 32 nt spacers and a 5′-CC Type IF PAM, while ShCasTn gRNAs used 23 nt spacers and a 5′-GTT Cas12k PAM. All gRNAs were designed to bind in the first ½ of the target CDS to ensure functional knockout. Off-target potential was assessed using BLASTn (-dust no -word_size 4) of spacers against a local BLAST database created from all genomes present in an experiment, and spacers were discarded if off-target hits with E-value <15 were identified. gRNAs with less seed region complementarity to off-targets were prioritized. Non-targeting gRNAs were designed by scrambling the spacer until no significant matches were found.

### Delivery Methods

For natural transformation and electroporation, a culture of the community or isolate to be transformed was subcultured at OD₆₀₀=0.2 and grown to OD₆₀₀=0.5. In the case of the thiocyanate bioreactor in the absence of accurate OD measurements the culture was outgrown for two hours. To transform, 200 ng of vector harboring the mariner transposon (pHLL250) for non-targeted editing, or water for the negative control were added to 4 mL of OD₆₀₀=0.5 outgrowth. Cultures were incubated for 3 hours shaking at 250 rpm at temperature appropriate for the isolate or community before being moved to the appropriate downstream analysis.

For electroporation, 20 mL of the community or isolate at OD₆₀₀=0.5 was put on ice, centrifuged at 4,000 g at 4° C. for 10 minutes, and washed four times with 10 mL sterile ice-cold Milli-Q H₂O. After a final centrifugation the pellet was resuspended in 100 μL of 2 ng/μL vector (pHLL250 or vcCasTn), or 100 μL of water as a negative control. This solution was then pipetted into a 0.2 cm gap ice-cold cuvette and electroporated at 3 kV, 200Ω, and 25 μF. The cells were immediately recovered into 10 mL of the community's or isolate's preferred medium and incubated shaking for 3 hours before being moved to the appropriate downstream analysis.

E. coli strain WM3064 containing the mariner transposon (pHLL250) for non-targeted editing, or the VcDART for targeted editing was cultured overnight in LB supplemented with carbenicillin (100 μg/mL) and DAP (60 μg/mL) at 37° C. Before conjugation the donor strain was washed twice in LB (centrifugation at 4,000 g for 10 minutes) to remove antibiotics. Then, 1 OD₆₀₀*mL of the donor was added to 1 OD₆₀₀*mL of the recipient community or isolate and the mixture was plated on a 0.45 μm mixed cellulose ester membrane (Millipore) topping a plate of the recipient's preferred media without DAP. In the case of the thiocyanate bioreactor, —2 OD₆₀₀*mL of the donor was added to 2 OD₆₀₀*mL of the recipient community to ensure sufficient material despite the community's slow growth. Plates were incubated at the ideal temperature for the recipient community or isolate for 12 hours before the growth was scraped off the filter into the media of the recipient community or isolate for downstream analysis.

### ET-Seq Library Preparation

The insertion junction sequencing library prep strategy for ET-Seq can be used (modification may be necessary) in any circumstance where high efficiency mapping of inserted DNA to a host loci is desired. DNA of the edited community or isolate was first extracted using the DNeasy PowerSoil Kit (QIAGEN). In the case of the nine-member community, 500 ng of DNA was used for both insertion junction sequencing and metagenomic library prep. For the SCN community, which had lower yields of DNA, 100 ng were used [symbol] . As an internal standard, DNA from a previously constructed mutant library of Bacteroides thetaiotaomicron VPI-5482, a species not present in the nine-member community or the thiocyanate bioreactor, was spiked into the community DNA at a ratio of 1/500 by mass. The B. thetaiotaomicron library had undergone antibiotic selection for its transposon insertions and was thus assumed to represent 100% transformation efficiency (i.e. every genome contained at least one mariner transposon insertion).

For metagenomic sequencing, library prep was conducted by the standard ≥100 ng protocol from the NEBNext Ultra II FS DNA Library Prep Kit for Illumina (NEB). For insertion junction sequencing, the same protocol was used with a number of modifications enumerated here. This insertion junction sequencing protocol has also been tested successfully with the ≤100 protocol of the NEBNext Ultra II FS DNA Library Prep Kit (NEB) and the KAPA HyperPlus Kit (Roche). For fragmentation an 8 minute incubation was used. A custom splinklerette adaptor was used during adaptor ligation to decrease non-specific amplification. For size selection 0.15× (by volume) SPRIselect (Beckman Coulter, Cat #B23318) or NEBNext Sample Purification Beads (NEB) were used for the first bead selection and 0.15× (by volume) were added for the second. From this selection, the DNA was eluted in 44 uL (instead of the suggested 15 uL) where it undergoes digestion before enrichment to cleave intact transposon delivery vector. All bead elutions were performed with Sigma Nuclease-Free water. pHLL250 underwent AseI digestion, while DART vectors underwent double digestion by AsiSI and SbfI-HF (NEB). The DNA then underwent a sample purification using 1× AMPure XP beads (Beckman Coulter) to prepare it for PCR enrichment.

In PCR enrichment, the transposon junction was amplified by nested PCR. The PCRs followed the NEBNext Ultra II FS DNA Library Prep Kit for Illumina (NEB) PCR protocol, however in the first PCR the primers were custom to the transposon and the adaptor and the PCR was run for 25 cycles. The enrichment then underwent sample purification with a 0.7× size selection using SPRIselect or NEBNextSample Purification Beads from which 15 μL were eluted for the second PCR. This second PCR used custom unique dual indexing primers specific to nested regions of the insertion and adaptor and 6 cycles are used. Then another 0.7x size selection was conducted and the final library was eluted in 30 uL. Samples for metagenomic sequencing and insertion junction sequencing were then quality controlled and multiplexed using 1×HS dsDNA Qubit (Thermo Fisher) for total sample quantification, Bioanalyzer DNA 12000 chip (Agilent) for sizing, and qPCR (KAPA) for quantification of sequenceable fragments. Samples were sequenced on the iSeq100 or HiSeq4000 platforms.

### Genome Sequencing, Assembly, and Database Construction

Assembly and annotation of genomes used as references for the SCN bioreactor experiment is described in Huddy et al. (Huddy et al. 2020; bioRxiv. https://doi.org/10.1101/2020.04.29.067207. For genomes assembled as part of this study, cultures were grown on R2A medium for 24 hours at 30° C. and genomic DNA was extracted with the DNeasy Blood and Tissue DNA Kit (Qiagen) with pre-treatment for Gram-positive bacteria. Genomic DNA was sheared mechanically with the Covaris 5220 and processed with the NEBNext DNA Library Prep Master Mix Set for Illumina (NEB) before submitting for sequencing on an Illumina MiSeq platform generating paired end 150 bp reads.

Raw sequencing reads were processed to remove Illumina adapter and phiX sequence using BBduk with default parameters, and quality trimmed at 3′ ends with Sickle using default parameters (https:(//)github.com/najoshi/sickle). Assemblies were conducted using IDBA-UD v1.1.1 with the following parameters: -pre_correction -mink 30 -maxk 140 -step 10. Following assembly, contigs smaller than 1 kb were removed and open reading frames (ORFs) were then predicted on all contigs using Prodigal v2.6.3. 16S ribosomal rRNA genes were predicted using the 16SfromHMM.py script from the ctbBio python package using default parameters (https: (//)github.com/christophertbrown/bioscripts). Transfer RNAs were predicted using tRNAscan-SE. The full metagenome samples and their annotations were then uploaded into our in-house analysis platform, ggKbase, where genomes were manually curated via the removal of contaminating contigs based on aberrant phylogenetic signatures (https: (//)ggkbase.berkeley.edu).

For each ET-Seq experiment a genomic database is constructed using the ETdb component of the ETsuite software package. Each database contains the nucleotide sequences of the expected organisms in a sample, any vectors used, any conjugal donor, and the spike in control organism. For details on ETdb and database construction see (https: (//)app.gitbook.com/@sdiamond/s/etsuite/etdb/etdb). Briefly, all genomic sequences are formatted into a bowtie2 index to allow read mapping, a tabular correspondence table between all scaffold names and their associated genome is constructed, and a “genome info” table of standard genomic statistics is calculated including genome size, GC content, and number of scaffolds. Following database construction, a label is added to each entry in the genome info table manually to indicate if the entry represents a target organism, a vector, or a spike in control organism. All data are propagated into a single folder that can be used by the ETmapper software for downstream mapping and analysis.

### Identification and Quantification of Insertion Junctions and Barcodes

To identify and map transposon insertion junctions and their associated barcodes in a mixed population of microbial cells, reads (150 bp×2) generated from PCR amplicons of putative transposon insertion junctions are first processed using the ETmapper component of the ETsuite software package implemented in R with the following steps: First reads are quality trimmed at the 3′ end to remove low quality bases (Phred score ≥20) and sequencing adapters using Cutadapt v2.10. Cutadapt is then used to identify and remove provided transposon model sequences from the 5′ end of forward reads, requiring a match to 95% of the transposon sequence and allowing a 2% error rate. Read pairs where no transposon model sequence is identified in the forward read are discarded. All identified and trimmed transposon models are paired with their respective reads, stored, and barcodes are identified in these sequences by searching for a known primer binding site sequence flanking the 5′ end of the barcode (5′-CTATAGGGGATAGATGTCCACGAGGTCTCT-3′; SEQ ID NO:7) allowing for 1 mismatch. Subsequently, the 20 bp region following the known primer binding site is extracted as the barcode sequence and associated with its respective read. The 3′ end of the paired reverse reads are then trimmed to remove any transposon model sequence using Cutadapt, and only read pairs where one mate is at least ≥40 bp following all trimming are retained for downstream mapping and analysis. The fully trimmed paired end reads now consisting of only genomic sequence following the transposon insertion site are mapped to the ETdb database used in a given experiment using bowtie2 with default options. Mapped read files are converted into a hit table indicating the mapped genome, scaffold, genomic coordinates, mapQ score, and number of alignment mismatches for each read in a pair using a custom Python script, bam_pe_stats.py, provided with ETsuite. This table is then merged with read-barcode assignments to generate a final hit table with the mapping information about each read pair, the transposon model identified, and the associated barcode found for that read pair. Finally mapped read pairs filtered are only retained for downstream quantification if both reads map to the same genome, at least one mapped read in a pair has a mapQ score ≥20, and a barcode was successfully identified and associated with the read pair.

To quantify the number of unique barcodes and their associated reads mapping to organisms in each sample of an experimental run, the filtered hit tables were processed using the ETstats component of the ET-Seq software package with the following steps: Initially, all barcodes identified across all samples in an experiment are aggregated and clustered using Bartender with the following supplied options: -14 -s 1 -d 3. Barcode clusters and their associated barcodes/reads were only retained if all of the following criteria were true: (1) ≥75% of the reads in a cluster mapped to one genome (the majority genome), (2) ≥75% of the reads in a cluster were associated with the same transposon model (the majority model), and (3) the barcode cluster had at least 2 reads. Subsequently, when quantifying reads and barcodes in each sample of an experiment, the genome a read was mapped to and the transposon model it was associated with had to agree with the majority assignments for the barcode cluster assigned to that read's barcode to be counted. Finally, one were aware that Illumina patterned flow cell related index swapping would result in reads from a barcode cluster being misassigned across samples, even when using unique dual indexing. Barcode clusters could not simply be limited to be associated with only one sample, as our spike in control organisms contain the same pool of barcodes and are added to every sample. Thus, an empirical index swap rate was estimated across each experiment and required that the number of reads (X) for a barcode to be positively identified in a sample be always ≥2 and ≥ the binomial mean of observed read counts expected in any sample for a barcode cluster with (R) reads across (N) samples based on the estimated swap rate (S)+2 standard deviations (Eqn. 1).

$$X \geq ( R \times ( \frac{S}{N} ) ) + 2 \times \sqrt{R \times ( 1 - S ) \times S} \& X \geq 2 \qquad Eqn . 1$$

The index swap rate for an experiment was empirically estimated from barcode clusters assigned only to target organisms based on the assumption that it would be highly unlikely for a barcode cluster to have truly originated from independent integration events into the same organism in more than one sample. It was assumed that for each barcode cluster associated with target organisms, the majority of reads originated from the true sample and reads assigned to other samples represented swaps. This is opposed to barcode clusters associated with our spike in organism, conjugal donor organism, or vectors which contain the same pool of barcodes directly added to multiple samples. To identify swapped read counts, the total count of all reads assigned to the majority genome across barcode clusters but that are not associated with the majority sample of that cluster (E) was quantified. Then, the total count of reads associated with the majority genome was quantified and associated with the majority sample across all clusters (C). Then experiment wide swap rate was estimated by dividing the total number of reads not associated with majority samples by the total number of reads (Eqn. 2)

$$S = \frac{E}{( E + C )} \qquad Eqn . 2$$

Following filtering a hit table is returned that indicates for each genome in each sample, the number of unique barcode clusters that were recovered, and the total number of reads associated with these barcodes.

### Metagenomic Data Processing and Coverage Calculation

Each ET-Seq sample is split and in parallel undergoes shotgun metagenomic sequencing to determine the relative quantities of organisms present in the sample at the time of sampling. Raw read files from metagenomic data are also processed using the ETmapper component of the ETsuite software package with the following steps: First reads are quality trimmed at the 3′ end to remove low quality bases (Phred score ≥20) and sequencing adapters using Cutadapt v2.10. Read pairs where at least one mate is not ≥40 bp in length are discarded. Trimmed read pairs are mapped to the ETdb database used in a given experiment using bowtie2 with default parameters. Mappings are filtered to require a minimum identity ≥95% and minimum mapQ score ≥20, and coverage is calculated using a custom script, calc_cov.py, included with the ETsuite software.

### ET-Seq Normalization and Calculation of Insertion Efficiency

To account for differences in sequencing depth, transposon junction PCR template amount, and relative abundance of microbes in a community the data generated from both ET-Seq and shotgun metagenomics were each normalized independently to values from the spike in control organism, B. thetaiotaomicron, and then ET-Seq data is subsequently normalized by metagenomic abundance as follows: Initially read count tables from ET-Seq and metagenomics are filtered to remove any ET-Seq read count associated with <2 barcodes and any metagenomic read count <10 reads. Next a size factor for each sample is calculated based on the geometric mean of B. thetaiotaomicron reads for ET-Seq samples and B. thetaiotaomicron coverage for metagenomics samples. ET-Seq read counts and metagenomic coverage values are then divided by their respective sample size factors to create normalized values. Normalized ET-Seq read counts are then divided by their paired normalized metagenomic coverage values to generate ET-Seq read counts that are fully normalized to both ET-Seq sequencing depth and metagenomic coverage. Finally fully normalized ET-Seq read counts for target organisms are divided by the fully normalized ET-Seq read count of B. thetaiotaomicron from an experiment (a constant that represents the number of reads that would be obtained from an organism with 100% of its chromosomes carrying insertions). The resulting values for each target organism in a sample represent an estimate of the fraction of that organism's population that received insertions (Per Organism Insertion Efficiency). Additionally, a target organism's insertion efficiency was multiplied by the fractional relative abundance of that organism in a sample, based on metagenomic data, to estimate the fraction of an entire sample population that is made up of cells of a given species that received insertions (Per Community Insertion Efficiency).

### ET-Seq Validation and Establishing Limits of Detection and Quantification

To validate ET-Seq and establish both a limit of detection (LOD) and limit of quantification (LOQ) for the assay, a library of K. michiganensis transposon mutants was constructed by antibiotic selection following conjugation with pHLL250 (as described above), and this library was added to untransformed samples of the combined 9-member community to create a transformed cell concentration gradient. Technical triplicate samples were created where 1%, 0.1%, 0.01%, 0.001% and 0% of the total K. michiganensis cells (by OD₆₀₀) in the mixture were those derived from the transformed library. All samples (n=15) were subjected to ET-Seq (as described above), and pooled samples across all concentrations for each technical triplicate (n=3; 5 concentrations) were analyzed for community composition using shotgun metagenomics (as described above). ET-Seq per organism insertion efficiency values and per community insertion efficiency values were averaged across technical replicates. Additionally, to derive the fraction of transformed K. michiganensis cells that made up the total community (not just the K. michiganensis sub-population), the known fraction of K. michiganensis cells that were transformed in a sample was multiplied by the measured relative abundance of K. michiganensis in a given technical replicate, and these values were averaged across technical replicates.

To derive the LOD and LOQ for ET-Seq a linear regression was performed using the lm function in the base package of R using the known fraction of transformed K. michiganensis cells that made up the total community as the independent variable and the ET-Seq estimated per community insertion efficiency as the dependent variable. The sample where transformed K. michiganensis made up 0% of the community was not included in the regression analysis, but was reserved to demonstrate zero response with no transformed cells present. LOD was calculated as 3.3*standard error of the regression/slope. The LOQ was calculated as 10*standard error of the regression/slope.

### Identification of Positive Transformations and Statistical Analysis

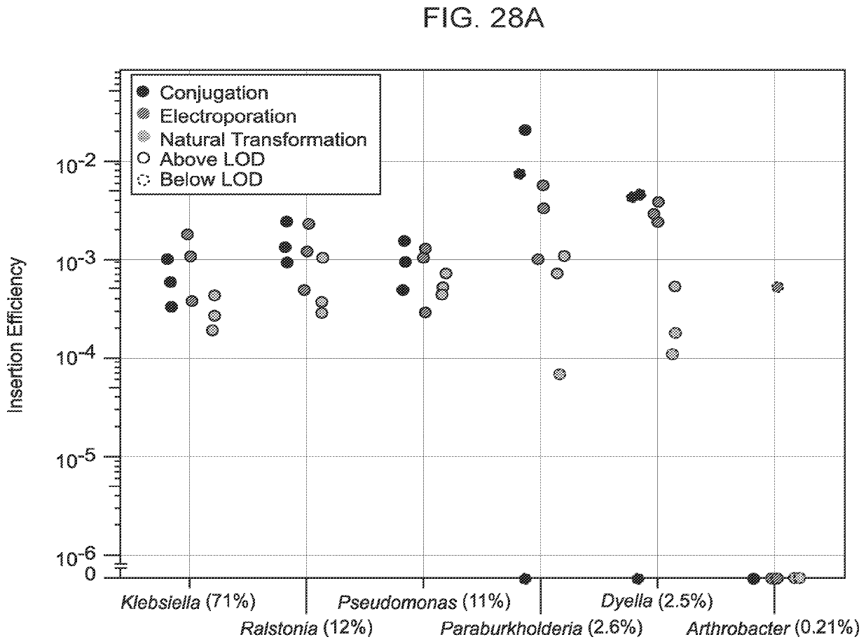

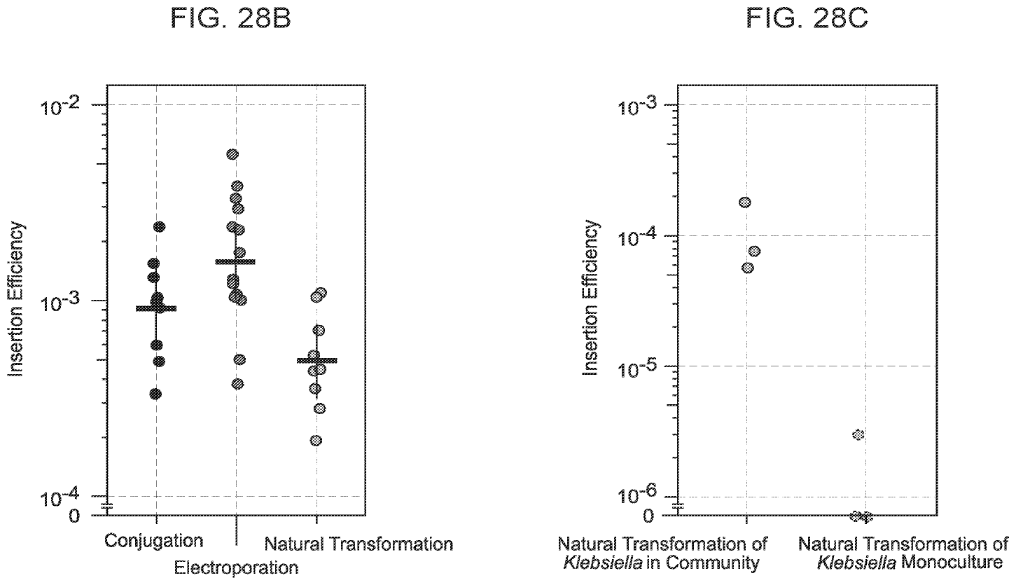

For all ET-Seq experiments conducted, it was initially determined if any ET-Seq estimated per community insertion efficiency was larger than the LOD. Values larger than the LOD constituted a positive detection. For comparative statistical analysis conducted to compare per organism insertion efficiencies between transformation methods (FIG. 28B) only values that had a corresponding per community insertion efficiency >LOQ were used. Statistical testing was conducted using Analysis of Variance (ANOVA) implemented in the aov function in R Post-hoc testing was conducted using the TukeyHSD function in R. Traditional 95% confidence intervals were calculated using the groupwiseMean function of the rcompanion package in R.

### Multiple Delivery Experiments in Communities

To test multiple delivery methods on the nine-member community, all members were grown at 30° C. with Bacillus sp. AnTP16 and Methylobacterium sp. UNC378MF in R2A liquid media while all other members were inoculated in LB. Equal amounts of community members were then combined by OD₆₀₀. This consortia then underwent transformation (of pHLL250), conjugation (of AMD290; pHLL250 in WM3064), and electroporation of the pHLL250 vector (described in Delivery Methods section). After delivery the community was spun down at 5,000 g for 10 minutes, washed once with LB and then spun down and frozen at −80° C. until genomic DNA extraction.

The thiocyanate degrading microbial community was sampled for delivery testing from biofilm on a four liter continuously stirred tank reactor that had been maintained at steady state for over a year. The reactor is operated with a two day hydraulic residence time, sparged with laboratory air at 0.9 L/min, and fed with a mixture of molasses (0.15% w/v), thiocyanate (250 ppm), and KOH to maintain pH 7. OD measurements were not feasible on the biofilm, so its wet mass was used to approximate equivalent OD and thus cell numbers to those used for the nine-member community. This community underwent the same transformation, electroporation, and conjugation delivery approaches as the nine-member community, however in all steps requiring media, LB was replaced with molasses media (no thiocyanate). After delivery the community was spun down at 5,000 g for 10 minutes, washed once with molasses media and then spun down and frozen at −80° C. until genomic DNA extraction.

Benchmarking DART Systems in E. coli

Several DART systems were constructed, to identify variants capable of efficient transposition by conjugative delivery to E. coli. Parallel conjugation were performed of each DART vector variant containing Gm^{R} Tn cargo (2.1 kbp) and either a non-targeting gRNA or one of two lacZ-targeting gRNAs for each system. For VcDART, variation of the promoter controlling the expression of VcCasTn components did not significantly impact transposition efficiency. Similarly for ShDART, expression of the sgRNA in three distinct transcriptional configurations did not significantly impact transposition efficiency. Since promoter and transcriptional configuration variation had insignificant effects on transposition efficiency—and to remove the requirement for promoter induction and reliance on T7 RNA polymerase—target specificity benchmarking of VcDART and ShDART was performed using the same constitutive P_{lac} promoter. In this experiment, ShDART Cas and Tns genes and sgRNA were encoded in the original transcriptional configuration and under control of the same promoter in which ShCasTn was first characterized by Strecker et al. (Strecker et al. 2019).

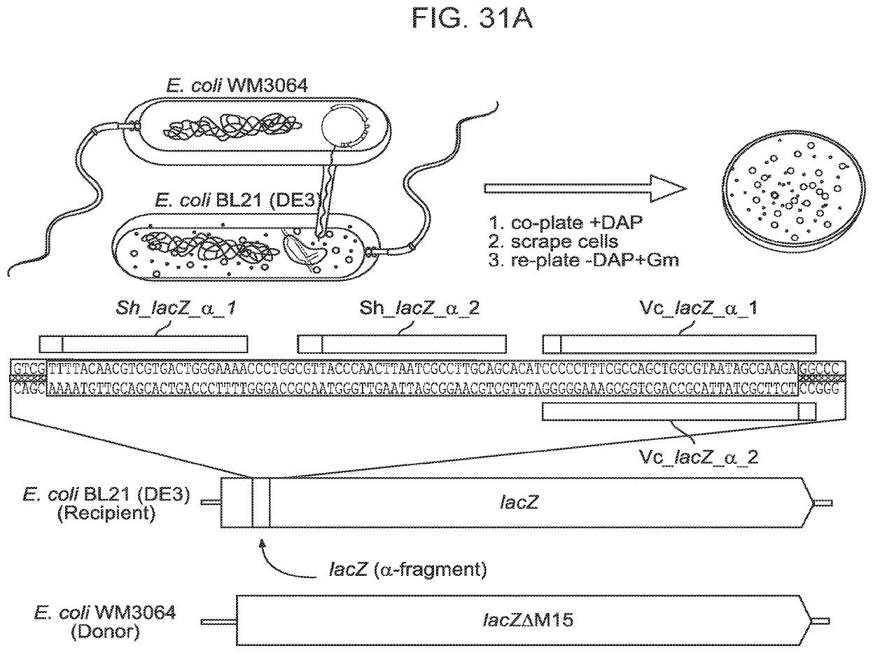

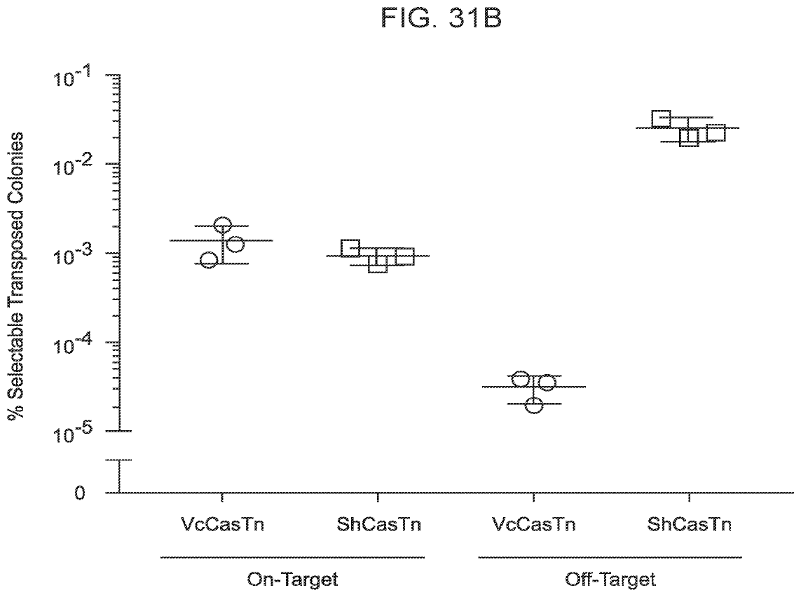

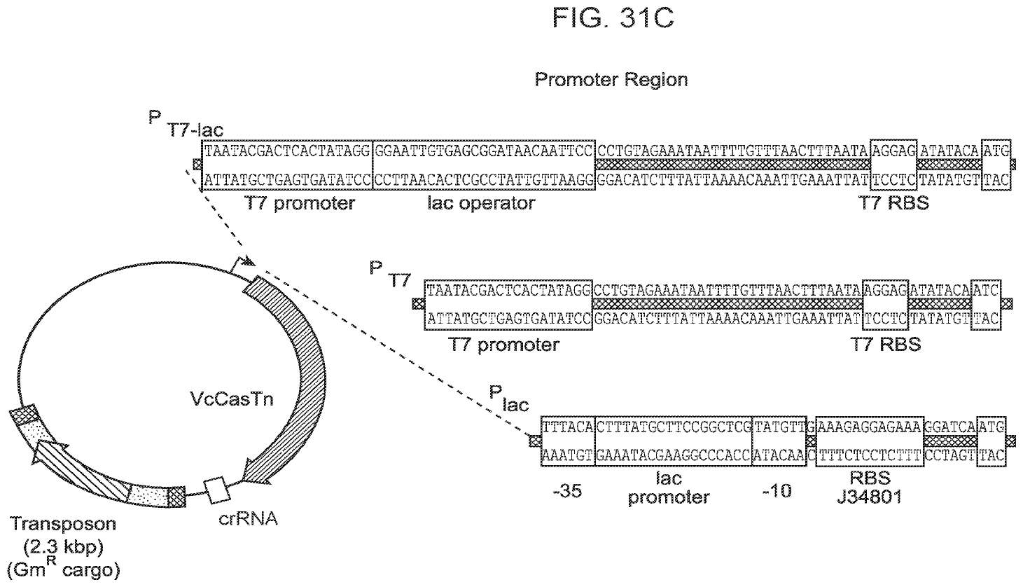

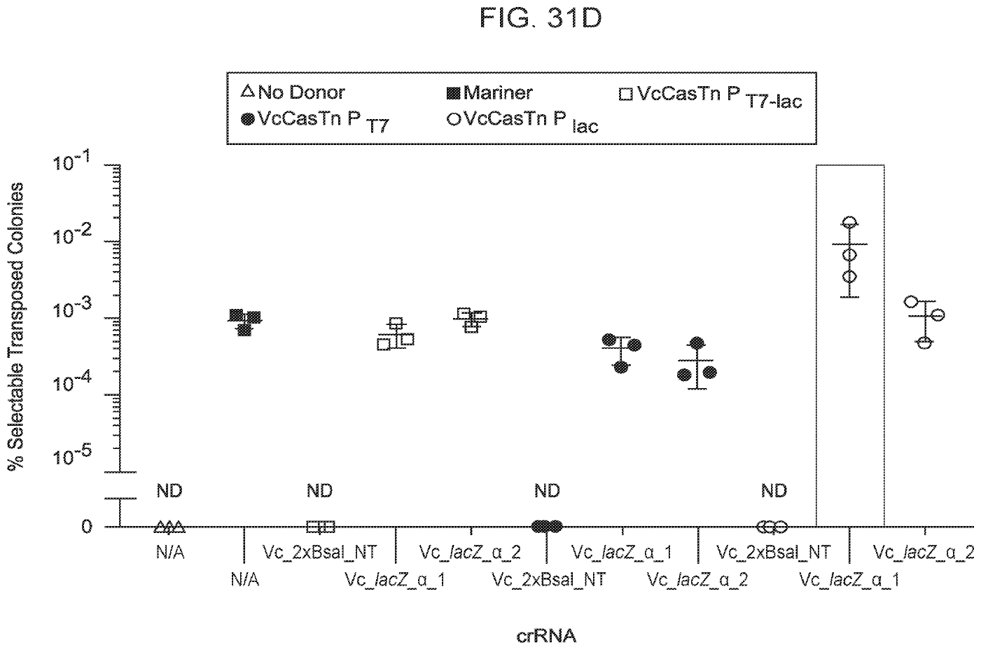

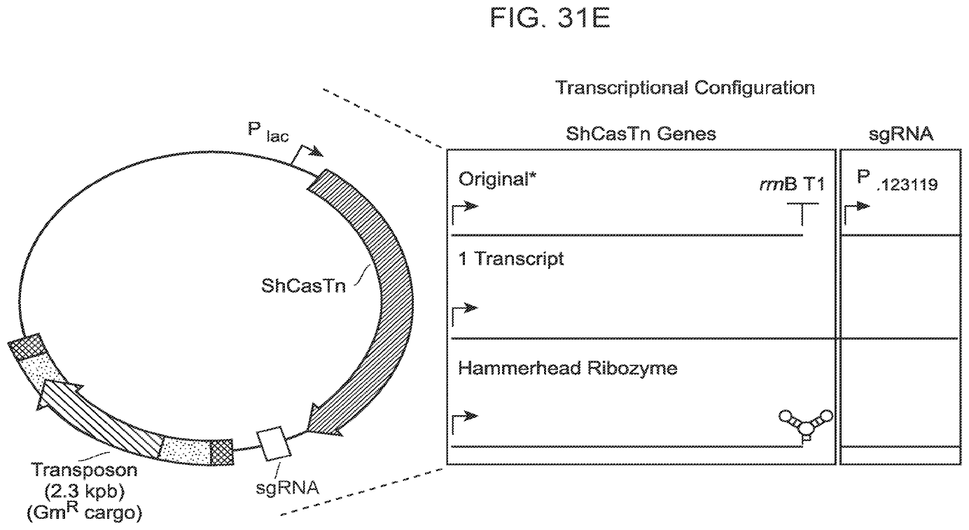

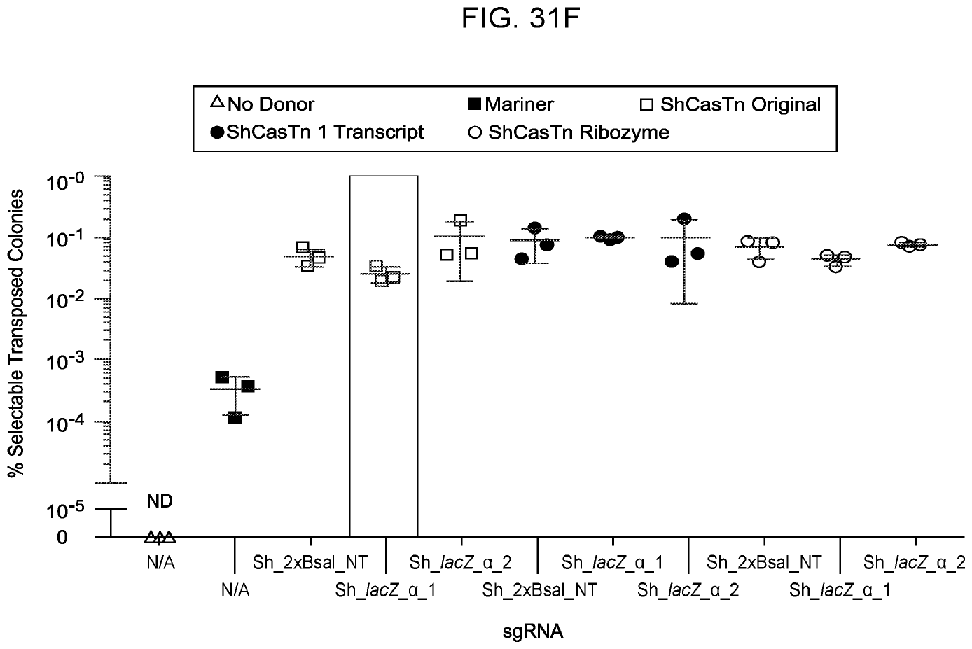

The lacZ-targeting gRNAs were designed to target the lacZ α-peptide present in the conjugation recipient strain E. coli BL21(DE3) but absent in the lacZΔM15 strains used as cloning host (E. coli EC100D-pir+) or conjugation donor (E. coli WM3064), preventing transposition until delivery into the recipient cell (FIG. 31A). Donor WM3064 strains were transformed and cultivated as described above, and recipient BL21(DE3) was inoculated from glycerol stock into 100 mL LB in a 250 mL baffled shake flask at 37° C. 250 rpm. Conjugations were performed as described above using LB medium and 37° C. incubation for every step, except that 0.1 mM IPTG was added to VcDART conjugation plates to induce transcription from P_{T7 lac} and T7 RNA polymerase expression in E. coli BL21(DE3). Transposition efficiencies were calculated as the percentage of colonies resistant to 10 μg mL gentamycin relative to viable colonies in absence of gentamycin.

On/off-target analysis was performed for one lacZ-targeting guide for each DART system by outgrowth under selection followed by genomic DNA extraction and ET-Seq. Specifically, approximately 10,000 transconjugant cfu were plated on LB agar with gentamycin, incubated at 37° C. overnight, scraped from agar into liquid LB medium, diluted to OD₆₀₀=0.25 into 10 mL LB plus gentamycin in 50 mL conical tubes, incubated at 37° C. 250 rpm until OD₆₀₀=1.0, centrifuged at 4,000 g, and frozen for downstream analysis. To determine the percent of selectable transposed colonies possessing on-target and off-target edits, the total number of selectable colonies was adjusted (for on-target and off-target percent as determined by ET-Seq.

### VcDART-Mediated Targeted Editing Isolation in a Community

VcDART vectors encoding constitutive VcCasTn, constitutive bla:aadA Tn cargo (2.7 kbp), and either a non-targeting (pBFC0888), K. michiganensis M5aI pyrF-targeting (pBFC0825), or P. simiae WCS417 pyrF-targeting (pBFC0837) constitutive crRNA were transformed into E. coli WM3064. Conjugations of these vectors into the nine-member community were performed as described above on filter-topped LB agar plates with 12 hr incubation at 30° C. Lawns were scraped from filters into 10 mL LB medium, vortexed, and 1 OD₆₀₀*mL from each lawn was plated on LB agar supplemented with 1 mg mL 5-FOA, 100 ug mL⁻¹ carbenicillin, 100 ug mL⁻¹ streptomycin, and 100 ug mL⁻¹ spectinomycin. Following 3 days of incubation at 30° C., all cells were scraped from the agar into 10 mL R2A medium, vortexed, diluted into 10 mL R2A supplemented with 20 mg mL⁻¹ uracil (for no selection controls) or R2A with uracil, 5-FOA, carbenicillin, streptomycin, and spectinomycin to OD₆₀₀=0.02, and split evenly across 4 wells (2.5 mL/well) of a 24 deep well plate. After cultivation at 30° C. and 750 rpm for 1 week, only the cultures conjugated with VcDART containing pyrF-targeting crRNA had grown in presence of antibiotics and 5-FOA. A small portion of each of these cultures was serially diluted in R2A and plated on LB agar plus antibiotics to isolate and assay colonies by targeted PCR and Sanger sequencing of pyrF loci. The remainder of each culture was centrifuged at 4,000 g for 10 min and frozen at −80° C. for downstream bacterial 16S rRNA V4 amplicon metagenomic sequencing (Novogene). Relative abundances were calculated as described above for pre-conjugation nine-member community cultures and post-selection pyrF-targeted cultures.

### Community Targeted Fitness Assay

The output reads generated by the ETstats script from the ETsuite pipeline, were filtered for read clusters that show greater than 80% purity based on the Bartender output. Bartender assigns purity to barcode clusters based on the fraction of reads associated with the cluster that map to the same genomic region. The filtered ETstats output were then converted to a bed file format and the number of unique barcodes or reads that map to the genome within a 200 bp window of the VcDART target site were identified using Bedtools. Quinlan and Hall (2010) Bioinformatics 26:841. For the genome-wide targeting plot, the respective genomes were divided into 500 bp bins and the frequency of reads from the ETstats output mapping to each bin were calculated using Bedtools.

### Statistics and Reproducibility

All transformations (natural transformation, conjugation, electroporation) and subsequent analyses were performed for three independent replicates. In some cases, gel and plate images show single replicates that are representative of all replicates.

### Results

### ET-Seq Detects Genetically Accessible Microbial Community Members

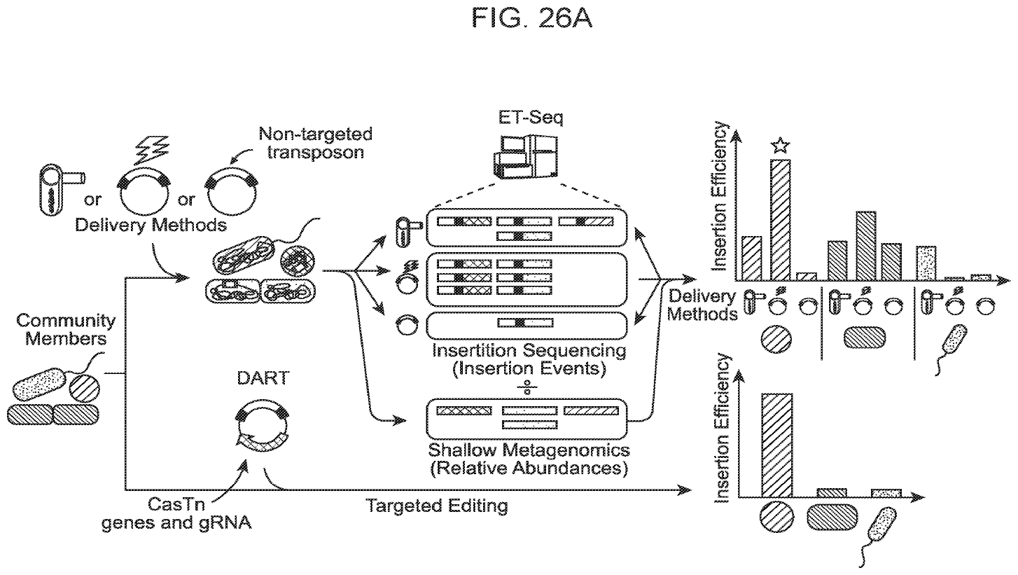

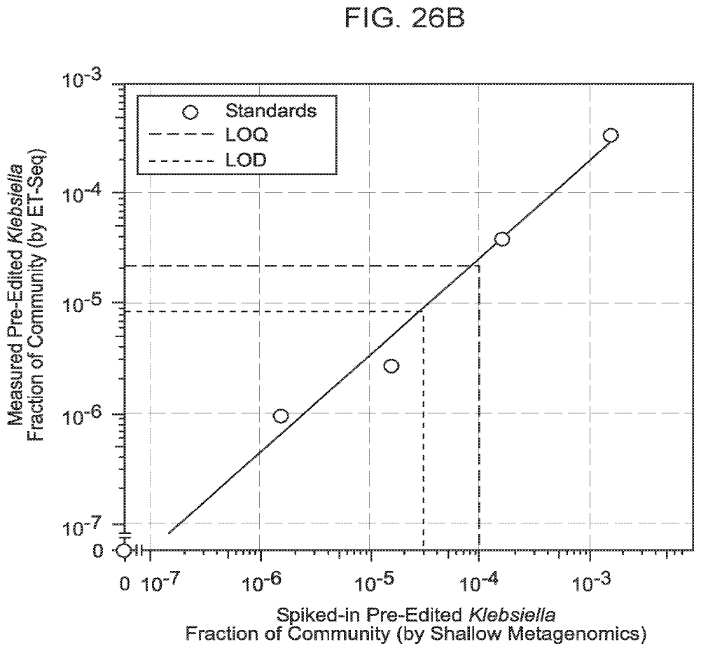

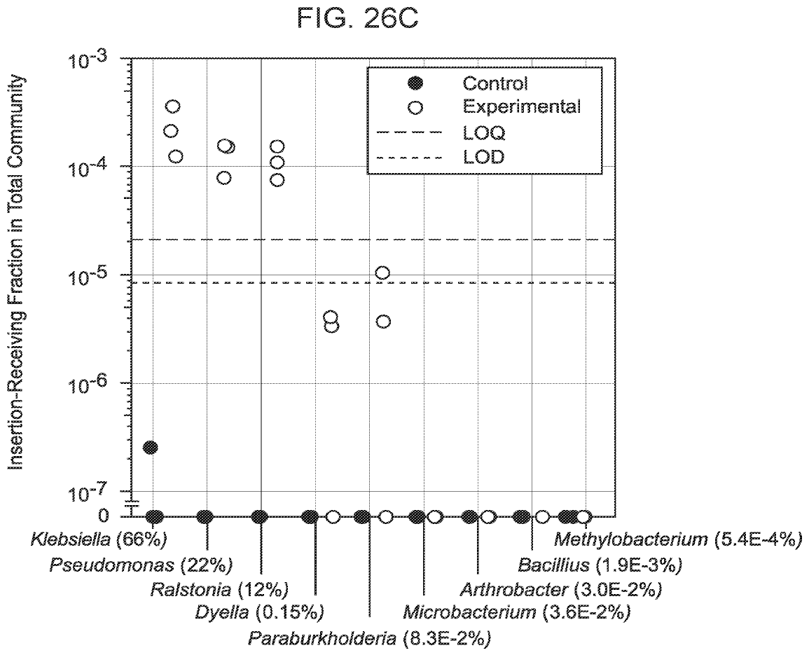

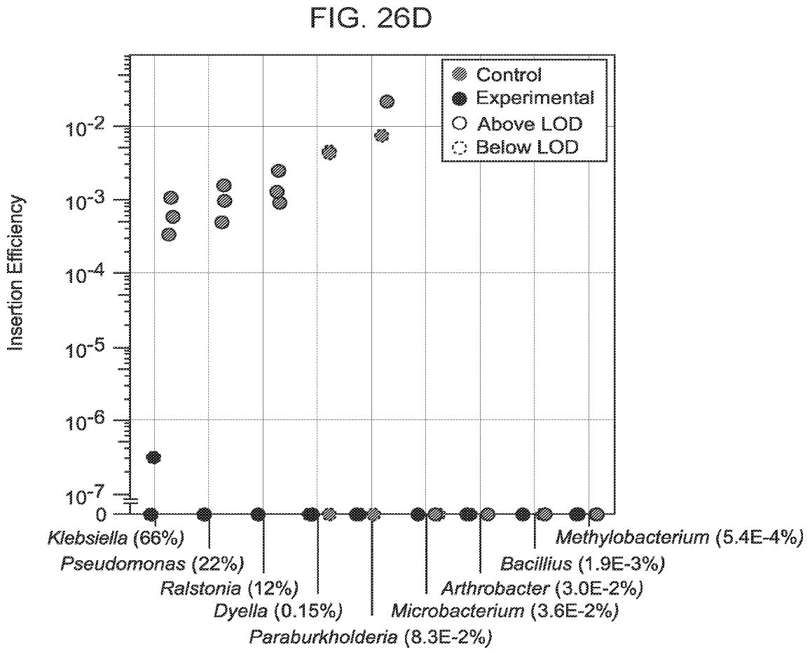

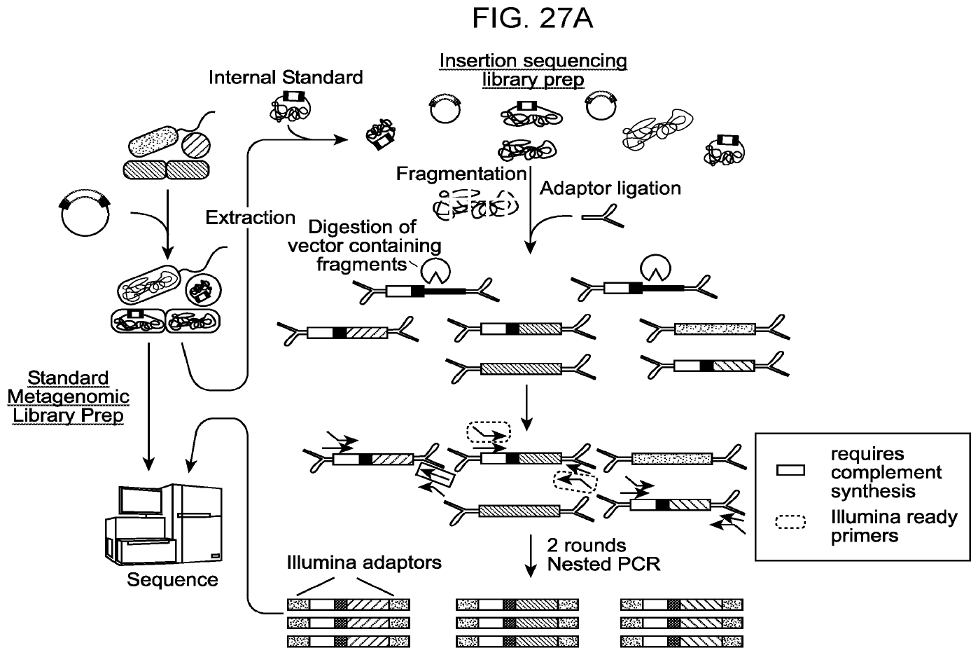

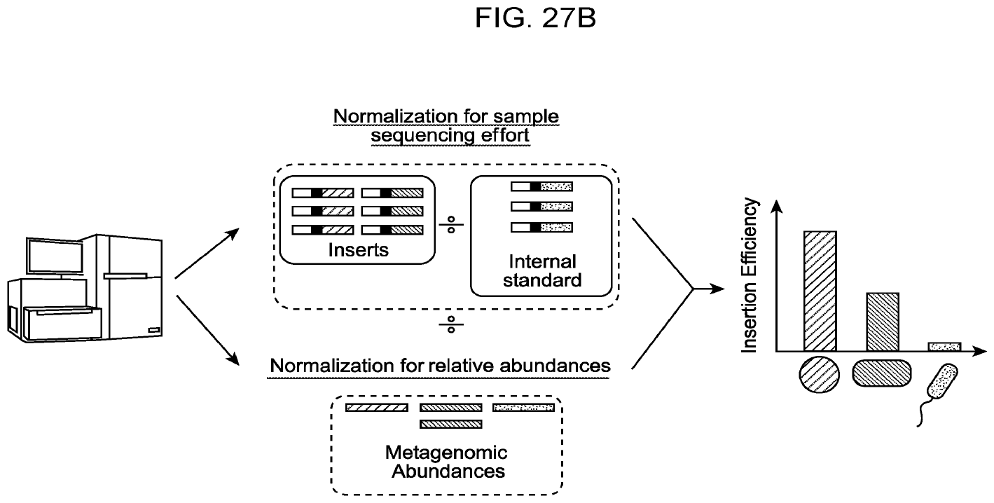

A prerequisite for editing a complex microbiome is to determine which constituents are genetically accessible in the community context and with what efficiency. To achieve this, ET-Seq was developed to assay the ability of community members to take up and integrate exogenous DNA (FIG. 26A). In ET-Seq, a microbial community is exposed to a randomly integrating mobile genetic element (here, a mariner transposon), and in the absence of any selection, total community DNA is then extracted and sequenced using two protocols. In the first, the junctions between the insertion and host DNA were enriched and sequenced, to determine insertion location and quantity in each host. This step requires comparison of the junctions to previously sequenced community reference genomes. In the second, low-depth metagenomic sequencing was conducted to quantify the abundance of each community member in a sample. Together, these sequencing procedures provide relative insertion efficiencies for microbiome members. To normalize these data according to a known internal standard, to every sample a known amount was added of genomic DNA from an organism that has previously been transformed with, and selected for, a mariner transposon. In the internal standard, it is expected that every genome contains an insertion. The final output of ET-Seq is a fractional number representing the proportion of a target organism's population that harbored transposon insertions at the time DNA was extracted, or insertion efficiency (FIG. 27A). The final output of ET-seq then returns a fraction that represents the proportion of a target organism's population that harbored transposon insertions at the time DNA was extracted. To facilitate the analysis of these disparate data, a complete bioinformatic pipeline was developed for quantification of insertions and normalization by both spike in control and metagenomic abundance (https://at github(dot)com/SDmetagenomics/ETsuite and Methods). Together these approaches allow for the determination of genetic accessibility, by measuring the percentage of each well represented member of a given microbiome receiving insertions (FIG. 27B).

ET-Seq was developed and tested on a nine-member microbial consortium made up of bacteria from three phyla that are often detected and play important metabolic roles within soil microbial communities. An initial effort was made to test the accuracy and detection limit by adding to the nine-member community a known amount of a previously prepared mariner transposon library of one of its member species, Klebsiella michiganensis M5a1. The ET-Seq derived insertion efficiencies were closely correlated to the known fractions of edited K. michiganensis present in each sample (FIG. 26B). Using this data, a limit of detection (LOD) and limit of quantification (LOQ) was calculated for the method, which suggest that a fraction of ≥8.4*10⁶ of transformed cells out of the total community would be detectable by ET-Seq.

Next, the mariner transposon vector was delivered to the nine-member community through conjugation. Conjugation could be measured reproducibly and quantitatively in the three species that grew to make up over 99% of the community (FIG. 26C). Insertion efficiency was further normalized as a portion of the whole community by relative abundance of each community member to get transformation efficiencies for each organism (FIG. 26D). Even for Paraburkholderia bryophila 376MFSha3.1 and Dyella japonica UNC79MFTsu3.2, which each made up approximately 0.1% of the community, delivery and insertion could be measured, but with lesser confidence. Although other community members showed no insertions, whether this is because of extreme rarity in the community or recalcitrance to delivery and insertion cannot be concluded.

FIG. 26A-26D. ET-Seq for quantitative measurement of non-targeted editing in a microbial community. a, ET-Seq provides data on insertion efficiency of multiple delivery approaches, including conjugation, electroporation, and natural DNA transformation, on microbial community members. In this illustrative example, the blue strain is most amenable to electroporation (star). This data allows for the determination of feasible targets and delivery methods for DART targeted editing. b, ET-Seq determined efficiencies for known quantities of spiked-in pre-edited K. michiganensis. Data shown is the mean of three technical replicates. LOD is the lowest insertion fraction at which accurate detection of insertions is expected and LOQ is the lower limit at which this fraction is expected to be quantifiable. c-d, ET-Seq determined insertion efficiencies in the nine-member consortium with conjugative delivery shown as c, a portion of the entire community and d, a portion of each species. Control samples received no DNA delivery. Relative abundances of community constituents are indicated in parentheses.

FIG. 27A-27B. Library preparation and data normalization for ET-Seq. a, ET-Seq requires low-coverage metagenomic sequencing and customized insertion sequencing. Insertion sequencing relies on custom splinkerette adaptors, which minimize non-specific amplification, a digestion step for degradation of delivery vector containing fragments, and nested PCR to enrich for fragments containing insertions with high specificity. The second round of nested PCR adds unique dual index adaptors for Illumina sequencing. b, This insertion sequencing data is first normalized by the reads to internal standard DNA which is added equally to all samples and serves to correct for variation in reads produced per sample. Secondly, it is normalized by the relative metagenomic abundances of the community members.

ET-Seq was further expanded to compare insertion efficiencies in the nine-member community by several common delivery techniques: conjugation, natural transformation with no induction of competence, and electroporation of the transposon vector. Together these approaches showed reproducible insertion efficiencies above the limit of detection (LOD) in five of the nine community members (FIG. 28A). Additionally, preferred delivery methods were identified for some members in this community context, such as electroporation likely being effective for Dyella japonica UNC79MFTsu3.2 while conjugation was not. These results show that ET-Seq can identify and quantify genetic manipulation of microbial community members and reveal suitable DNA delivery methods for each.

Notably, five organisms exhibited some degree of natural competency, although average efficiencies were significantly lower for natural transformation than for delivery through electroporation (ANOVA; p=0.0009) (FIG. 28B). No representatives of the Klebsiella genus including K. michiganensis are known to be naturally competent. A second ET-Seq experiment was conducted to compare the insertion efficiency of K. michiganensis in isolation to its efficiency in a community context. ET-Seq returned no values above the LOD for natural transformation of K. michiganensis in isolation, but within the community ET-Seq returned values well into the quantifiable range (FIG. 28C). Although previous work has suggested the possibility of community effects on natural competence (Borgeaud et al. (2015) Science 347 (6217): 63-67), the evidence is minimal due to lack of tools for measuring horizontal gene transfer (HGT) events within a community. Therefore, it was noteworthy to observe emergent properties of natural transformation in such a small synthetic consortium of isolates.

FIG. 28A-28C. ET-Seq detection of insertion efficiency across multiple delivery approaches. a, ET-Seq determined insertion efficiencies for conjugation, electroporation, and natural transformation on the nine-member consortia. Only members with at least one positive insertion efficiency value across the delivery methods are shown. b, Comparing delivery strategies across data from all organisms c, Comparing natural transformation in isolate K. michiganensis compared to K. michiganensis in the community context.

Genetic Accessibility of Uncultivated Species within an Environmental Microbiome

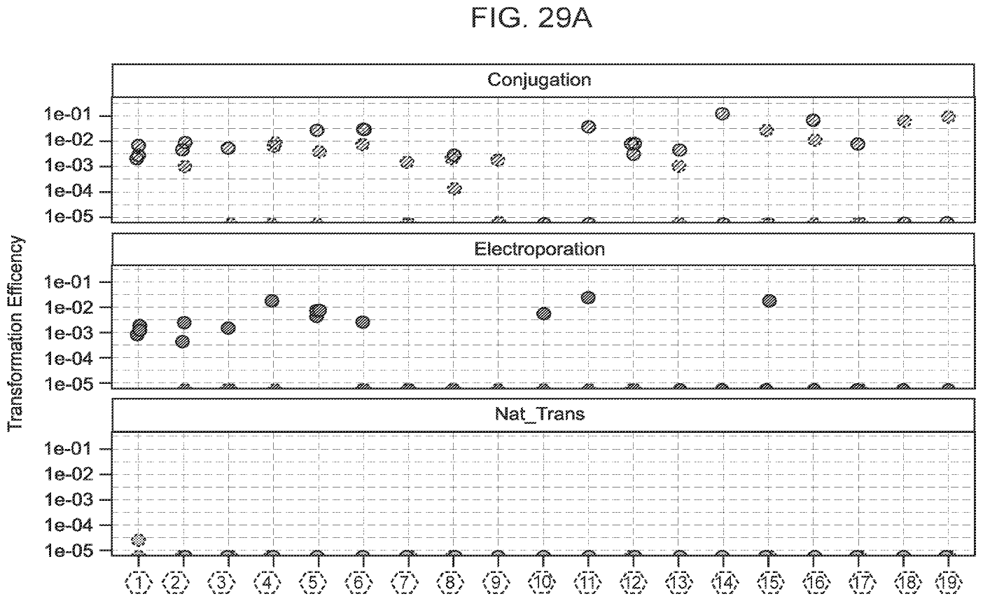

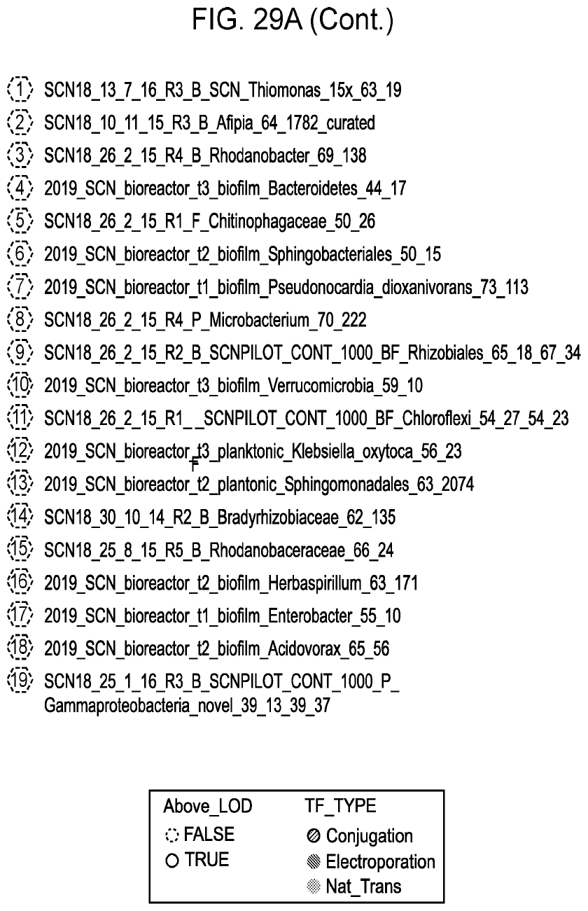

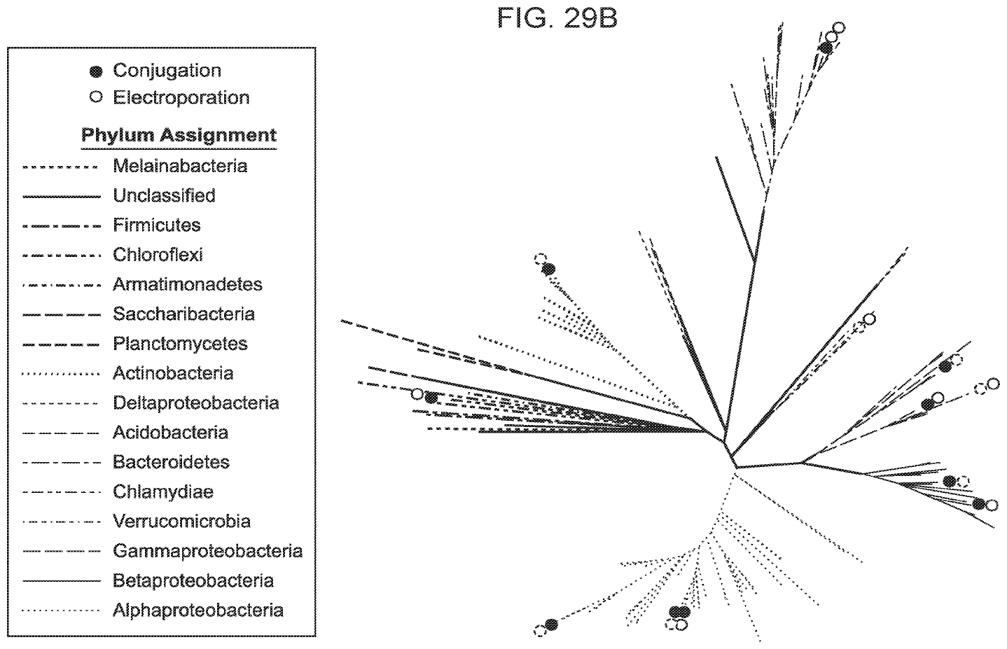

To map the potential for editing of a complex and environmentally realistic community that has not been reduced to isolates, ET-Seq was conducted on a genomically characterized 197 member bioreactor-derived consortia that degrades thiocyanate (SCN) (Kantor et al. (2017) Environmental Science & Technology 51 (5): 2944-53). Thiocyanate, a toxic compound produced from cyanide during gold processing, can be metabolized into its non-toxic components by this reactor community. Biofilm was sampled from the reactor and ET-Seq was conducted with a panel of delivery techniques: conjugation, electroporation, and natural transformation. ET-Seq showed at least one measurement of insertions above detection limit in 15 members of the bioreactor community (FIG. 29A). Ten of these were from species which had not previously been isolated or edited; and overall members from 5 of the 12 phyla detected in this consortium were successfully transformed (FIG. 29B). This included an Afipia sp. known to play an important role in the thiocyanate degradation process. Notably, members of the CPR are resistant to typical isolation techniques due to heavy dependence on other community members, and little is known about the nature of their likely symbiotic relationships with other organisms. Here, ET-Seq has uncovered a genetically tractable putative host organism, raising the possibility of genetically editing the host to probe CPR/host symbiotic relationships within a complex microbial community. In this way, ET-Seq reveals genetic accessibility and the tools necessary to achieve it in previously unapproachable and biologically important members of an environmentally relevant community.

FIG. 29A-29B. ET-Seq detection of insertion efficiency in thiocyanate-degrading bioreactor. a, ET-Seq determined editing efficiencies for conjugation, electroporation, and natural transformation on the thiocyanate bioreactor community. b, Members receiving insertions by conjugation or electroporation shown across a phylogenetic tree of all organisms in the thiocyanate bioreactor. Tree was constructed from an alignment of 262 rps3 protein sequences using IQtree.

### Targeted Genome Editing in Microbial Communities Using CRISPR-Cas Transposases

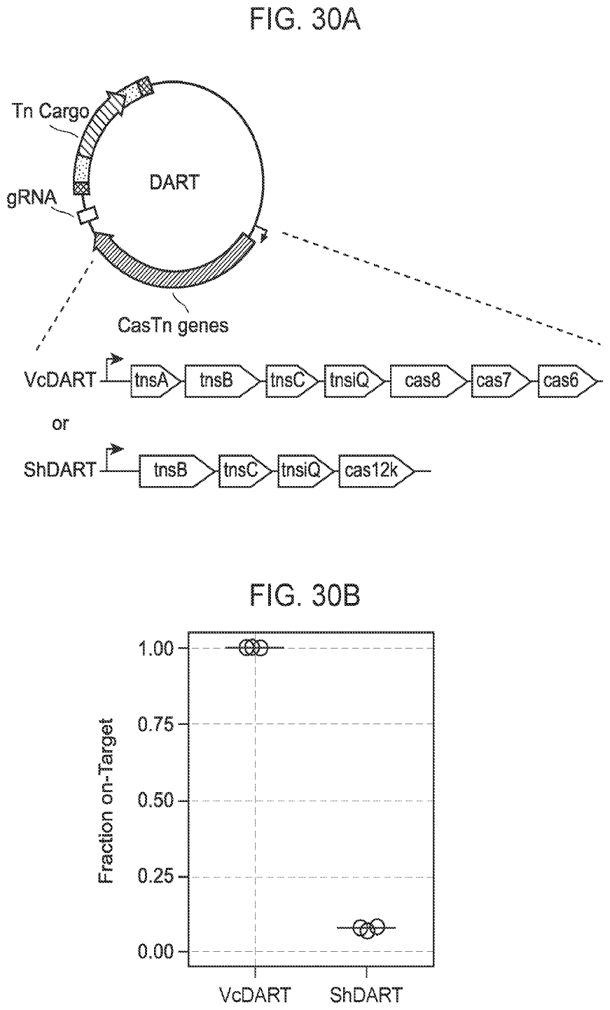

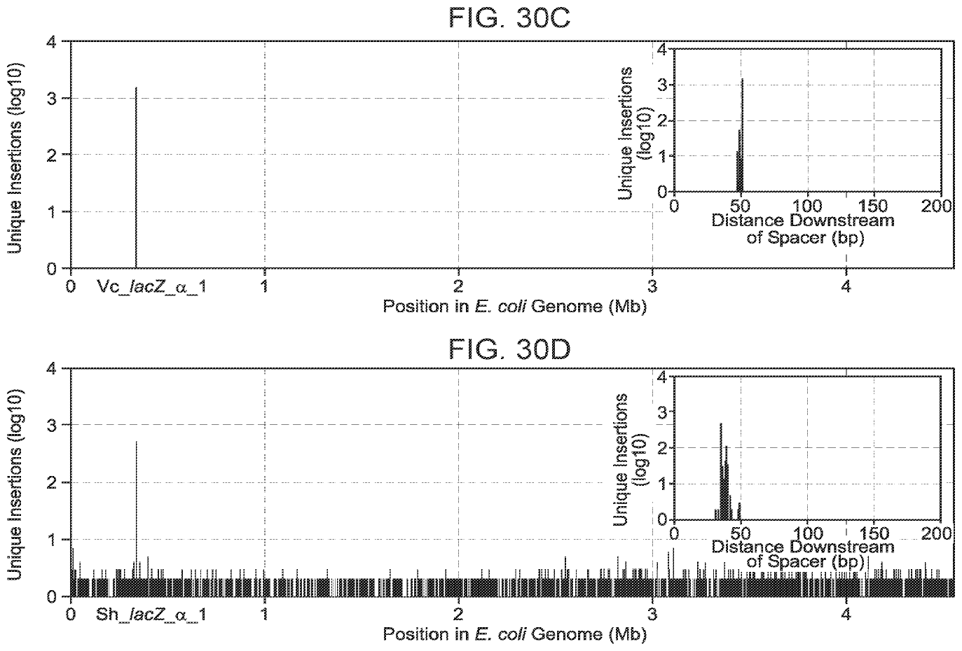

Genome edits that are both specific to a single organism in a microbial community and targeted to a defined location in its genome will be required to expose inter-species interactions and to enable molecular genetics in the uncultured majority of microbial life. It was reasoned that RNA-guided CRISPR/Cas Tn7 transposases would provide the ability to both ablate function of targeted genes and deliver customized genetic cargo in organisms shown to be tractable by ET-Seq (FIG. 26A). However, the two-plasmid ShCasTn (Strecker et al. (2019) Science 365 (6448): 48-53) and three-plasmid VcCasTn (Klompe et al. (2019) Nature 571 (7764): 219-25) are not amenable to efficient delivery beyond E. coli or within communities. Since ET-Seq identified conjugation and electroporation as broadly effective delivery approaches in the tested communities, all-in-one conjugative versions of these CasTn vectors that could be used for delivery by either strategy were designed and constructed (FIG. 30A). These DNA-editing All-in-one RNA-guided Transposase (DART) systems are barcoded and compatible with the same sequencing methods used for ET-Seq, and can be used to assay the efficacy of CRISPR-Cas-guided transposition into the genome of a target organism.

The transposition efficiency and specificity of the DART systems were compared in E. coli in order to select the most promising candidate for targeted genome editing in microbial communities. VcDART and ShDART systems harboring Gm^{R} cargo with a lacZ-targeting or non-targeting guide were conjugated into E. coli to quantify transposition efficiency, and target site specificity was assayed using ET-seq following outgrowth of transconjugants in selective medium (FIG. 31A). While VcDART and ShDART yielded a similar number of selectable colonies possessing on-target insertions, >96% of the selectable insertions obtained using ShDART were off-target compared to <4% for VcDART (FIG. 30A-30D; and FIG. 31B). Due to VcDART's high target site specificity, developed this system was further developed for targeted community editing.

FIG. 30A-30D. Benchmarking all-in-one conjugal targeted vectors. a, Schematic of VcDART and ShDART delivery vectors. b, Fraction of insertions that occur in a 200 bp window around the target site. Mean for three independent biological replicates is shown. c-d, Unique insertion counts across the E. coli genome using c, VcDART and d, ShDART.

FIG. 31A-31F. Benchmarking all-in-one conjugal CasTn vectors. a, E. coli WM3064 to E. coli BL21(DE3) conjugation, transposition, and selection schematic (top) and guide RNAs targeting the α-fragment of recipient BL21(DE3), which is absent from donor WM3064 (bottom). b, VcDART on:off-target transposition frequency exceeds that of ShDART, which yields far more off-target than on-target transpositions. % selectable transposed colonies is calculated as percent of colonies obtained with gentamycin selection relative to total viable colonies in absence of selection. On- and off-target percentages in FIG. 30C are multiplied by % selectable transposed colonies to obtain the plotted values. Guide RNAs used here (Vc_JacZ_α_1 and Sh_JacZ_α_1) are highlighted with gray bands. c, Transposition with VcDART was tested with three promoters. d, Efficiencies of all-in-one VcCasTn using various promoters. e, Transposition with all-in-one ShCasTn was tested with three transcriptional configurations, all using P_{lac}. f, Efficiencies of all-in-one ShCasTn using various promoters. For all plots, data represents mean and one standard deviation for three independent biological replicates, and guide RNAs ending in “NT” are non-targeting negative control samples.

### Targeted Microbial Community Editing by Programmable Transposition

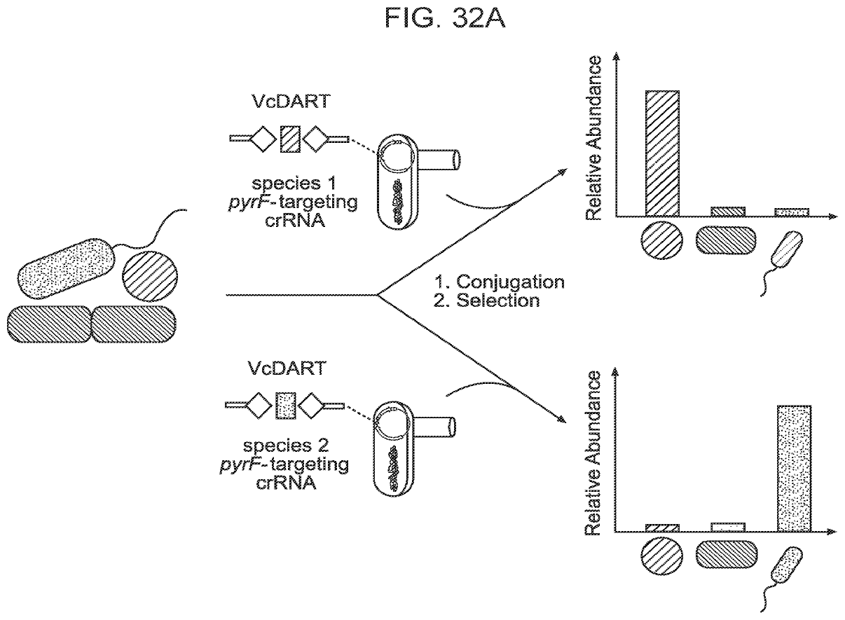

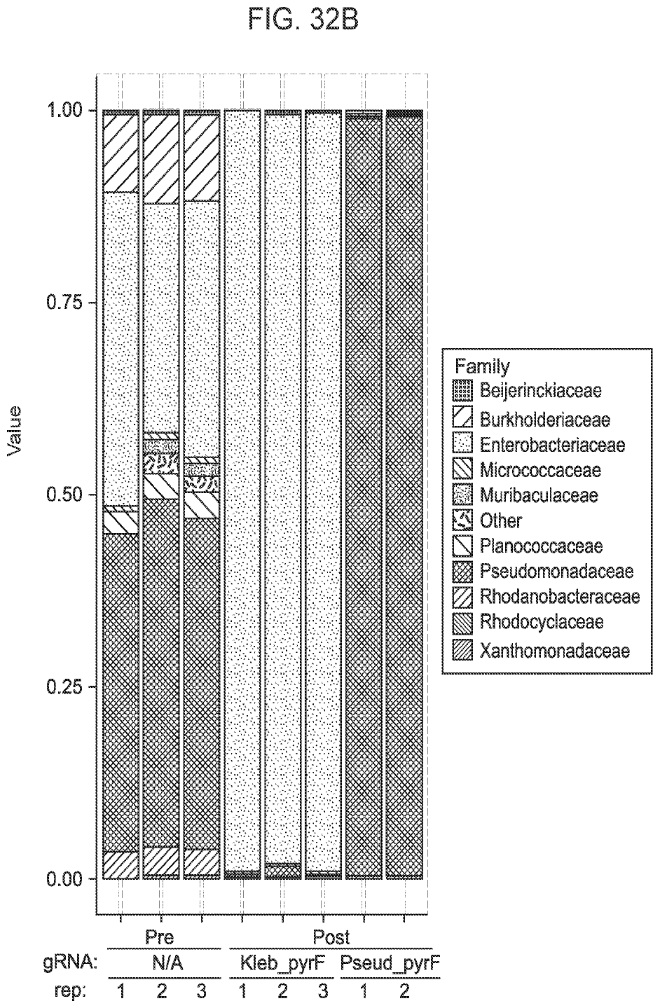

RNA-programmed transposition was used for targeted editing of a microbial consortium. ET-Seq had shown the members of the nine-member community, K. michiganesis and Pseudomonas simiae WCS417, to be both abundant and tractable by conjugation (FIG. 26C). In parallel, both of these organisms were targeted by conjugation of the VcDART vector into the community with guides specific to their genomes. To validate editing, the insert was used as a “hook” to isolate the targeted members from the community (FIG. 32A). Insertions were designed to produce loss-of-function mutations in the K. michiganesis and P. simiae pyrF gene, an endogenous counterselectable marker allowing growth in the presence of 5-fluoroorotic acid when disrupted. The transposons carried two antibiotic resistance markers conferring resistance to streptomycin and spectinomycin (aadA) and carbenicillin (bla). Together the simultaneous loss-of-function and gain-of-function mutations allowed for a strong selective regime. VcDART targeting to K. michiganensis and P. simiae pyrF and selection led to targeted enrichment to >99% pure culture for each target organism, while no outgrowth was detected when using a non-targeting guide RNA (FIG. 32B). K. michiganensis and P. simiae colonies further verified by PCR and Sanger sequencing showed full length, pyrF-disrupting VcDART transposon insertions 48-49 bp downstream of the guide RNA target site. In this way, simply reprogramming a 32 bp gRNA allows for the targeted editing and isolation of distinct members of a microbial community. These results demonstrate that targeted genome editing using DART enables genetic manipulation of distinct members of a complex microbial community. This targeted editing of microorganisms in a community context can also enable subsequent exploitation of modified phenotypes.

FIG. 32A-32B. Targeted editing in the 9-member consortium. a, Conjugative VcDART delivery into a microbial community using species-specific crRNA, followed by selection for transposon cargo, facilitates selective enrichment of targeted organisms. b, Relative abundance of nine-member community constituents measured by 16S rRNA sequencing before conjugative VcDART delivery and after selection for pyrF-targeted transposition in K. michiganensis or P. simiae. * indicates no growth detected in selective medium. c, Representative Sanger sequencing chromatogram of PCR product spanning transposon insertion site at targeted pyrF locus in K. michiganensis (top) and P. simiae (bottom) colonies following VcDART-mediated transposon integration and selection. Target site duplications (TSD) are indicated with dashed boxes.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A transposon system comprising:
a) a nucleotide sequence encoding polypeptides that form a CRISPR-associated transposase (CAST) complex;
b) a nucleotide sequence encoding a guide RNA comprising a nucleotide sequence that hybridizes to a target nucleotide sequence in a prokaryotic cell genome; and
c) a transposon, or an insertion site for a transposon, wherein the transposon or the transposon insertion site is flanked by recognition sites that are recognized by the CAST complex,
wherein (a) and (b) are present on the same nucleic acid construct.

2. The system of claim 1, wherein (a), (b), and (c) are all present on the same nucleic acid construct.

3. The system of claim 1, wherein the nucleic acid construct is a conjugative nucleic acid construct.

4. The system of claim 2, wherein the nucleic acid construct is a conjugative nucleic acid construct.

5. The system of claim 1, wherein the CAST complex comprises:
a) a Cas12k polypeptide, a tnsC polypeptide, a tnsB polypeptide, and a tniQ polypeptide; or
b) a Cas6 polypeptide, a Cas7 polypeptide, a Cas8 polypeptide, a tnsA polypeptide, a tnsB polypeptide, a tnsC polypeptide, and a tniQ polypeptide.

6. The system of claim 5, wherein:
a) the Cas12k polypeptide comprises an amino acid sequence having at least 50% amino acid sequence identity to the amino acid sequence depicted in any one of FIG. 4A and FIG. 6F-6J;
b) the tnsC polypeptide comprises an amino acid sequence having at least 50% amino acid sequence identity to the amino acid sequence depicted in any one of FIG. 4C and FIG. 6L-6N;
c) the tnsB polypeptide comprises an amino acid sequence having at least 50% amino acid sequence identity to the amino acid sequence depicted in any one of FIG. 4B and FIG. 6A-6E; and
d) the tniQ polypeptide comprises an amino acid sequence having at least 50% amino acid sequence identity to the amino acid sequence depicted in any one of FIG. 4D and FIG. 6O-6R.

7. The system of claim 5, wherein:
a) the Cas6 polypeptide comprises an amino acid sequence having at least 50% amino acid sequence identity to the amino acid sequence depicted in any one of FIG. 5G and FIG. 7M-7O;
b) the Cas7 polypeptide comprises an amino acid sequence having at least 50% amino acid sequence identity to the amino acid sequence depicted in any one of FIG. 5F and FIG. 7P-7R;
c) the Cas8 polypeptide comprises an amino acid sequence having at least 50% amino acid sequence identity to the amino acid sequence depicted in any one of FIG. 5E and FIG. 7S-7U;
d) the tnsA polypeptide comprises an amino acid sequence having at least 50% amino acid sequence identity to the amino acid sequence depicted in any one of FIG. 5A and FIG. 7A-7C;
e) the tnsB polypeptide comprises an amino acid sequence having at least 50% amino acid sequence identity to the amino acid sequence depicted in any one of FIG. 5B and FIG. 7D-7F;
f) the tnsC polypeptide comprises an amino acid sequence having at least 50% amino acid sequence identity to the amino acid sequence depicted in any one of FIG. 5C and FIG. 7G-7I; and
g) the tniQ polypeptide comprises an amino acid sequence having at least 50% amino acid sequence identity to the amino acid sequence depicted in any one of FIG. 5D and FIG. 7J-7L.

8. The system of any one of claims 1-7, wherein the transposon has a size of up to 100 kb.

9. The system of any one of claims 1-8, wherein the construct comprises a promoter operably linked to the nucleotide sequence encoding the CAST complex polypeptides and to the nucleotide sequence encoding the guide RNA, wherein the promoter is functional in a prokaryotic cell.

10. The system of any one of claims 1-9, wherein the construct comprises a selectable marker.

11. The system of any one of claims 1-9, wherein the construct does not comprise a selectable marker.

12. The system of any one of claims 1-11, wherein the transposon comprises one or more nucleotide sequences encoding one or more polypeptides that confer antibiotic resistance on a bacterium.

13. The system of any one of claims 1-11, wherein the transposon comprises one or more nucleotide sequences encoding one or more enzymes in a biosynthetic pathway.

14. The system of any one of claims 1-11, wherein the transposon comprises one or more nucleotide sequences encoding a polypeptide that inhibits viability and/or growth of a prokaryotic cell.

15. The system of any one of claims 1-11, wherein the transposon comprises one or more nucleotide sequences encoding one or more enzymes in a carbon utilization pathway.

16. The system of claim 15, wherein the carbon utilization pathway is a polysaccharide utilization pathway.

17. The system of any one of claims 1-16, wherein the transposon comprises one or more nucleotide sequences encoding one or more detectable markers.

18. The system of claim 17, wherein the detectable marker is a fluorescent polypeptide.

19. A prokaryotic cell comprising the system of any one of claims 1-18.

20. A library of nucleic acids comprising a plurality of member conjugative nucleic acid constructs, wherein each member conjugative nucleic acid construct comprises:
a) a nucleotide sequence encoding CRISPR-associated transposase (CAST) complex polypeptides;
b) a nucleotide sequence encoding a guide RNA comprising a nucleotide sequence that hybridizes to a target nucleotide sequence in a prokaryotic cell genome;
c) a transposon, wherein the transposon is flanked by recognition sites that are cleaved by the transposase.

21. The library of claim 20, wherein each member conjugative nucleic acid construct comprises a nucleotide sequence that provides a unique nucleotide sequence barcode that identifies the member.

22. A library of prokaryotic cells comprising the library of claim 20 or claim 21.

23. A method of editing the genome of a target prokaryotic cell, the method comprising introducing into the target bacterium the transposon system of any one of claims 1-18.

24. The method of claim 23, wherein said introducing comprises contacting one or more target prokaryotic cells with one or more prokaryotic cells according to claim 19, and wherein the construct is transmitted conjugatively from said one or more prokaryotic cells to the one or more target prokaryotic cell.

25. The method of claim 23 or claim 24, wherein the one or more target prokaryotic cells are: a) one or more prokaryotic cells present in or enriched from a natural environment; or b) one or more prokaryotic cells present in a synthetic community of prokaryotic cells.

26. The method of claim 25, wherein the one or more one target prokaryotic cells are one or more gut bacteria.

27. The method of claim 25, wherein the natural environment comprises soil.

28. The method of any one of claims 23-25, wherein the one or more target prokaryotic cells are refractory to genetic modification by electroporation and/or heat shock.

29. The method of any one of claims 23-28, wherein the target prokaryotic cells are a heterogeneous population of prokaryotic cells.

30. The method of any one of claims 23-29, wherein said introducing comprises contacting a population of target prokaryotic cells with said one or more prokaryotic cells, and wherein the method comprises, after said introducing,
identifying target cells, within the contacted population of target prokaryotic cells, that have an edited genome and/or
enriching the contacted population of target prokaryotic cells for target cells having an edited genome.

31. The method of claim 30, wherein said identifying comprises high throughput nucleic acid sequencing.

32. The method of claim 31, wherein the transposon comprises a distinguishable marker and said enriching is based on a phenotype associated with the presence or absence of the distinguishable marker.

33. The method of claim 32, wherein the distinguishable marker is a screenable marker.

34. The method of claim 33, wherein the screenable marker is a fluorescent protein encoded by the transposon.

35. The method of claim 33, wherein the screenable marker is an epitope encoded by the transposon.

36. The method of claim 33, wherein the screenable marker is a fluorescent aptamer encoded by the transposon.

37. A library of nucleic acids comprising a plurality of member nucleic acids, wherein each member nucleic acid comprises:
a) a nucleotide sequence encoding a transposon, wherein the transposon is flanked by recognition sites that are cleaved by a transposase; and
b) a nucleotide sequence that provides a unique nucleotide sequence barcode that identifies the member.

38. The library of claim 37, wherein each member nucleic acid comprises a nucleotide sequence encoding the transposase.

39. The library of claim 37, comprising a transposase bound to a member nucleic acid.

40. The library of any one of claims 37-39, wherein each member nucleic acid comprises a promoter operably linked to the transposon.

41. A method of identifying conditions for genetically modifying a prokaryotic species present in a heterogeneous population of prokaryotic cells, the method comprising:
a) contacting the heterogeneous population of prokaryotic cells with a library of nucleic acids according to any of claims 37-40 under conditions that promote introduction of nucleic acid into a prokaryotic cell, wherein said contacting generates a modified heterogeneous population of prokaryotic cells comprising genetically modified prokaryotic cells comprising the transposon inserted into the genome; and
b) identifying the species of genetically modified prokaryotic cells by sequencing the junction between the transposon and genomic DNA and/or by sequencing the nucleotide sequence barcode.

42. The method of claim 41, wherein the conditions that promote introduction of nucleic acid into a prokaryotic cell comprise conjugation, transformation, or transduction.

43. The method of claim 41, wherein the conditions that promote introduction of nucleic acid into a prokaryotic cell comprise electroporation or chemically induced competence.

44. The method of any one of claims 41-43, wherein the transposon and transposase are from a Tn5 system or a Mariner system.

45. The method of any one of claims 37-40, comprising, after step (a), amplifying the junction between the transposon and genomic DNA.

46. The method of claim 45, comprising:
a) fragmenting DNA obtained from the modified heterogeneous population of prokaryotic cells;
b) ligating adaptor DNA fragments to the fragmented DNA; and
c) amplifying the junction between the transposon and genomic DNA by polymerase chain reaction (PCR), using a forward PCR primer that hybridizes to a nucleotide sequence in the transposon and a reverse PCR primer that hybridizes to a nucleotide sequence in the adaptor DNA.

47. The method of any one of claims 41-46, wherein the heterogeneous population of prokaryotic cells comprises at least 5 different species of prokaryotic cells.

48. The method of any one of claims 41-46, wherein the heterogeneous population of prokaryotic cells comprises from 5 to 50 or from 50 to 500 different species of prokaryotic cells.

49. The method of any one of claims 41-48, wherein the heterogeneous population of prokaryotic cells is obtained from a soil sample.

50. The method of any one of claims 41-48, wherein the heterogeneous population of prokaryotic cells are from the intestinal tract of a mammal.

51. The method of any one of claims 41-48, wherein the heterogeneous population of prokaryotic cells are present in bioremediation, food, food processing, a bioreactor, an SCN bioreactor, or waste processing.


SEQUENCE LISTING (61 sequences)

SEQ ID NO: 1 - DNA, 108 bp, Artificial sequence
  synthetic sequence
tgttgatgcaaccataaagtgatatttaataattatttataatcagcaacttaaccacaaaacaaccatatattgatatctcacaaaacaaccataagttgatatttt

SEQ ID NO: 2 - DNA, 71 bp, Artificial sequence
  synthetic sequence
gcaatatcaatttatgggtgtgataattatcaatttatgggtgtaattatcattttatggttgtatcaaca

SEQ ID NO: 3 - DNA, 113 bp, Artificial sequence
  synthetic sequence
tgtacagtgacaaattatctgtcgtcggtgacagattaatgtcattgtgactatttaattgtcgtcgtgacccatcagcgttgcttaattaattgatgacaaattaaatgtca

SEQ ID NO: 4 - DNA, 211 bp, Artificial sequence
  synthetic sequence
cgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtaca

SEQ ID NO: 5 - DNA, 81 bp, Artificial sequence
  synthetic sequence
gtgaactgccgagtaggtagctgataacgagacctcgtttacctatcggtctcgtgaactgccgagtaggtagctgataac

SEQ ID NO: 6 - DNA, 254 bp, Artificial sequence
  synthetic sequence
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctaaatgggttgaaagggagaccgagatctcgaggtctcc

SEQ ID NO: 7 - DNA, 30 bp, Artificial sequence
  synthetic sequence
ctataggggatagatgtccacgaggtctct

SEQ ID NO: 8 - protein, 639 aa, Scytonema hofmanni
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRS

SEQ ID NO: 9 - protein, 584 aa, Scytonema hofmanni
MNSQQNPDLAVHPLAIPMEGLLGESATTLEKNVIATQLSEEAQVKLEVIQSLLEPCDRTTYGQKLREAAEKLNVSLRTVQRLVKNWEQDGLVGLTQTSRADKGKHRIGEFWENFITKTYKEGNKGSKRMTPKQVALRVEAKARELKDSKPPNYKTVLRVLAPILEKQQKAKSIRSPGWRGTTLSVKTREGKDLSVDYSNHVWQCDHTRVDVLLVDQHGEILSRPWLTTVIDTYSRCIMGINLGFDAPSSGVVALALRHAILPKRYGSEYKLHCEWGTYGKPEHFYTDGGKDFRSNHLSQIGAQLGFVCHLRDRPSEGGVVERPFKTLNDQLFSTLPGYTGSNVQERPEDAEKDARLTLRELEQLLVRYIVDRYNQSIDARMGDQTRFERWEAGLPTVPVPIPERDLDICLMKQSRRTVQRGGCLQFQNLMYRGEYLAGYAGETVNLRFDPRDITTILVYRQENNQEVFLTRAHAQGLETEQLALDEAEAASRRLRTAGKTISNQSLLQEVVDRDALVATKKSRKERQKLEQTVLRSAAVDESNRESLPSQIVEPDEVESTETVHSQYEDIEVWDYEQLREEYGF

SEQ ID NO: 10 - protein, 276 aa, Scytonema hofmanni
MTEAQAIAKQLGGVKPDDEWLQAEIARLKGKSIVPLQQVKTLHDWLDGKRKARKSCRVVGESRTGKTVACDAYRYRHKPQQEAGRPPTVPVVYIRPHQKCGPKDLFKKITEYLKYRVTKGTVSDFRDRTIEVLKGCGVEMLIIDEADRLKPETFADVRDIAEDLGIAVVLVGTDRLDAVIKRDEQVLERFRAHLRFGKLSGEDFKNTVEMWEQMVLKLPVSSNLKSKEMLRILTSATEGYIGRLDEILREAAIRSLSRGLKKIDKAVLQEVAKEYK

SEQ ID NO: 11 - protein, 167 aa, Scytonema hofmanni
MIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKV

SEQ ID NO: 12 - protein, 222 aa, Vibrio cholerae
MATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLES

SEQ ID NO: 13 - protein, 603 aa, Vibrio cholerae
MAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 14 - protein, 330 aa, Vibrio cholerae
MSETREARISRAKRAFVSTPSVRKILSYMDRCRDLSDLESEPTCMMVYGASGVGKTTVIKKYLNQNRRESEAGGDIIPVLHIELPDNAKPVDAARELLVEMGDPLALYETDLARLTKRLTELIPAVGVKLIIIDEFQHLVEERSNRVLTQVGNWLKMILNKTKCPIVIFGMPYSKVVLQANSQLHGRFSIQVELRPFSYQGGRGVFKTFLEYLDKALPFEKQAGLANESLQKKLYAFSQGNMRSLRNLIYQASIEAIDNQHETITEEDFVFASKLTSGDKPNSWKNPFEEGVEVTEDMLRPPPKDIGWEDYLRHSTPRVSKPGRNKNFFE

SEQ ID NO: 15 - protein, 394 aa, Vibrio cholerae
MFLQRPKPYSDESLESFFIRVANKNGYGDVHRFLEATKRFLQDIDHNGYQTFPTDITRINPYSAKNSSSARTASFLKLAQLTFNEPPELLGLAINRTNMKYSPSTSAVVRGAEVFPRSLLRTHSIPCCPLCLRENGYASYLWHFQGYEYCHSHNVPLITTCSCGKEFDYRVSGLKGICCKCKEPITLTSRENGHEAACTVSNWLAGHESKPLPNLPKSYRWGLVHWWMGIKDSEFDHFSFVQFFSNWPRSFHSIIEDEVEFNLEHAVVSTSELRLKDLLGRLFFGSIRLPERNLQHNIILGELLCYLENRLWQDKGLIANLKMNALEATVMLNCSLDQIASMVEQRILKPNRKSKPNSPLDVTDYLFHFGDIFCLWLAEFQSDEFNRSFYVSRW

SEQ ID NO: 16 - protein, 640 aa, Vibrio cholerae
MQTLKELIASNPDDLTTELKRAFRPLTPHIAIDGNELDALTILVNLTDKTDDQKDLLDRAKCKQKLRDEKWWASCINCVNYRQSHNPKFPDIRSEGVIRTQALGELPSFLLSSSKIPPYHWSYSHDSKYVNKSAFLTNEFCWDGEISCLGELLKDADHPLWNTLKKLGCSQKTCKAMAKQLADITLTTINVTLAPNYLTQISLPDSDTSYISLSPVASLSMQSHFHQRLQDENRHSAITRFSRTTNMGVTAMTCGGAFRMLKSGAKFSSPPHHRLNSKRSWLTSEHVQSLKQYQRLNKSLIPENSRIALRRKYKIELQNMVRSWFAMQDHTLDSNILIQHLNHDLSYLGATKRFAYDPAMTKLFTELLKRELSNSINNGEQHTNGSFLVLPNIRVCGATALSSPVTVGIPSLTAFFGFVHAFERNINRTTSSFRVESFAICVHQLHVEKRGLTAEFVEKGDGTISAPATRDDWQCDVVFSLILNTNFAQHIDQDTLVTSLPKRLARGSAKIAIDDFKHINSFSTLETAIESLPIEAGRWLSLYAQSNNNLSDLLAAMTEDHQLMASCVGYHLLEEPKDKPNSLRGYKHAIAECIIGLINSITFSSETDPNTIFWSLKNYQNYLVVQPRSINDETTDKSSL

SEQ ID NO: 17 - protein, 352 aa, Vibrio cholerae
MKLPTNLAYERSIDPSDVCFFVVWPDDRKTPLTYNSRTLLGQMEAASLAYDVSGQPIKSATAEALAQGNPHQVDFCHVPYGASHIECSFSVSFSSELRQPYKCNSSKVKQTLVQLVELYETKIGWTELATRYLMNICNGKWLWKNTRKAYCWNIVLTPWPWNGEKVGFEDIRTNYTSRQDFKNNKNWSAIVEMIKTAFSSTDGLAIFEVRATLHLPTNAMVRPSQVFTEKESGSKSKSKTQNSRVFQSTTIDGERSPILGAFKTGAAIATIDDWYPEATEPLRVGRFGVHREDVTCYRHPSTGKDFFSILQQAEHYIEVLSANKTPAQETINDMHFLMANLIKGGMFQHKGD

SEQ ID NO: 18 - protein, 199 aa, Vibrio cholerae
VKWYYKTITFLPELCNNESLAAKCLRVLHGFNYQYETRNIGVSFPLWCDATVGKKISFVSKNKIELDLLLKQHYFVQMEQLQYFHISNTVLVPEDCTYVSFRRCQSIDKLTAAGLARKIRRLEKRALSRGEQFDPSSFAQKEHTAIAHYHSLGESSKQTNRNFRLNIRMLSEQPREGNSIFSSYGLSNSENSFQPVPLI

SEQ ID NO: 19 - protein, 584 aa, Nostoc punctiforme
MNSQQNPDLAVHPSAIPIEGLLGESDITLEKNVIATQLSEEAQLKLEVIQSLLEPCDRTTYGQKLREAAEKLGVSLRTVQRLVKNWEHDGLVGLTQTGRADKGKHRIGEFWEKFITKTYNEGNKGSKRMTPKQVALRVEAKARELKDSKPPNYKTVLRVLAPILEKQEKAKSIRSPGWRGTTLSVKTREGKDLSVDYSNHVWQCDHTRVDVLLVDQHGELLSRPWLTTVIDTYSRCIMGINLGFDAPSSGVVALALRHAILPKQYGFEYKLHCEWGTYGKPEHFYTDGGKDFRSNHLSQIGAQLGFVCHLRDRPSEGGVVERPFKTLNDQLFSTLPGYTGSNVQERPKDAEKDARLTLRELEQLLIRYIVDRYNQSIDARMGDQTRFERWEAGLPTVPVPIPERDLDICLMKQSRRTVQRGGCLQFQNLMYRGEYLAGYAGETVNLRFDPRDITTILVYRQENNQEVFLTRAHAQGLETEQLALDEAEAASRRLRNAGKTISNQSLLQEVVDRDALVATKKSRKERQKLEQAVLRSAGVDESKTESLSSQVVEPDEVESTETVHSQYEDMEVWDYEQLREEYGF

SEQ ID NO: 20 - protein, 569 aa, Anabaena cylindrica
MYMRNETPLTPGNLGDESSIGKETQVLVSELSGEAKLKMEVIQSLLEAGDRTTYAQRLKEAAVKLGKSVRTVRRLIDKWEEEGLVGLTQTERVDKGKHRVDENWQEFILKTYKEGNKGGKRMSRQQVAVRVKVRADDLGVKPPSHMTVYRILEPVIEKQEKAKSIRSPGWRGSRLSVKTRDGKDLQVEYSNQVWQCDHTRADVLLVDKHGEILGRPWLTTVIDSYSRCIVGINLGYDAPSSQVVALALRHAILPKQYGQEYELYEEWGTYGKPEHFYTDGGKDFRSNHLQQIGVQLGFACHLRDRPSEGGIVERPFKTFNTQLFSTLQGYTGSNVQERPEEAEKEACLTLRELEQRLVAYIVNTYNQREDARMGDQTRFQRWDSGLIVAPEVISERDLDICLMKQSRRMIQRGGYLQFENLMYKGEHLAGYAGESVVLRYDPRDITTILVYSHKGHKEEFLARAYAQDLETEELSLDEAKAMIRRIREAGKTISNRSMLAEVRDRETFVKQKKTKKERQKEEQAVVQKTKKPVPVEPVEEIEVAFVESSQETDMPEVFDYEQMREDYGW

SEQ ID NO: 21 - protein, 584 aa, Hassallia byssoidea
MNSQQNPDLAVHTSAIPTEGLQEESDKPLERNVIVTALSEEAQLKLEVIQSLLEKSDRTTYGQKLKEAAEKLSVSVRTVQRLVKKWEQDGVVGFTQTGRADKGRHRIGEFWENFILKTYREGNKGSKRMTPKQVALRVQAKARELEDSKPPNYRTVLRVLAPVLEKQEKAKSIRSPGWRGTTLSVKTREGKDLSVDYSNHVWQCDHTRVDVLLVDQHGQLLSRPWLTTVIDTYSRCIMGINLGFDAPSSVVVALALRHAILPKQYGSEYKLHCEWGTYGKPEHFYTDGGKDFRSNHLSQIGAQLGFVCHLRDRPSEGGVVERPFKTLNDQLFSTLPGYTGSNVQERPEDAEKDARLTLRELEQLLVRYLVDRYNQSIDARMGDQTRFERWEAGLPTVPVPIPERDLDICLMKQSRRTVQRGGCLQFQNLMYQGEYLAGYAGETVNLRFDPRDITTILVYRQENNQEVFLTRAHAQGLETEQLALDEAEAASRRLRTAGKTISNQSLLQEVVDRDALVATKKSRKERQKLEQAVLRSAGVDESKTESLSSQVVEPDEVESTAEINSQYEDMEVWDYEQLREEYGF

SEQ ID NO: 22 - protein, 582 aa, Anabaena sp.
MNRDENSDLNTSAIPVESISEGDNTPPETNVIATELSEESQLKLEVLQSLLEPCDRTTYGQKLKEAAEKLAVSVRTVQRLVKKWEQDGLVGLTQTGRTDKGKHRIGEFWEDFIVKTHKEGNKGSKRMSPKQVAIRVQAKAHELSDLKPPNYRTVLRVLAPILEKQEKTKSIRSPGWRGTTLSVKTREGKDLSVDYSNHVWQCDHTPADVLLVDQHGELLSRPWLTTVIDTYSRCIMGINLGFDAPSSEVVALALRHAILPKRYSLEYKLHCEWGTYGKPEHFYTDGGKDFRSNHLSQIGAQLGFVCHLRDRPSEGGIVERPFKTLNDQLFSTLPGYTGSNVQERPEDAEKDAKLTLRELEQLIVRYIVDRYNQSIDARMGDQTRFGRWEAGLPSVPVPIEERDLDICLMKQSRRRVQKGGHLQFQNLMYRGEYLGGYDGETVNLRFNPRDITTVLVYRQENSQEVFLTRAHAQGLETEQLSLDEAEAASRRLRNLGKTISNQALLQEVLDRDALVANKKSRKDRQKLEQEILRSTAVNDSKNESLASPVMEAEDVEFTTPVQSSSPELEVWDYEQLREEYGF

SEQ ID NO: 23 - protein, 558 aa, Rippkaea orientalis
MNTFPNEQSNAIVLKNTIVSDLPETARAKMEVIQTLLEPCDRTTYGERLREGAKKLGVSVRTVQRLFKQYQEQGLAALVSMERADKGKHRINEFWQDFIVKTYQQGNKGSKRMTPKQVALKVQAKALEIGDEQPPTYRTVLRVLKPIQEKQEKTKSIRSPGWRGSTLSVKTRDGDDLEINYSNQVWQCDHTRADVLLVDRHGELIGRPWLTTVIDSYSRCIMGINLGFDAPSSQVVALALRHAILPKRYGDEYKLHCEWETSGTPEYFYTDGGKDFRSNHLAQIGSQLGFVHKLRDRPSEGGIVERPFKTLNQSLFSTLPGYTGSNVQERPKEAEKEASLTLRELEQLIVRFIVDKYNQSIDARMGDQTRYQRWEAGLRRQPEPISERELDICLMKAARRTVQRGGYLQFENVMYRGEYLEGYAGDTVILRYEPRDITTIWVYRQKKHQEVFLTRAHAQDLETEQLSVDEAKASAKKLRDAGKTISNQSILQEVIEREALVEKKSRKQRQKAEQAYKQEKQPSIIETVEPIEPEPLTQTEVDDIEVWDYDQLRDDYGW

SEQ ID NO: 24 - protein, 639 aa, Hassallia byssoidea
MSQITIQCCLIASESTRQKLWKLMAHLNTPLINELLQQLSKHPDFEKWRKNGKLPSTVVNQLCQPLKTDPSFTGQPSRLYLSAIHVVDYIYKSWLAIQKRLQQQLDGKIRWLEMLNSDAELIEISGCSLEAIRTKAAEILAIATPDSDVAAPLTKTGKAKKSKKSSASNPDRSLSHKLFDAYQETDDILSRSAISYLLRNGCKLNDKEEDLEKFAKRRRKVEIQIQRLTDKLTSRIPKGRDLTNAKWLETLFTATTTVPEDNVEAKRWQDILLTRSSSVPFPLIFETNEDLVWSKNEKGRLCVHFNGLSDLTFEVYCDRRQLHWFKRFLEDQQTKRKSKNQHSSGLFTLRNGRLAWQEGEGKGEPWQINRLTLYCCVDNRLWSAEGTEQVRQEKEEEITKFITKMNEKSDLSETQQAFIKRKESTLTRINNSFERPSQFLYQGQSHILVGVSLGLEKPATVAVVDAIAGKVLAYRSIKQLLGDNYELLNRQRRQQQYLSHERHKAQKSFSPNQFGTSELGQYVDRLLAKEIIAIAQTHKAGSIVLPKLGDMREIVQSEIQAIAEEKFPGYVEGQQKYAKQYRVNVHGWSHSRLIQSIQSKAAQIGIVIEEGKQPIRGSPQDKAKELALSAYNLRLARRS

SEQ ID NO: 25 - protein, 639 aa, Nostoc punctiforme
MSQITIQCRLIASESTRQKLWKLMATLNTPLINELIEQLGKHPDFENWRQQGKLPTTVVSQLCQPLKTDPRFVGQPSRLYMSAIHIVDYIYKSWLAIQKRLQQQLDGKMRWLEMLNSDVELVETSGSSMGAIRTKASEILAKAMPTSDSDSSQPKTKKGKEAKKSSSSSSDRSLSNKLFEAYQETEDILSRSAISYLLKNGCKLSDKEEDSEKFAKRRRQVEIQIQRLTEKLISRMPKGRDLTNRKWLETLFTATTTFPEDNAEAKRWQDILLTRPSSLPFPLVFETNEDMVWSKNQKGRLCVHFNGLSDLSFEVYCDNRQLHWFQRFLEDQQTKRQSKSQYSSGLFTLRNGHLVWQEGEGKSEPWNLNRLNLYCCVDNRLWTADGTEQVRQEKAEEISKLITKMKEKSDLKDTQKAFIQRKESTLNRMNNSFERPSQPLYQGQSHILVGVSLGLEKPATVAVVDAIAGKVLAYRSIRQLLGDNYELLNRQRRQQRSSSHERHKAQKSFSPNQFGTSELGQYVDRLLAKEIIAIAQTYKAGNIVLPKLGDMREIVQSEIQAIAEAKCPGSVEVQQKYAKQYRVNVHKWSYGRLIQSIQSKGSQAGIVIEEGKQPVRGSPHEQAKELALSAYHDRLARRS

SEQ ID NO: 26 - protein, 639 aa, Anabaena sp.
MSQITVQCRLIASESTRQQLWTLMAELNTPLINELLQQLSKHPDFEKWRKDGKFPSTVVSQLCQPLKTDPQFAGQPSRCYLSAIHVVDYIYKSWLTIQKRLQQQLDGKIRWLEMLNSDAELVETSGYSLEAIRTKAAEILAMTTPESDTNVPLTKKRNTKKSKKSSASNPEPSLSHKLFNAYQETDDILSRSAISYLLKNGCKLNDKEEDTEKFAKRRRKVEIQIQRLTDKLTSRIPKGRDLTNSKWLETLFTAITTVPEDNAEAKRWQDILSTRSSSLPFPLIFETNEDLKWSTNEKGRLCVHFNGLTDLTFEVYCDSRQLHWFKRFLEDQQTKRKSKNQHSSGLFTLRNGRLAWQEGEGKGETWQIHRLTLSCCVDNRLWTAEGTEQVRQEKAEDITKFITKMKEKSDLSDTQQAFIQRKQSTLTRINNSFDRPCKPLYQGQSHILVGVSMGLEKPATVAVVDASANKVLTYRSIKQILGENYELLNRQRRQQRSSSHERHKAQKSFSPNQFGTSELGQYIDRLLAKEIVAIAQTYKAGSIVLPKLGDMRENIQSEIQAIAEIKCPGSVEIQQKYAKQYRINVHKWSYGRLIQSIQSKAAQVGIVIEEGKQPVRDSPQDKAKELALSTYHLRLAKQS

SEQ ID NO: 27 - protein, 635 aa, Anabaena cylindrica
MSQITIQCRLVASETTRQQLWQLMAEKNTPLINELLSQIGKHPEFETWRQKGKHPTGIVKELCEPLKTDPRFIGQPARFYTSATASVNYIYESWFALMKRYQSQLDGKLRWLEMFNSDAELVEHSGVSLDTLRATSAEILAQFAPQDTNRDTSNKGKKSKMGKKSQKSDSEGNLSKKLFDAYSSAEDNLTRCAISHLLKNGCKVSNKEENSEKFTQRRRKLEIQIQRLTEKLAARIPKGRDLTDTQWLETLFTATYNVPEDETEAKLWQNSLLRKFSSLPFPVAYETNEDLVWSKNRFGRICLTFPTLREHIFQIYCDSRQLHWFQRFLEDQEIKKNSKNQHSSALFTLRSGRIAWQEGEGKGEPWDIHHLTLYCCVDTRLWTEEGTNLVKEEKAEEIAKTITQTKAKGDLNDKQQAHLKRKNSSLARINNPFPRPSQPLYKGQSHILLGVSLGLEKPATVAVVDGTTGKVLTYRNIKQLLGDNYKLLNRQRQQKHLLSHQRHIAQRIAAPNNFGDSELGEYIDRLLAKEIIAIAQTYQAGSIVLPNLGDMREQIQSEIKAKAEQKSDLVEVQKKYAKQYPNSVHQWSYGRLITNIQSQSKKAGIVIEEGKQQIRASPLEKAKELAINAYQSRKA

SEQ ID NO: 28 - protein, 642 aa, Anabaena cylindrica
MSVITIQCRLVAEEDSLRQLWELMSEKNTPFINEILLQIGKHPEFETWLEKGRIPAELLKTLGNSLKTQEPFTGQPGRFYTSAITLVDYLYKSWFALQKRRKQQIEGKQRWLKMLKSDQELEQESQSSLEVIRNKATELFSKFTPQSDSEALRRNQNDKQKKVKKTKKSTKPKTSSIFKIFLSTYEEAEEPLTRCALAYLLKNNCQISELDENPEEFTRNKRRKEIEIERLKDQLQSRIPKGRDLTGEEWLETLEIATFNVPQNENEAKAWQAALLRKTANVPFPVAYESNEDMTWLKNDKNRLFVRFNGLGKLTFEIYCDKRHLHYFQRFLEDQEILRNSKRQHSSSLFTLRSGRIAWLPGEEKGEHWKVNQLNFYCSLDTRMLTTEGTQQVVEEKVTAITEILNKTKQKDDLNDKQQAFITRQQSTLARINNPFPRPSKPNYQGKSSILIGVSFGLEKPVTVAVVDVVKNKVIAYRSVKQLLGENYNLLNRQRQQQQRLSHERHKAQKQNAPNSFGESELGQYVDRLLADAIIAIAKKYQAGSIVLPKLRDMREQISSEIQSRAENQCPGYKEGQQKYAKEYRINVHRWSYGRLIESIKSQAAQAGIAIETGKQSIRGSPQEKARDLAVFTYQERQAALI

SEQ ID NO: 29 - protein, 276 aa, Nostoc punctiforme
MTEAQAIAKQLGGVKPDDEWLQAEIARLKGKSIVPLQQVKTLHDWLDGKRKARQSCRVVGESRTGKTVACDAYRYRQKPQQEVGRPPIVPVVYIQPPQKCGSKDLFKEMIEYLKFRATKGTVSDFRGRAMEVLKGCGVEMLIIDEADRLKPETFAEVRDIYDKLGIAVVLVGTDRLEAVIKRDEQVYNRFRACHRFGKLSGKDFQDTVQAWEDRVLKLPVSSNLTSKDMLRILTSATEGYIGRLDEILRETAIRSLSKGFKKIDKAVLQEVAKEYK

SEQ ID NO: 30 - protein, 276 aa, Chlorogloea sp.
MIEAQDFANQLGGVKPDDEWLQIEITRLKGKSIVPLHQVKTLHDWLDGKRKSRQSCRVVGESRTGKTVACDAYRYRQKPQQEVGRPPIVPVVYIQPPQKCGSKDLFKEMIEYLKFRATNGTVSDFRGRAMEVLKGCGVEMLIIDEADRLKPETFAEVRDIYDKLGIAVVLVGTDRLEAVIKRDEQVYNRFRACHRFGKLSGKEFQDTVQAWEERVLKLPLSSNLTSKDMLRILTSATGGYIGRLDEILRDAAIRSLSKGFKKIDKAVLQAVAQEYK

SEQ ID NO: 31 - protein, 276 aa, Leptolyngbya frigida
MVQAKELAEVLGDFKPTEDQSKTEIDRLRKKTIMPLEQVSRLHSWLDGKRQSRQSCRVVGESRTGKSVACAAYLHRHKPQQAAGKKPIVPVVYIQPPQKCGAKDLFKAIIEFLKHRATKGTISDFRGRAMEVLQACEVEILIIDEADRLKPETFADVRDIYDKLAISVVLVGTDRLDAVIKCDEQVHNRFRACYRFGKLSGEEFAKTVALWEQQILKLPVASNLTSKEMLRLLTAGTEGYIGRMDEILREAAIRSLSAGFKKVDKKILEEVAKEYK

SEQ ID NO: 32 - protein, 278 aa, Anabaena sp.
MTDDAQAIAKQLGGVKPDEEWLQAEITHLTSKSIVPLQQVITLHDWLDGKRKARQSCRVVGESRTGKTVACDAYRYRHKPRQEMGKTPIVPVVYIQPPSKCGAKDLFQEIIEYLKFKATRGTISDFRGRTMEVLKGCRVEMIIIDEADRIKPDTFADVRDIYDKLGIAIVLVGTDRLEAVIKRDEQVYNRFRACHRFGKLAGKDFQDTVQAWEDKILKLPLPSNLISKDMLRILTSATEGYIGGLDEILREAAIRSLSRGLKKIDKAVLQEVVQEFKL

SEQ ID NO: 33 - protein, 167 aa, Nostoc punctiforme
MMTTPDVKPWLFIIEPYPGESLSHFLGRFRRANHLSPAGLGGLAGIGAVVARWERFHFNPRPSQKELEAIASVVEVDAQRLAQMLPPAGVGMQHEPIRLCGACYAEAPCHRIEWQYKSVWKCDRHQLKILAKCPNCQAPFKMPALWEDGCCHRCRTLFAEMAKQQKS

SEQ ID NO: 34 - protein, 167 aa, Aphanixomenon flos-aquae
MTAPDVKPWLFIIQPYEGESLSHFLGRFRRANHLSAAGLGNLAGIGAVIARWERFHFNPRPSQKELEAVASVVEVDAQRLVQMLPPVGVGMQHEPIRLCGACYAETPCHQIKWQFKETGGCDRHNLRLLSKCPNCDARFKIPALWELGVCQRCLMTFTEMAESQKPV

SEQ ID NO: 35 - protein, 168 aa, Chlorogloea sp.
MTAPDIKPWLFIVEPYLGESLSHFLGRFRRANHLSPAGLGNLAEIGAVVARWERFHFNPRPSQQELSAIASVVEVDAKTLAEMLPPAGVGMQHEPVRLCGGCYAEIPCHRIAWQFKETGKCDRHNLRLLSKCPKCEARFKIPALWEDGCCNRCRLPFGNMVSCQKLAL

SEQ ID NO: 36 - protein, 167 aa, Leptolyngbya frigida
MGEVDIQPWLFQIEPYEGESFSHFLGRFRRPNHLTPSGLGQLAGIGAVVARWERFHLNPFPAPQELELLAIVVGVSAERLAGMLPPKGIGMQWTPLRLCGACYGEMPCHRMEWQYKSVWKCDRHQLKLLCKCAKCEAPFKMPALWEDGNCHRCLTPFAAMALHQKAA

SEQ ID NO: 37 - protein, 229 aa, Vibrio vulnificus
MTSLPTPSAITTLALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESPFELASYG

SEQ ID NO: 38 - protein, 229 aa, Vibrio diabolicus
MSSLRLPSAITISALEQAFDTPARNLTKSRGKNIHRFTSAKMGKRISVESTLECDACYHFDFEPSIVRFCAQPIRLSYFINGKHHTYVPDFLVQFDTHEFVLYEVKSDYAKNKSDFGSEWEAKVQAASALGVELELVGESDIRDKVILKNLKLMHRYASRADLNNVQKSLLSTLKQDGAQTAKCLGEQLGLKGRTILPILCDLLSRCLLDTRLDMPLSLESQFELGSYA

SEQ ID NO: 39 - protein, 229 aa, Photobacterium leiognathi
MSCLPTLSTTTISALENAFDTPARNLKKSRGKNIHRFASAKMGKRITVESSLECDACYHFDFEASIVRFCSQPIRLIYFINGKRHTYVPDFLVQFDTHEFVLYEVKSDYAKNKPDFDFEWEAKVQAAFELGLELELLEERDIRDKVILKNLKLMHRYASRDDLDNAQNALLSLLKNNGTQTARSLGEHLGLKGRTILPILCNLLSRCLLYTCLNKPLSLESQFKLGSYA

SEQ ID NO: 40 - protein, 603 aa, Vibrio chagasii
MTKKSFSSFHRKSVLHQEKLEQNDRVVDINDVAEATYKDISAFPEKIVVEITFRLSILRLLGRKCEKIVPKSIEPHRVDLQRSHDRKIPSAITIYRWWLTFRESDYNPVSLAPDFKSRGNRDPKVAPIVDAIMKQAVESVISGRKININSAYRRVKRKVRQYNLTHSTKYKYPEYESVRIRVKKKTPFEILAAKKGERVAKREFRRMGRKILTSSVLERVEIDHTVLDLFAVHEEHRIPLGRPWLTQLVDCYSKAVIGFYLGFEPPSYMSVSLALKNAIQRKDTLLSSYPSIENEWLCYGIPDLLVTDNGKEFLSKAFDKACESLLINVHQNKVETPDNKPHVERNYGTINTSLLDDLPGKAFSQYLQREGYDSVSEATLTLDEIKEIYLIWLVDIYHRKPNQRGTNCPNVAWRQGCQNWEPEEFLGSKDELDFKFAIEDHKQLTKAGITVSKGLTYSSERLAGYMGKKGNHKVQFKYNPECMAVIWVLDEDVNEYFTVNAIDYESARRVSLWQHKYNMKYQAELNSAEYDEDKEIDAEIKIEEIADRSILETKKIRSRRRGARHQENSARAKSISNTKLVPPQKDEEEIVIVDNEDWDIDYV

SEQ ID NO: 41 - protein, 598 aa, Vibrio sp.
MPKKSFSSFHRKSALQQEKLEQNDRVVDSNDVDEATYQDISAFPENIANQITFRLSILRFLAGKCEKITPKTIEPYRVALQQLHDKNIPSAISIYRWWLVFRASGYNPVSLAPDFKSRGNRGAKVSPIVDSIIEQAVERVISGRKINISSAHRRVKRKVRQYNLTHGTKYPYPKYESVRIRVKKKTPFEVLAAKKGERVAKREFRRMGKKITTSAVLERVEIDHTMVDIFAVHPVHRVTLGRPWLTQLVDCYSKAVIGFYLGFEPPSYVSVSLALKNAIQRKDDLLSSYESIENEWLCYGIPDLLVTDNGKEFLSKAFDKACESLLINVHQNRVEVPDNKPDVERKYGTINTSLLDDLPGKAFSQYLQREGYDSVGEATLTLDEIQEIYLIWLVDIYHKDSNQRGTNCPNIAWRNGCQEWEPEEFSGSKDELDFKFAIVDSKQLTKAGVTVYKGLTYSSERLAGYRGKKGNHKVQFKYNTECMAVIWVLDEDVNEYFTVNAIDYEYASGVSLWQHKYNIKYLAEYDEDKEIDAEIKIEEIADRSILETKKIRSRRRGARHQENSARAKSISNTKLVPPQKDEEEIVIVDNEDWDIGYV

SEQ ID NO: 42 - protein, 608 aa, Vibrio parahaemolyticus
MPKKSFSDFNRKAKLDVAAHQKEAIDIDRSPSEVLAEDLTYTDLTAFPDKVANEISYRLKVLRYLGKVCDNITPKTIEPHRVELPKLNDKKIPSAITIYRWWLKFSQSDYNPICLAPDLKGRGNRKSKVPKVVDAVMEQAVEGVISGRKINISSAHRRVRRKVRQYNLKHGTKYKYPKYESLRKRVKKKTPFEVLVAKKGERVAKREFRRMGKKILTSYALERVEVDHTVLDLFVVHEEYRIPLGRPYLTQLVDCYSKAVVGFYLGFEPPSYVSVSLALKNAIQRKELLLSSYPSVKNEWLCYGIPDLLVTDNGKEFLSKAFDAACETLLITVHQNKVETPDNKPHVERNYGTINTTVLDDLPGKAFSHYLHREGYDSIGEATLTLDEIKEIYLIWLVDIYHKHPNQRGTNCPNVAWKRGCEEWEPEEFTGTEAELDFKFAILDKKKLSKSGITVYVDLTYSSERLAEYRGCKGNHMVTFKYNPECMGAIWVLDEDTNEYITVPAINYEYASGVSLWQHKANIKYEKNLNFSEYDEDAEVDAEIRMEEIAEESIVKTKKMRNRRRGARYQENGERAKSHNQRLLEKTEQGHPQEEIIDDENAWGIDYL

SEQ ID NO: 43 - protein, 330 aa, Vibrio fluvialis
MNETREARISKAKRAFVSTPSVTKILCYMDRCRDLSDFESEPTCMMVYGASGVGKTTIIRKYLSQNKRDSEVGGDIIPVLHIELPDNAKPVDAARELLVEMGDPLALYETDLARLTKKLIDLIPLVGVKLIIIDEFQHLVEERSNRVLTQVGNWLKMILNKTKCPIVLFGMPYSKVVLQANSQLHGRFSIQFELRPFSYQGGNGVFKTFLEYLDKALPFEKQAGLANESLQKKLYAFSQGNMRSLRNLIYQASIEAIDNQHASITEEDFVFASKLTSGDKPISWKNPFDEGVKVTEDMLRPPPKDIGWEDYLRNTTHKIRNSGVKNNFFD

SEQ ID NO: 44 - protein, 329 aa, Vibrio splendidus
MDEDRETRISKAKRAFVSTPSVTKILGYMDRCRELSDFESEPTCMMVFGASGVGKTTIIKKYLSQNKRDSEARGDVVPVLHIELPDNAKPVDAARELLLEMRDPLALYETDLARLTKRLTDLIPVIGVKLIIIDEFQHLVEERSNRVLTQVANWIKMILNKTKCPIVLFGMPYSKVVLKANSQLHGRFSIQFELRPFSYQNGEGVFKTFLEHLDKALPFEKEVGLVEQGLQKKLYAFSQGNMRSLRNLIYQASVEAIDKQHETITEQDLIFASKLTSGDKSDRWENPFEKGVKVTEGMLRSPPKDIGWEDYYHHVTSLNAKRNGGNMFE

SEQ ID NO: 45 - protein, 330 aa, Vibrio diazotrophicus
MDDSREIRIARAKKAFVITPSVAKVLRYMDRCRDFSDMDSEPTCMIVYGASGVGKTTIIKKYLKKNEGDSDIYRDTIPVVHIELPDNAKPVDAARELLLNMGDPLALFDKDLARLTKRIVELIPALGVKLIIIDEFQHLVEESSNKILTQVGNWLKGILNKSKCPIVLFGMPYSKLVLQANSQLQSRFSIQFDLRPFSYQEGEGTFKTFLQHLDEALPFEKQAGLAKEGLQKKLYAFSQGNMRSLRDLIYQASIEAIDNHHESITKDDFLFSSQLTSGNKPTFWKNPFIEGVKVTKDMLRSPPSSIGWEDYYQQNNSRKKKGKGRPDFFD

SEQ ID NO: 46 - protein, 394 aa, Vibrio vulnificus
MFLQRPKPYSDESLESFFIRVANKNGYDDVHRFLEATKRFLQDIDHNGYQTFPTDITRINPYSAKNSSSARTASFLKLAQLTFNEPPELLGLAINRTNMKYSPSTSAVVRGAEVFPRCLLRTHSIPCCPLCLRENGYASYLWHFQGYEYCHTHNVPLITTCSCGKEFDYRVSGLKGVCCKCKEPITLTSRENGHEAACTVSNWLAGHESKPLPNLPKSYRWGLVHWWMGIKDSEFDHFSFVQFFSNWPRSFHSIIEDEIEFNLEHAVVSTSELRLKDLLGRLFFGSIRLPERNLQHNIVLGELLCYLEKRLWQDKGLIANLKMNALETTVMLNCNLDQIASMVEQRILKPNRRTKPNSPLDVTAYLFHFGDIFCLWLAEFQTDEFKRSFYVSRW

SEQ ID NO: 47 - protein, 394 aa, Vibrio anguillarum
MFLQRPKPYSDESLESFFIRVANKNGYDDVHRFLEATKRFLQDIDHHGYQTFPTDITRINPCSANNSSRARTASLLKLAQLTFNEPPELLGLALNRTNLQYSPSTSAVIRGAEVLPRSLLRTNSISSCPLCLQENGYASYLWHFKGYDHCHIHNIPLINACSCGAEFDYRVCGLKGICNNCKEPITTKNQENSYEATSTVSNWLAGNGSQDLPDIPRSYRWGLIHWWMNLNDNEFDHLSFTHFFSNWPRSFHSMIDDEIEFNLEHAVVSTSELRLKDLLGRLFFHSIRLPERNLQHNIILGELLSHLEKRLWRDKGLIANLKMNALEASVMLNCSLEQIASMVEQRILKPNRRTKPNSPIETTDYLFHFGDIFCLWLAEFQTDEFNRSFYVSRW

SEQ ID NO: 48 - protein, 394 aa, Vibrio campbelli
MFLQRPKPYSDESLESFFIRVANKNGYSDVHRFLEATKRFLQDIDHTGYQTFPTDIAKINPCSAKDSSGARTASLLKLAQLTFNEPTELLGLAINRTNLKYSPSTSAVIRGSEVFPRSLLRTRSIPCCPLCLQENGYASYLWHLEGYDHCHIHNAPLITSCSCGNEFDYRVSELKGVCSNCKEPIATKSRGREPKQAYNVANWLAGNGSEELPNLPKSYRWGLIHWWTHLNDDKFDHGSFTHFFANWPSAFHSMIDAEIEFNLEHAFVNISELRLKDLLGRLFFSSIRLPERNLRHNIILSELLSHLEKRLWQDKGLIANLKMNALEATVLLNCSLDQIASIVAQRILKPNRRTKPNSPLEHTDYLFHFGDIFCLWLAEFQTDEFNRSFYVSRW

SEQ ID NO: 49 - protein, 199 aa, Vibrio anguillarum
MKWYYRTITFLPERCNNETLAAKCLRVLHGFNYQYETRNIGVSFPLWCDATVGKKISFVSKNKIELDLLLKQHYFVQMEQLQYFNISNTVLVPEDCKYVSFKRCQSIDKVTPAGQARKIRRLQKRALSRGESFDPSSFAQKEHTVIAHYHSLGESSKQTNRSFRLNIRMISEQHREGNSVFSSYGLSNSENSFQPVPLI

SEQ ID NO: 50 - protein, 199 aa, Vibrio parahaemolyticus
MKWYYRTITFLPERCNNETLAAKCLHVLHRFNYRYETRNIGVSFPLWCDATVGKKISFVSKNKIELDLLLKQHYFIQMEQLQYFHISNTALIPEDCTYVSFRRCQSIDKVTAAGQARKIRRLEKRALSRGESFDLSSFAQKEHTVIVHYHSLGESSQQTNHNFRLNIRMLSEQHREGNSVFSSYGLSNSENSLQPVPLI

SEQ ID NO: 51 - protein, 199 aa, Vibrio mediterranei
MKWYYRTITFLPERCNNESLAAKCLRVLHGFNYRYETRNTGVSFPLWCDETVGKKVSFISTNKMELDLLFKQHYFVQMEKLQYFSISKTVLVPEGCRYVSFKRCQSIDKVTTAGHERKIRRLEKRAKSRGEPFDPTSFTQKEHTVLSHYHSLEESSSKTNNNFRLNVRMFSEQHNEGGSVFSSYGLSNSEHSLQPVPLI

SEQ ID NO: 52 - protein, 352 aa, Vibrio cidicii
MKLPTNLAYERSIDPSDVCFFVVWPDNKKTPLTYTSRTLLGQMETASLAYDSSGTPKESATAEALAQGNPHQVDFCHVPFGASHVECCFSVSFYSELRKPYKCNSSKIKQTLVQLVELYETKIGWTELATRYLMNICNGKWLWKNTRKAYCWNIALTPWPWSGEKVEFEDIRTNYTSRQDFENNKNWSAIVEMIKTAFSSTDGLAIFEVRATLHLPTNAMVRPSQVFTEKESGSKSKSKTQNSRVFQSTTIDGERSPILGAFKTGAAIATIDDWYPDATEPLRVGRFGVHHEDVTCYRHPSTGKDFFSILQQAEHYIEVLSANKAADQDTINDMHFLMANLIKGGMFQHKGD

SEQ ID NO: 53 - protein, 352 aa, Vibrio campbellii
MKLPSSLAYERSIDPSDVCFFVVWPDDKKTPLTYTSRTLLGQMETASLAYDTSGQPKKSVTAEALAQGNPHQVDFCHVPYGASHVECCFSVSFSSELRKPYKCNSSSVKQTLVKLVGLYEKKIGWTELATRYLINICDGTWLWKNTRKAYCWNIELTPWPWNGENVKFEDIRNNYISRKDFENHKDWSGMTEMIKTAFSSSNGLAILEVKATVHLPTNAMVRPSQAFTEKENGSKNKSKSQNSRVFQHTVIDGERSPILGAFKTGAAIATIDDWYPEATETLRVGRFGVHREDVTCYRHPSTGKDLFSLLQKADHYIEVLSSNEAPDQEAINDMHFVMANLIKGGLFQHKGD

SEQ ID NO: 54 - protein, 353 aa, Vibrio sp.
MELPTNLAYERSIDPSDVCFLVVWPDGRKTPLTYTSRTVLGQMETAALAYEPSGRTKESATAEKLAQGNPHQVDFCHAPFGASHIECYFSVSFSSELRKPYKCNSSTVKHTLMQLIKAYEENIGWNELVSRYLVNICNGSWLWKNTKKAYCWDIELTPWPWAGGAVKFQNIRANYLERSDFENHKDWEAIAQMTKNAFSHSNGLAIFEVKATLRLPTNKQIFPSQAFTENESNNANKSKKQSKGRIFQSTTVDGERSPILGIYKTGAAIATIDDWYPDATEALRVGRFGVHKEDVTCYRHPSTQKDFFSILQQTESYIEALTSSDKPNQETINDLHFLVANIIKGGMFQHKGD

SEQ ID NO: 55 - protein, 640 aa, Vibrio vulnificus
MQTLKELIASNPDDLTTELKRAFRPLTPHIAIDSNELDALTILVNLTDKTDDQKDLLDRAKCKQKLRDEKWWASCINCVNYRQSHNPKFPDIRSEGVIRTQALGELPSFLLSSSKIPPYHWSYSHDSKYVNKSAFLTNEFCWDGEISCLGELLKDADHPLWNTLKKLGCYQKTCKAMAKQLADITHTTINVTLAPNYLTQISLPDSNTSYISLSPVASLSMQSHFHQRLQDENRHSAITRFSRTTNMGVTAMTCGGAFRMLKSSAKFSSTPHHRLNSKRSWLTSEHVQSLKQYQRLNKRLIPENSRIALRRKYKIEIQNMIRSWLAMQDHTLDSNILIQHLNHDLSYLGATKRFAYDPAMTKLFTELLKRELSSSINDGTQHTNGSYLVLPNIRVCSATALSSPVTVGIPSLTAFFGFVHAFERKLNSFNPTFRFNSFAICVHQLHVEKRGLTAEFVEKSNGTISAPATRDDWQCDIVFSLILNTNFTQRIDQSTLITSLPKRFARGSAKIAIDDLKHLNSFSTLEAAIQSLPTEVGRWFSLHAQPNNNLGDLLTAIKKDHHLMASCVGYHLLEEPKDKPNSLRGYKHAFAECIIGLINSITFSSETDANTIFWSLNNYQNYLMVQPRIINDETTYESSL

SEQ ID NO: 56 - protein, 640 aa, Vibrio anguilarum
MQTLKELIESTPDDLTTVLKRAFRPLTPHIAIDGNELDALTILVNLTDKTDDQKDLLDRAKCKQKLRDEKWWASCLNCVNYRQSHNPKFPDIRSEGIIRTEALGELPSFLLSSSKIPPYHWSYAHDSKYVNKSALLTNEFCWNGVISCLAELLKNVDHPLWKTLTKLGCYQKTRKAMAKKLASIAHITISMPLAPNYLTQISLPNSDTSYISLSPVASLSMQSYFYQGLQDEYRHASTTRFSRATNMGVTAMTCGGAFRMLKSNTKFSITPHHRLNSKRSWLTSENVQSLKQYQRLNKRLIPENARKALRRKYKIEIQNMVSVWLAMQDHTLDSIILVQHLNHDLSCLGATKRFAYNPVMTKLFTELLKRALSNSLNDSTHYSNGSFLVLPNIRVCGATALSSPVTVGIPSLTAFFGFVHAFERKLNRLNPTFRVESFAICVHQLHVEKRGLTAEFVEKGNGTISAPATRDDWQCDVVFSLILNTNFAQRIDQSTLITLLPKRFARGSAKIAIDDFKHINSFSTLEAAIQSLPIEAGRWLSLYAQPNNNLGDLLAAMKEDHQLMASCVGYHLLEEPKDKPNSLRSYKHAFAECIIGLINSITFSSETDANTIFWSLNNHQNYLVVQPRIINDETTDKSSL

SEQ ID NO: 57 - protein, 640 aa, Vibrio campbellii
MQTLKELIASNPDDLKATLKRVFRPLTPHIAIDGKELDALTILVNLTDKTDDQKDLLDRAKCKQKLRDEKWWASCLNGVNYRQSHNPKFPDIRSEGIIRTEALSELPSFLLSSSKIPPYHWSYAHDSKYVNKSAFLTNEFCWNGGISCLAELLRDVDHPLWKTLAKLGCYQKTRKAIAKQLASIAHMTISVPLAPNYLTQISLPDSNTSYISLSPVTSLSMQSHFYQGLQDEYLHTATTRYSRSTNMGVTAMTCDGAFRMLKSNTKISTTPHHRLNSKRSWLTSEHVQSLTKYQRLNKRLMPENARKALRRKCKIEVLNLVNAWLAMQDCTLDSTILVQHLNHDLSCIGATKRFAYDPAMTKLFTELLKRALSNSINNGTQHTNGSYLVLPNIRVCGATALSSPVTVGVPSLTAFFGYVHAFERKLNSIYTAFRIESFAICIHQLHVEKRGLTAEFVAKGNGTISAPATHDDWQCDAVFSLILNTNFAQRIDQKTLITALPKRLARGSAKIAIDDFKYLNSFTTIETAIQSLPIKVGRWISLHTQPNNNLGDLLAAIKEDSQLMASCVGYHLLEEPKDKPNSLRGYKHAFAESIVGLIKSITFGSTTDANTILWSLNSHKNYLVVQPRSISDETTLESSL

SEQ ID NO: 58 - protein, 230 aa, Artificial sequence
  synthetic sequence
MATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYG

SEQ ID NO: 59 - protein, 177 aa, Artificial sequence [sequence omitted]

SEQ ID NO: 60 - protein, 193 aa, Artificial sequence
  synthetic sequence
FICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 61 - protein, 286 aa, Artificial sequence
  synthetic sequence
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW